(19) United States
(12) Patent Application Publication
(10) Pub. No.: US 2012/0015899 A1
(43) Pub. Date: Jan. 19, 2012

(54) MODIFIED PLANT VIRUS PARTICLES AND USES THEREFOR

(75) Inventors: George Lomonossoff, Norwich (GB); David Evans, Norwich (GB); Elisabet de los Pinos, Brookline, MA (US); Frank Sainsbury, Quebec (CA)

(73) Assignees: Plant Bioscience, Limited, Norwich (GB); Aura Biosciences, Cambridge, MA (US)

(21) Appl. No.: 13/125,482

(22) PCT Filed: Oct. 25, 2009
(86) PCT No.: PCT/US09/05808

§ 371 Date: Sep. 12, 2011

Related U.S. Application Data
(60) Provisional application No. 61/197,400, filed on Oct. 25, 2008.

(51) Int. Cl. A61K 31/7068; C12N 1/19; C12N 1/21; A61P 35/00; C12N 5/07

(52) U.S. Cl. 514/49; 435/325; 435/252.3; 435/254.2

(57) ABSTRACT

Aspects of the invention provide modified virus-like particles that are designed for therapeutic applications. In particular, aspects of the invention provide CCMV coat proteins that are modified to generate virus-like particles, including mosaic virus-like particles, that can package and/or deliver one or more diagnostic and/or therapeutic agents. The invention also provides methods for treating subjects with one or more modified virus-like particles.

### RELATED APPLICATIONS

This application claims the benefit of the filing date of U.S. provisional patent application 61/197,400, filed Oct. 25, 2008 and entitled “Modified Plant Virus Particles and Uses Therefor”. The entire teachings of the referenced provisional patent application are expressly incorporated herein by reference.

### BACKGROUND OF THE INVENTION

The efficacy of many new classes of pharmaceuticals and biologics (e.g., peptides, proteins and DNA-based therapeutics) as well as many traditional therapeutics based on small molecules is often limited by difficulties delivering these agents in vivo. Many drugs typically cannot be effectively delivered by conventional means, such as oral ingestion, injection, or inhalation. Not only are many drugs subjected to rapid degradation or metabolism, but they often are characterized by general low bioavailability, and systemic administration often causes many undesired side-effects.

For example, although oral delivery is probably the most widely accepted form of drug delivery, it presents difficulties for a number of important classes of drugs where oral delivery mechanisms can only provide a bioavailability of a few percent, and dose limiting toxicity levels are caused by lack of selectivity. Accordingly, there is an unmet need for delivery strategies that increase drug half-life, bioavailability and targeted, sustained release of key drugs.

### SUMMARY OF THE INVENTION

Aspects of the invention relate to drug delivery methods and compositions. In particular, aspects of the invention relate to virus-like particles (VLPs) containing one or more viral coat proteins that have been modified to deliver a heterologous agent to a subject (e.g., a human subject, or an animal subject). In some embodiments, viral coat proteins are modified to improve loading and/or stable packaging of a heterologous agent. In some embodiments, viral coat proteins are modified to improve stability of the coat protein and associated heterologous agent within a subject (e.g., within the plasma of a subject). In some embodiments, viral coat proteins are modified to enhance the release of a heterologous agent at a target site (e.g., within a target cell) of a subject. In some embodiments, mosaic VLPs are used. Mosaic VLPs include two or more different viral coat proteins in a single particle. One of the coat proteins may be a wild-type protein. However, both or all of the different coat proteins may be modified. In some embodiments, one of the coat proteins is modified to contain a targeting motif such as an RGD motif. Aspects of the invention are based, at least in part, on i) the discovery that RGD motifs can destabilize VLPs, and ii) the identification of mosaic structures that can form stable VLP preparations that incorporate coat proteins modified to include an RGD motif. Mosaic VLPs can be used to deliver one or more therapeutic or diagnostic agents as described herein. In some embodiments, a mosaic may include coat proteins that are modified to include a targeting motif and coat proteins that are modified (e.g., with an N-terminal deletion and/or modification) to facilitate the loading and/or delivery of a heterologous agent.

It should be appreciated that a heterologous agent may be a diagnostic agent, a reporter molecule (e.g., gene, protein, and/or RNA), and/or a therapeutic molecule. A therapeutic molecule may be a small molecule, a polypeptide, a nucleic acid (e.g., an RNA, a DNA, or other natural or synthetic nucleic acid molecule) a gene encoding a polypeptide, any other naturally occurring or synthetic therapeutic molecule, or any combination of two or more thereof.

Aspects of the invention may be used to package and/or deliver a therapeutic agent that is an anti-cancer drug. In some embodiments, an anti-cancer drug may be 5-fluorouracil, leucovorin, capecitabine, cyclosphosphamide, docetaxel, placitaxel, or gemcitabine. In some embodiments, an anti-cancer drug may be a platin-based drug such as cisplatin, carboplatin, oxaliplatin, or satraplatin. However, other anti-cancer drugs may be used as described as the invention is not limited in this respect.

Aspects of the invention may be used to package and/or deliver a therapeutic agent that is useful to treat an infection, an inflammatory disorder, cancer, and/or any other disease or disorder.

Aspects of the invention may be used to package and/or deliver a nucleic acid that may be used to silence the expression of one or more target genes. For example, methods and compositions of the invention may be used to deliver an siRNA, an antisense RNA, or any combination thereof. It should be appreciated that RNAi and/or antisense RNA may be used to treat cancer, an infectious disease (e.g., hepatitis B or C), or any other disease or disorder as described herein.

It should be appreciated that the choice of VLP may be governed in some embodiments, at least in part, by the ability to disassemble the VLPs after their production and then to reassemble them in the presence of a heterologous agent and/or to load them with the heterologous agent after assembly such that said agent is encapsulated. A number of VLPs are potentially suitable for applications described herein, including the bacteriophages MS, Qβ, R17, fr, GA, Sp, MI, I, MXI, NL95, AP205, f2, PP7, and the plant viruses Turnip crinkle virus (TCV), Tomato bushy stunt virus (TBSV), Southern bean mosaic virus (SBMV) and members of the genus Bromovirus including Broad bean mottle virus, Brome mosaic virus, Cassia yellow blotch virus, Cowpea chlorotic mottle virus (CCMV), Melandrium yellow fleck virus, and Spring beauty latent virus. However, other VLPs also may be used as aspects of the invention are not limited in this respect.

It should be appreciated that in some embodiments VLP coat proteins may be isolated directly from an expression system without isolating and disassembling a formed VLP. Certain variant coat proteins may self-assemble less efficiently than wild-type or other variant coat proteins. However, according to aspects of the invention, variant coat proteins that are self-assembly defective may self-assemble under certain conditions and/or in the presence of one or more efficiently self-assembling variants and/or wild-type coat proteins. Accordingly, poorly self-assembling variants are nonetheless referred to as self-assembling proteins herein to indicate that they are capable of forming VLPs or mosaic VLPs under certain conditions.

In some embodiments, a mosaic VLP is prepared by mixing two or more different coat proteins under conditions that promote reassembly of a VLP. One or more of the different coat proteins may be obtained either directly from an expression system, from disassembly of a VLP (e.g., a homogeneous VLP), or from any other suitable source or combination thereof. In some embodiments, a reassembled VLP preparation may be used without any further processing. In some embodiments, a reassembled VLP preparation may be further processed (e.g., to add one or more agents, to remove non-assembled coat proteins, to remove precipitated VLP or coat protein, to sterilized the preparation, etc., or any combination thereof) to form a VLP preparation that is used, for example, in therapy (e.g., administered to a subject, e.g., a human subject).

In some embodiments, aspects of the invention relate to compositions comprising a VLP preparation, e.g., a mosaic VLP preparation. In some embodiments, provided herein are virus-like particle (VLP) preparations comprising a mosaic VLP of two or more different self-assembling CCMV coat proteins, wherein at least one of the coat proteins is modified to include a targeting peptide that comprises an integrin-binding sequence.

In some embodiments, provided herein are VLP preparations comprising a mosaic VLP of two or more different self-assembling CCMV coat proteins wherein at least one of the coat proteins has a N-terminal deletion within the first 26 amino acids and wherein the deletion is of 1 to 26 amino acids in length.

In some embodiments, provided herein are VLP preparations comprising a mosaic VLP of two or more different self-assembling CCMV coat proteins, wherein at least one of the coat proteins comprises a bacteriophage coat protein sequence.

In some embodiments, provided herein are VLP preparations comprising a mosaic VLP of two or more different self-assembling CCMV coat proteins, wherein at least one of the coat proteins comprises one or more amino acid substitutions within the first 26 N-terminal amino acids.

In some embodiments, provided herein are VLP preparations comprising a mosaic VLP of two or more different self-assembling CCMV coat proteins, wherein at least one of the coat proteins comprises a moiety selected from the group consisting of polyethylene glycol (PEG), hyaluronic acid, a natural or synthetic polymer, a histidine tag, folic acid, a second targeting peptide not comprising an integrin-binding sequence, an antibody or functional fragment thereof, and a receptor ligand molecule.

In some embodiments, provided herein are VLP preparations comprising a mosaic VLP of two or more different self-assembling CCMV coat proteins, wherein at least two of the coat proteins are modified according to any of the forgoing claims.

In some embodiments, provided herein are VLP preparations comprising a mosaic VLP of two or more different self-assembling CCMV coat proteins, wherein at least one of the coat proteins is modified using any of the techniques described herein and at least one coat protein is unmodified.

Aspects of the invention are based, at least in part, on the recognition that specific combinations of subunits aid the assembly of specific therapeutically useful mosaic VLPs. In some embodiments, mosaics are useful to promote VLP formation including one or more targeting peptides such as integrin-binding motifs. Integrin-binding motifs are useful moieties for in vivo targeting of drugs and/or delivery vehicles, such as VLPs.

In certain embodiments, VLPs are provided that comprise one or more peptides that are nine amino acids in length and that contain an RGD sequence in a cyclic conformation with two disulfide bonds that are highly selective for the av-integrins. In certain embodiments, VLPs are provided that comprise one or more peptides that are about nine amino acids in length (e.g., 5, 6, 7, 8, 9, 10, 11, 12, 13, 14, or 15 amino acids in length) and that contain an RGD sequence in a cyclic conformation with two disulfide bonds. In certain embodiments, the amino acids that preceed the RGD motif are serine and/or threonine. In certain embodiments, the amino acids that follow RGD are glycine and/or serine. In certain embodiments, the amino acids that preceed the RGD motif are serine and/or threonine and the amino acids that follow RGD are glycine and/or serine. In certain embodiments, the amino acids that are adjacent to the N-terminus of the RGD tri-peptide motif also include residues with hydrophobic or charged side chains. In certain embodiments, the RGD tri-peptide motif is flanked by cysteine residues, generating potentially cyclic disulfides, in which the RGD peptide is conformationally constrained.

In certain embodiments, targeting peptides comprise one or more αv-containing integrin-binding motifs, for example the arginine-glycine-aspartic acid (RGD) motif. In certain embodiments, RGD targeting peptides comprise the sequence motif RGDLXXL/I (SEQ ID NO: 491), wherein LXXL/I is contained within an alpha helical structure. In certain embodiments, the RGD targeting peptide is NAVPNLRGDLQVLAQKVART (SEQ ID NO: 505).

In certain embodiments, targeting peptides are organ homing peptides (e.g., that preferentially bind to a particular cell, cell-type, tissue, etc.), for example comprising the motif SRL (serine-arginine-lysine). In certain embodiments, organ homing peptides comprise the peptide sequence CLSSRLDAC (SEQ ID NO: 492). In certain embodiments, organ homing peptides comprise the motif VLR (valine-leucine-arginine). In certain embodiments, organ homing peptides comprise the peptide sequence WRCVLREGPAGGCAWFNRHRL (SEQ ID NO: 493).

Other examples of peptides are those that selectively home tumors and/or the vasculature supporting the tumor. Tumor homing peptides that contain the motif asparagines-glycine-arginine (NGR) or glycine-serine-leucine (GSL), or vasculature targeting peptides which comprise the NGR peptide.

Some integrin-binding motifs due to their structure and/or charge, such as integrin-binding motifs comprising the RGD sequence, are prone to precipitation. It was discovered by the inventors that VLPs comprising CCMV coat protein subunits comprising a RGD peptide inserted in one of the surface-exposed loops of the CCMV coat protein unexpectedly were also prone to precipitation and would not efficiently assemble into VLPs that could be used for in vivo delivery of therapeutic agents to cells and tissues expressing specific integrins. Surprisingly, it was found that careful titration of CCMV coat protein subunits with different characteristics during the VLP assembly reaction prevented precipitation of the CCMV subunit comprising the RGD peptide leading to stable mosaic VLPs. Based on this strategy, in certain embodiments, methods are provided that promote VLP assembly of subunits that would not readily assemble under normal VLP assembly conditions, for example, of subunits comprising peptides which comprise the sequence motif RGDLXXL/I (SEQ ID NO: 491), wherein LXXL/I is contained within an alpha helical structure, thereby broadening the array of therapeutically useful VLPs that can be generated. In certain embodiments, the RGD targeting peptide is NAVPNLRGDLQVLAQKVART (SEQ ID NO: 505).

In certain embodiments, mosaic VLPs are provided that are mosaic on the outer and inner surface of the VLP. Aspects of the invention are based at least in part on the recognition that by generating mosaic VLP comprising subunits with different chemical characteristics on the inner and/or the outer surface of the mosaic VLP allows tailoring of the VLP to specific therapeutic needs.

In some embodiments, mosaic VLPs are provided comprising two or more different wild-type or modified CCMV coat proteins described herein. In certain embodiments, mosaic VLPs are provided comprising two or more different coat proteins selected from the following different CCMV coat protein subunits, described herein: i) wild-type; ii) N-terminal deletion mutants (e.g., deletion of amino acids 1-5, 1-10, 1-15, 1-20, 1-25, 1-26, 1-30, 1-34, 5-10, 10-15, 15-20, 20-25, 5-25, 10-25, 15-25, 1-25, 2-25, 1-26, 2-26, 2-34, 3-26, 4-26, 5-26, 8-26 and any amino acid deletions in between); iii) N-terminal substitution mutants (e.g., substitutions that alter charged amino acids of the wild-type sequence, e.g., one or more of the 9 (e.g., 1 or more, 2 or more, 3, 4, 5, 6, 7, 8, or 9) basic residues (Arg, Lys), e.g., to net negative (Glu or Asp) residues, or any other substitutions that alter the charge based on SEQ ID NO: 1); iv) N-terminal substitution mutants e.g., comprising portions of the MS2 coat protein; v) chimeric fusion proteins comprising one or more targeting peptides in one or more of the surface exposed loops (e.g., in amino acids 52-176 of the coat protein comprising the five exterior surface-exposed loops, βB-βC (CAAAEAK (SEQ ID NO: 18), aa59-65), βC-αCD1 (ISLP (SEQ ID NO: 19), aa72-75), βD-βE (LPSVSGT (SEQ ID NO: 20), aa98-104), βF-βG (NSKDVVA (SEQ ID NO: 21), aa129-135), βH-βI (SAALTEGD (SEQ ID NO: 22), aa161-168); vi) wild-type or modified subunits comprising chemically attached targeting moieties (e.g., antibodies or antibody fragments, signaling or targeting peptides, or receptor ligand molecules); and/or vii) wild-type or modified subunits comprising chemically conjugated moieties that e.g., reduce in vivo immunogenicity of the VLP (e.g., PEG) or aid cellular uptake or themselves provide attachment points for further moieties (e.g., HA). It should be appreciated that the two or more different CCMV coat proteins may be different variants within any one of categories ii)-vii). In some embodiments, all of the different CCMV coat proteins in a VLP preparation may be different variants within any one of categories ii)-vii). In some embodiments, a mosaic VLP preparation may include 2, 3, 4, 5, 6, 7, 8, 9, 10, or more different VLP coat proteins.

In certain embodiments, modified CCMV coat proteins are provided that each can comprise one or more features, such as those described in i) and vi); i) and vii); ii) and v); ii) and vi); ii) and vii); iii) and v); iii) and vii); iii) and vii); iv) and v); iv) and vi); iv) and vii). In certain embodiments, mosaic VLP are provided comprising two or more subunits comprising one or more of the features described in i) to vii). In certain embodiments, mosaic VLP are provided comprising different ratios of the two or more different subunits comprising one or more of the features described in i) to vii).

In some embodiments, to ensure the most efficient incorporation of a heterologous molecule (e.g., drug), the pKa of the interior surface of the coat protein of a VLP can be adjusted to promote appropriate interactions between the coat protein and the heterologous molecule. In certain embodiments, the hydrophobicity of the interior surface of the coat protein of the VLP may be modified to match that of the heterologous molecule.

In some embodiments, a heterologous agent (e.g., a diagnostic agent or therapeutic agent) also is modified to be compatible with the modified VLP. For example, the heterologous agent may be modified to improve efficient packaging within a VLP, to improve stability within the VLP, and/or to improve release of the agent at the desired location (e.g., tissue or cell) within a subject.

In some embodiments, VLPs may be modified to contain one or more targeting moieties (e.g., targeting peptides) for target tissues or cell types within a subject.

In some embodiments, VLPs may be modified to improve delivery within an endosome (e.g., at relatively low pH and/or under low divalent cation concentrations).

In some embodiments, VLPs may be modified to reduce immunogenicity within a subject (e.g., within a human subject, or an animal subject).

Modifications may be changes in charge, changes in hydrophobicity, changes in salt bridges, or any combination thereof.

Modifications may be chemical modifications (e.g., pegylation or hyaluronic acid modification). In some embodiments, a VLP may be a mosaic of modified (e.g., pegylated) and non-modified coat proteins. In some embodiments, a VLP may be a mosaic of two or more differently modified coat proteins with or without other non-modified coat proteins (e.g., wild-type coat proteins). It should be appreciated, that each modified coat protein molecule may contain one or more types and/or examples of modifications described herein.

In some embodiments, a composition of the invention comprises a mosaic VLP preparation wherein a targeting peptide is integrated within one or more of the five exterior surface-exposed loops, selected from the group consisting of βB-βC (CAAAEAK (SEQ ID NO: 18), aa59-65), βC-αCD1 (ISLP (SEQ ID NO: 19), aa72-75), βD-βE (LPSVSGT (SEQ ID NO: 20), aa98-104), βF-βG (NSKDVVA (SEQ ID NO: 21), aa129-135), and βH-βI (SAALTEGD (SEQ ID NO: 22), aa161-168), on one or more of the different coat proteins in the mosaic. In some embodiments, at least two different CCMV coat proteins are present in a relative ratio of 1:1, 2:1, 3:1, 4:1, 5:1, 6:1, 7:1, 8:1, 9:1, 10:1, or at a higher ratio. In some embodiments, the coat protein that is present at a higher level in a mosaic VLP preparation is the one that forms a more stable VLP alone, and/or self-assembles alone more efficiently to form a VLP. In some embodiments, a mosaic VLP preparation comprises 2, 3, 4, 5, 6, 7, 8, 9, 10 or more different CCMV coat proteins.

In certain embodiments, methods of preparing a mosaic VLP preparation are provided, the method comprising combining at least two different CCMV coat proteins, wherein at least one coat protein is modified as described herein so that a mosaic VLP is generated. In some embodiments, at least two different CCMV coat proteins are mixed in a relative ratio of 1:1, 2:1, 3:1, 4:1, 5:1, 6:1, 7:1, 8:1, 9:1, 10:1, or at a higher ratio in a reassembly reaction to form a mosaic VLP preparation. In some embodiments, the coat protein that is provided at a higher level in the reassembly reaction is the one that forms a more stable VLP alone, and/or self-assembles alone more efficiently to form a VLP. In some embodiments, 2, 3, 4, 5, 6, 7, 8, 9, 10 or more different CCMV coat proteins are used in the reassembly reaction.

In certain embodiments, the methods further comprise combining a therapeutic molecule, a diagnostic molecule, a heterologous nucleic acid, and/or other agent with the at least two different CCMV coat proteins during reassembly. In some embodiments, a therapeutic molecule, diagnostic molecule, heterologous nucleic acid, and/or other agent is loaded into the VLP after assembly, regardless of whether the therapeutic molecule, diagnostic molecule, heterologous nucleic acid, and/or other agent was present during assembly. Accordingly, aspects of the invention relate to mosaic VLP preparations that are essentially empty shells that do not contain any additional agent or large molecule (e.g., other than water or a suitable buffer). Such preparations may be provided for subsequent loading with an agent of interest. It should be appreciated that the relative concentrations of agent and CCMV coat proteins during assembly, and/or during subsequent loading of a VLP preparation may be optimized to achieve desired levels of the agent within the VLP preparation. Accordingly, certain agents (e.g., nucleic acids, small molecules, peptides, proteins, etc.) that are inefficiently loaded can nonetheless be incorporated into VLP preparations (e.g., mosaic VLP preparations) by using sufficiently high amounts of the agent and/or optimizing the reassembly and/or loading conditions to promote loading of the agent.

In some embodiments, VLPs may be formulated for delivery to a subject (e.g., a human subject. In some embodiments, VLPs may be formulated for oral delivery. However, any form of delivery may be used as the invention is not limited in this respect. In some embodiments, VLP preparations and/or the components of the VLP preparations may be sterilized for storage and/or administration to a subject. It should be appreciated that any suitable sterilization technique may be used. However, the selection of a suitable sterilization technique may be based in part on the size of the VLP, the properties of the VLP coat proteins and/or the agent(s) contained within the VLP.

It should be appreciated that the aspects of the invention described herein may be used in conjunction with coat protein molecules each having one or more of the types or examples of modifications described herein as the invention is not limited in this respect.

Aspects of the invention relate to therapeutic methods for treating one or more conditions, including cancer, infection, and/or other diseases or conditions.

In certain embodiments, methods of treating a subject having an adverse condition are provided, the methods comprising administering to the subject a VLP preparation described herein or a pharmaceutical composition comprising a VLP preparation and optionally a non-VLP pharmaceutical composition in an amount effective to treat the condition. In certain embodiments, the adverse condition is a tumor, asthma, liver disease, heart disease, and/or Alzheimer's disease, but is not so limited. In certain embodiments, where the adverse condition is a tumor, the tumor can be a melanoma, squamous cell carcinoma, gastric, colon, non small cell lung cancer, or breast cancer, but is not so limited.

In certain embodiments, uses of a VLP preparation for preventing or treating an adverse condition are provided. Further, in certain embodiments, uses of a VLP preparation for the manufacture of a medicament for preventing or treating an adverse condition are provided.

Aspects of the invention relate to pharmaceutical compositions comprising a VLP preparation described herein optionally further comprising a non-VLP pharmaceutical compound.

Aspects of the invention are described in connection with modified VLPs. However, it should be appreciated that aspects of the invention also provide modified viral coat proteins (e.g., isolated and/or purified coat proteins) regardless of whether they are assembled to form VLPs, nucleic acids (e.g., isolated and/or purified nucleic acids) that encode one or more coat proteins and/or heterologous agents described herein, related vectors and/or host-cells, VLPs (e.g., isolated and/or purified VLPs) with or without packaged heterologous agent, or any combination thereof.

### BRIEF DESCRIPTION OF THE DRAWINGS

FIG. 1 is a non-limiting illustration of the synthetic CCMV coat protein gene structure showing (A) specific sites and functions, and (B) wild-type and modified nucleotide sequences (mutations that generate amino acid substitutions at the following residues in the corresponding amino acid sequence: R26H, P75G, L131R).

FIG. 2 is a non-limiting illustration of the CCMV coat protein structure showing surface exposed loops (loops that were tested for targeting peptide insertions are in bold), core structure, C-terminus and non-resolved (highly mobile) N-terminal RNA binding domain.

FIG. 3 is a non-limiting illustration of the synthetic CCMV coat protein gene cloned into pPICZ vector (A) pPICZ-CCMV, and (B) pPICZ-CCMV/N1.

FIG. 4 is a non-limiting illustration of (A) Coomassie stain of purified CCMV coat protein (wt: wild-type, N1: N-terminal deletion, amino acids 8-26) on 12% PAGE, and (B) Coomassie stain (left) of purified CCMV coat protein (wt, N1) and ethidium bromide stain on 1% agarose gel (right).

FIG. 5 is a non-limiting illustration of electron microscopic images of reassembled VLP of wild-type (left) and N1 (right).

FIG. 6 is a non-limiting illustration of the synthetic CCMV coat protein gene cloned into pPICZ vector (A) pPICZ-CCMV-RGD-bCaCD1, (B) pPICZ-CCMV-RGD-bFbG, (C) pPICZ-CCMV/N1-RGD-bCaCD1, and (D) pPICZ-CCMV/N1-RGD-bFbG.

FIG. 7 is a non-limiting illustration of a western blot showing reassembled N1 and RGD-bCaCD1 VLPs when mixed together in different ratios.

FIG. 8 is a non-limiting illustration of various cloning constructs and sequences.

### DETAILED DESCRIPTION OF THE INVENTION

Described herein are delivery agents, such as plant virus particles, for the delivery of agents such as diagnostic or therapeutic agents (e.g., anti-cancer agents). By “delivery agent,” “delivery vehicle” or “protein carrier” herein is meant a proteinaceous shell that self-assembles to form a structure with an interior cavity which is either naturally accessible to a solvent or can be made to be so by altering for example the solvent concentration, pH, or equilibria ratios, and that may contain one or more agents as discussed herein.

In some embodiments, a delivery vehicle is based on a modified Bromovirus particle such as a Cowpea chlorotic mottle virus (CCMV) particle. CCMV (Speir, J. A., et al., 1995, Structure 3:63-78) is a member of the bromovirus group of the Bromoviridae (Ahlquist, P., 1992, Curr. Opin. Gen. and Dev. 2:71-76; Dasgupta, R., and P. Kaesberg, 1982, Nucleic Acid Res. 5:987-998; and Lane, L. C., 1981, The Bromoviruses. In E. Kurstak (ed.), “Handbook of plant virus infection and comparative diagnosis,” Elsevier/North-Holland, Amsterdam). Bromoviruses are 25-28 nm icosahedral viruses with a four component (+) sense single stranded RNA genome. Purified CCMV RNA and CCMV coat protein self-assemble in vitro to produce infectious virions (Bancroft, J. B., et al., 1969, Virology 38:324-335; Bancroft, J. B., and E. Hiebert, 1967, Virology 32:354-356; Bancroft, J. B., et al., 1968, Virology 36:146-149; Hiebert, E., and J. B. Bancroft, 1969, Virology 39:296-311; and Hiebert, E., et al., 1968, Virology 34:492-508). CCMV undergoes a reversible pH-dependent structural transition between a closed and open form resulting in the opening of 60 pores of approximately 2 nm in diameter allowing access between the interior and exterior environments.

In some embodiments, empty or hollow virus-like particles (VLPs) are obtained from CCMV or other plant virus or other non-plant virus (e.g., Hepatitis B core antigen, Human Papiloma virus, human immunodeficiency virus, human influenza virus, etc.). An “empty” or “hollow” VLP comprises a membrane-enclosed vesicle, which can provide a luminal space, which is filled with a substance, which can be any gaseous, liquid, semi-solid or solid substance. A VLP may generally be spherical, but may have other shapes. The “membrane” can consist of any material, for example, lipids, proteins, polysaccharides, other carbohydrates, synthetic or natural polymers. In certain embodiments, the membrane comprises wild-type or modified CCMV coat protein. VLPs described herein may be used in some embodiments to deliver therapeutic agents to a subject in need of such a therapeutic intervention.

Aspects of the invention are based, at least in part, on the recognition that the functionality of a VLP (therapeutic agent loading capacity/capability, in vivo immunogenicity, cell or tissue specificity/targeting, stability, etc.) can be fine-tuned by assembly of different mosaic VLP comprising subunits with one or more different features in different ratios.

In some embodiments, mosaic VLPs are provided comprising two or more different CCMV coat proteins described herein (e.g., wild-type and/or modified). In certain embodiments, mosaic VLPs are provided comprising two or more different CCMV coat protein subunits, independently selected from the following: i) wild-type; ii) N-terminal deletion mutants (e.g., deletion of amino acids 1-5,1-10, 1-15, 1-20, 1-25, 1-26, 1-30, 1-34, 5-10, 10-15, 15-20, 20-25, 5-25, 10-25, 15-25, 2-25, 2-26, 2-34, 3-26, 4-26, 5-26, 8-26 and any amino acid deletions in between); iii) N-terminal substitution mutants (e.g., substitutions that alter charged amino acids of the wild-type sequence, e.g., one or more of the 9 (e.g., 1 or more, 2 or more, 3, 4, 5, 6, 7, 8, or 9) basic residues (Arg, Lys), e.g., to net negative (Glu or Asp) residues, or any other substitutions that alter the charge based on SEQ ID NO:1); iv) N-terminal substitution mutants e.g., comprising portions of the MS2 coat protein; v) chimeric fusion proteins comprising one or more targeting peptides in one or more of the surface exposed loops (e.g., in amino acids 52-176 of the coat protein comprising the five exterior surface-exposed loops, βB-βC (CAAAEAK (SEQ ID NO: 18), aa59-65), βC-αCD1 (ISLP (SEQ ID NO: 19), aa72-75), βD-βE (LPSVSGT (SEQ ID NO: 20), aa98-104), βF-βG (NSKDVVA (SEQ ID NO: 21), aa129-135), βH-βI (SAALTEGD (SEQ ID NO: 22), aa161-168); vi) wild-type or modified subunits comprising chemically attached targeting moieties (e.g., antibodies or antibody fragments, signaling or targeting peptides, or receptor ligand molecules); and/or vii) wild-type or modified subunits comprising chemically conjugated moieties that e.g., reduce in vivo immunogenicity of the VLP (e.g., PEG) or aid cellular uptake or themselves provide attachment points for further moieties (e.g., HA). It should be appreciated that the two or more different CCMV coat proteins may be different variants within any one of categories ii)-vii). In some embodiments, all of the different CCMV coat proteins in a VLP preparation may be different variants within any one of categories ii)-vii). In some embodiments, a mosaic VLP preparation may include 2, 3, 4, 5, 6, 7, 8, 9, 10, or more different VLP coat proteins.

In certain embodiments, modified CCMV coat proteins are provided that each can comprise one or more features, such as those described in i) and vi); i) and vii); ii) and v); ii) and vi); ii) and vii); iii) and v); iii) and vii); iii) and vii); iv) and v); iv) and vi); iv) and vii). In certain embodiments, mosaic VLPs are provided comprising two or more subunits comprising one or more of the features described in i) to vii). In certain embodiments, mosaic VLPs are provided comprising different ratios of the two or more different subunits comprising one or more of the features described in i) to vii).

It should be appreciated that certain variant coat proteins may self-assemble at different rates and/or to different extents. In some embodiments, a variant coat protein may self-assemble less efficiently than a wild-type protein. In some embodiments, assembly of a variant coat protein may be increased by altering the assembly conditions and/or by assembling the variant in the presence of a different coat protein (e.g., a wild-type coat protein or a coat-protein variant that assembles more efficiently) to form a mosaic VLP. As used herein, a self-assembling coat protein refers to both wild-type and variant coat proteins, including variant coat proteins that precipitate when alone and/or that do not assemble efficiently relative to wild-type coat proteins when alone, but for which assembly can be restored, for example, by reassembling the variant in the presence of a different variant and/or a wild-type coat protein.

In certain embodiments, VLPs are provided comprising one subunit comprising an N-terminal deletion of the CCMV coat protein (e.g., amino acids 8-26, 1-25 or 1-26) and one subunit comprising a RGD targeting peptide integrated into a surface-exposed loop of the CCMV coat protein. In a particular embodiment, VLPs are provided comprising one subunit comprising an N-terminal deletion of the N-terminal amino acids 8-26 of the CCMV coat protein and one subunit comprising the RGD-4C targeting peptide integrated into the βCαCD surface-exposed loop of the CCMV coat protein.

Aspects of the invention are based, at least in part, on the recognition that specific combinations of subunits aid the assembly of specific therapeutically useful mosaic VLPs. Peptides comprising integrin-binding motifs, such as those comprising a RGD motif, are useful moieties for in vivo targeting of drugs and/or delivery vehicles, such as VLPs. However, it was found that peptides comprising the RGD motif are prone to precipitation, possibly due to their specific structure and/or charge. It was discovered by the inventors that VLPs comprising CCMV coat protein subunits comprising a RGD peptide inserted in one of the surface-exposed loops of the CCMV coat protein was also prone to precipitation and would not efficiently assemble into VLPs that could be used for in vivo delivery of therapeutic agents to cells and tissues expressing specific integrins. This was surprising, because the art suggested that CCMV coat proteins comprising targeting peptides inserted into surface exposed loops (such as peptide 11) could be expressed and assembled into VLPs near wild-type levels (e.g., WO 2008/048288) and suggested that other (targeting) peptides should likewise be expressed. According to the invention, several possible routes to solve the problem of precipitation can be contemplated, such as making the insertions of the targeting peptide in different positions of the five exterior surface-exposed loops, βB-βC (CAAAEAK (SEQ ID NO: 18), aa59-65), βC-αCD1 (ISLP (SEQ ID NO: 19), aa72-75), βD-βE (LPSVSGT (SEQ ID NO: 20), aa98-104), βF-βG (NSKDVVA (SEQ ID NO: 21), aa129-135), βH-βI (SAALTEGD (SEQ ID NO: 22), aa161-168); modifying (genetically, chemically) the charge, influencing the (secondary) structure or varying the length of the targeting peptide; altering the buffer conditions (e.g., salt, pH, ions) and/or protein concentration (dilute/concentrate) of the added CCMV subunits for the VLP assembly reaction. Surprisingly, it was found that careful titration of CCMV coat protein subunits with different characteristics during the VLP assembly reaction prevented precipitation of the CCMV subunit comprising the RGD peptide leading to stable mosaic VLPs.

Integrins are alpha-beta heterodimers expressed on a wide variety of cells. The ligands for several of the integrins are extracellular matrix proteins such as fibronectin, vitronectin, collagens, and laminin. Many of the integrins recognize the sequence RGD in fibronectin and a number of other adhesive proteins (Piershbacher and Ruoshlati, 1984, Ruoshlati and Piershbacher, 1987). This tri-peptide sequence is recognized at least by the integrins α5β6, (Pytela et al, 1985), αvβ1 (Vogel et al, 1990), αIIβvβ3 (Plow et al, 1985), αvβ3 (Pytela et al 1985), αvβ5 (Cheresh et al, 1989), avb6 (Busk et al, 1992). In certain embodiments, VLPs are provided that comprise one or more peptides that are nine amino acids in length and that contain an RGD sequence in a cyclic conformation with two disulfide bonds and that are highly selective for the αv-integrins. In certain embodiments, VLPs are provided that comprise one or more peptides that are about nine amino acids in length (e.g., 5, 6, 7, 8, 9, 10, 11, 12, 13, 14, or 15 amino acids in length) and that contain an RGD sequence in a cyclic conformation with two disulfide bonds. In certain embodiments, the amino acids that preceed the RGD motif are serine and/or threonine. In certain embodiments, the amino acids that follow RGD are glycine and/or serine. In certain embodiments, the amino acids that preceed the RGD motif are serine and/or threonine and the amino acids that follow RGD are glycine and/or serine. In certain embodiments, the amino acids that are adjacent to the N-terminus of the RGD tri-peptide motif also include residues with hydrophobic or charged side chains. In certain embodiments, the RGD tri-peptide motif is flanked by cysteine residues, generating potentially cyclic disulfides, in which the RGD peptide is conformationally constrained. In certain embodiments, the RGD targeting peptide is NAVPNLRGDLQVLAQKVART (SEQ ID NO: 505).

It should be appreciated that the outer surface of a VLP is defined by certain portions of the coat proteins that are exposed on the outer surface of the assembled VLP (for example, the surface exposed loops of the CCMV coat protein). Similarly, the inner surface of a VLP is defined by certain portions of the coat proteins that are exposed on the inner surface of the assembled VLP (e.g., the N-terminal region of the CCMV coat protein). The inner and outer portions can be determined from the structure (e.g., the crystal structure) of an assembled VLP, or using any other suitable technique. In certain embodiments, mosaic VLPs are provided that are mosaic on the outer and inner surface of the VLP. In certain embodiments, the inner surface of the mosaic VLP comprises a mixture of CCMV coat protein subunits that have either N-terminal alterations, deletions, substitutions or are wild-type, in various ratios. In certain embodiments, the outer surface of the mosaic VLP comprises a mixture of CCMV coat protein subunits that have either one or more different targeting moieties (e.g., chemically linked or genetically inserted), moieties that reduce immunogenicity or are wild-type, in various ratios. In certain embodiments, the inner and the outer surface of the mosaic VLP comprises a mixture of CCMV coat protein subunits that have specific features, thereby allowing tailoring of the VLP to specific therapeutic needs and/or enabling VLP assembly of subunits that would not readily assemble under normal VLP assembly conditions, thereby broadening the array of therapeutically useful VLPs that can be generated.

In certain embodiments, methods of producing mosaic VLPs that are mosaic on the outer and/or inner surface of the VLP are provided. These methods allow the assembly of CCMV coat protein subunits into VLP that would not readily assemble under normal VLP assembly conditions, such as subunits comprising peptides that are large (e.g., 10, 15, 20, 25, 30, 40, or more amino acids) in that they may disrupt the overall CCMV coat protein structure due to size; strongly charged (basic, acidic), particularly hydrophobic or amphipathic; peptides that may disrupt the overall CCMV coat protein structure due to a particular secondary structure of the peptide. Such peptides include integrin-binding peptides, such as RGD peptides. In certain embodiments, the RGD targeting peptide is NAVPNLRGDLQVLAQKVART (SEQ ID NO: 505). Careful mixing of CCMV coat protein subunits comprising such peptides with CCMV coat protein that do not express such peptides was found to efficiently produce VLPs with the desired targeting ability in various ratios. In certain embodiments, the ratio of targeting peptide expressing subunits and non-peptide expressing subunits is 50%:50%. In other embodiments, the ratio is 10%:90%, 20%:80%, 30%:70%, 40%:60%. It should be appreciated that in some embodiments these ratios may be used as input ratios in a reassembly reaction. However, in some embodiments these ratios may be the desired output ratios from an assembly reaction and slightly different input ratios may be required in order to generate these output ratios as described herein. In any of the assembly or reassembly reactions described herein in the context of VLP or mosaic VLP preparation, the relative ratios of different coat protein preparations may be evaluated using any suitable technique for detecting and/or measuring protein concentrations or amounts. It should be appreciated that in some embodiments the ratios described herein may be approximate and similar or intermediate or higher or lower ratios also may be used.

The VLPs, in some embodiments, may be used to deliver anti-cancer agents, such as for example XELODA/Capecitabine, GEMZAR/Gemcitabine, 5-fluoro-uracil, TAXOTERE/Docataxel, CAMPTO/Irinotecan, TAXOL/Paclitaxel, and/or cisplatinum compounds in vivo to a subject having cancer.

In some embodiments, the VLPs described herein may be loaded in vitro with a therapeutic, diagnostic, or other agent by inducing the reversible pH-dependent structural transition between a closed and open form of the VLP and allowing agents to enter the VLP. Upon changing the conditions the VLP reverts back to the closed form thereby entrapping the therapeutic agent. In some embodiments, the VLPs described herein may be loaded in vitro with a therapeutic, diagnostic, or other agent by mixing the agent with one or more different CCMV coat proteins during reassembly.

In some embodiments, the VLP may consist entirely of CCMV coat proteins. In other embodiments, the VLP may further comprise one or more additional viral or heterologous proteins or protein fragments. In certain embodiments, such heterologous proteins or protein fragments may be derived from mammals, such as e.g., humans. In some embodiments, the heterologous proteins or protein fragments may comprise targeting peptides, providing means to specifically target the VLP in vivo to specific tissues or cells expressing antigens, such as tumor-associated antigens, tumor-specific antigens, tissue-specific antigens, or cell type-specific antigens.

In some embodiments, the heterologous proteins or protein fragments may comprise one or more targeting peptides, providing means to specifically target the VLP in vivo to specific tissues or cells expressing cell surface receptors.

In some embodiments, targeting peptides may selectively target the vasculature supporting the tumor. In certain embodiments, such peptides comprise the NGR amino acid sequence. Other tumor homing peptides may contain the motif glycine-serine-leucine (GSL). Other tumor homing peptides such as the peptides CGRECPRLCQSSC (SEQ ID NO: 494) and CNGRCVSGCAGRC (SEQ ID NO: 495) have been identified based on their ability to home to a breast carcinoma. The peptide CLSGSLSC (SEQ ID NO: 497) has been identified based on its ability to home to a melanoma. Such tumor homing peptides were identified using in vivo panning (see U.S. Pat. No. 5,622,699, issued Apr. 22, 1997; Pasqualini and Ruoslahti, Nature 380:364-366 (1996), each of which is incorporated herein by reference). Additional tumor homing peptides are known in the art and/or can be identified by in vivo panning.

In some embodiments, the targeting peptide identifies the lymphatic vasculature of a tumor comprising the amino acid sequence GNKRTRG (SEQ ID NO: 498). In some embodiments, the targeting peptide identifies a specific organ. In some embodiments, the targeting peptide is a brain homing peptide that comprise the motif SRL (serine-arginine-lysine), such as the peptide CLSSRLDAC (SEQ ID NO: 492) or the homing peptide comprises the motif VLR, such as the peptide WRCVLREGPAGGCAWFNRHRL (SEQ ID NO: 493). In some embodiments, the targeting peptide is a kidney homing peptide, such as CLPVASC (SEQ ID NO: 496) or CGAREMC (SEQ ID NO: 499). In some embodiments the targeting peptide is a heart homing peptide that contains the amino acid sequence GGGVFWQ (SEQ ID NO: 500), HGRVRPH (SEQ ID NO: 501), VVLVTSS (SEQ ID NO: 502), CLHRGNSC (SEQ ID NO: 503), or CRSWNKADNRSC (SEQ ID NO: 504).

In some embodiments, the peptides target a cell surface receptor which may be selected from the group consisting of insulin receptor (insulin), insulin-like growth factor receptor (including both IGF-1 and IGF-2), growth hormone receptor, glucose transporters (particularly GLUT 4 receptor), transferrin receptor (transferrin), epidermal growth factor receptor (EGF), low density lipoprotein receptor, high density lipoprotein receptor, leptin receptor, estrogen receptor (estrogen); interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor (VEGF), PDGF receptor (PDGF), transforming growth factor receptor (including TGF- and TGF-), EPO receptor (EPO), TPO receptor (TPO), ciliary neurotrophic factor receptor, prolactin receptor, and T-cell receptors.

In some embodiments, the heterologous proteins or protein fragments may comprise one or more targeting peptides, providing means to specifically target the VLP in vivo to specific tissues or cells expressing integrins, such as αvβ3, αvβ5, and αvβ6 integrins, wherein the targeting peptide comprises an RGD motif. In certain embodiments, the RGD targeting peptide is NAVPNLRGDLQVLAQKVART (SEQ ID NO: 505).

In some embodiments, the heterologous proteins or protein fragments may comprise one or more targeting peptides derived from viruses other than CCMV, providing means to specifically target the VLP in vivo to specific tissues or cells. In certain embodiments targeting peptides derived from virus that have been identified to be responsible for viral-mediated cell entry are provided. For example peptides derived from the Hepatitis B surface antigen which identifies hepatocytes may be used for the treatment of liver disease and hepatocellular carcinoma. Peptides derived from the Human Papilloma Virus which identifies cervical epithelial cells may be used for the treatment of cervical cancer and cervical dysplasia. Peptides derived from the Epstein Barr Virus which identifies lymphocytes may be used for the treatment of lymphoma.

In certain embodiments, the human complement receptor 2 (CR2) binding domain of glycoprotein gp350/220 of the Epstein-Barr virus is a virus-derived targeting peptide that may be used for targeting as described herein.

In certain embodiments, the carboxyl terminus of the HPV L1 protein is a virus-derived targeting peptide that may be used for targeting as described herein.

In certain embodiments, the pre-S2 region of HBV is a virus-derived targeting peptide that may be used for targeting as described herein.

In certain embodiments, the HCV envelope glycoproteins (HCVpp) E1 and E2, specifically amino acids 412-447 within E2 are used for targeting as described herein. In some embodiments, peptides are used to enhance oral delivery. In certain embodiments, the target tissue is follicle associated epithelium (FAE) overlying Peyer's patches which contains M-cells that have an increased capacity for uptake of particulate antigens. In certain embodiments, an integrin-adherent peptide motif, RGD, can be utilized to achieve selective and improved transport of VLPs into human Peyer's patches to improve oral delivery. In certain embodiments, the RGD targeting peptide is NAVPNLRGDLQVLAQKVART (SEQ ID NO: 505).

In certain embodiments, the targeting moiety directs the VLP to a tumor, a site of inflammation, a site of wound healing, a site of soft tissue damage, a site of bone or cartilage damage, a site of immune cell regeneration, across the blood-brain barrier, or a site of fat cell deposition.

It should be appreciated that any targeting peptide or motif described herein (or other peptide described herein) may be inserted into one or more surface exposed loops of a CCMV coat protein (e.g., in one or more copies of a CCMV coat protein in a mosaic VLP preparation). In some embodiments, a peptide may be inserted into one or more of the following five exterior surface-exposed loops, selected from the group consisting of βB-βC (CAAAEAK (SEQ ID NO: 18), aa59-65), βC-αCD1 (ISLP (SEQ ID NO: 19), aa72-75), βD-βE (LPSVSGT (SEQ ID NO: 20), aa98-104), βF-βG (NSKDVVA (SEQ ID NO: 21), aa129-135), and βH-βI (SAALTEGD (SEQ ID NO: 22), aa161-168). In some embodiments, two or more different peptides may be present in a single coat protein variant (e.g., at different positions in a single surface exposed loops or in different surface exposed loops). It should be appreciated that each peptide may be inserted at the N-terminal end, the C-terminal end, or in between, of each surface exposed loop that is so modified. In some embodiments, a stable (e.g., the most stable or one of the most stable) insertion site variants is selected, e.g., using standard techniques to evaluate the stability of the resulting VLP particle or a mosaic VLP particle containing the modified coat protein.

In a natural CCMV particle, the 180 copies of the coat protein encapsidate the viral RNAs (RNA-1, RNA-2 or RNA-3+RNA-4). However, VLPs may be produced without these RNAs. In some embodiments, VLPs with a wild type N-terminus can encapsidate heterologous RNAs from a host.

In some embodiments of the invention, the VLP may be wild-type, e.g., it may be derived directly form CCMV. However, it should be appreciated that aspects of the invention relate to a modified or altered VLP. By “modified” or “altered” herein is meant a VLP that has been genetically altered or modified by physical, chemical or biochemical means as described herein.

In some embodiments, the VLP provided is modified in that certain amino acids are genetically altered (e.g., by insertion of a point mutation via site-directed mutagenesis) providing a VLP with altered assembly, stability or disassembly characteristics, or altered loading and/or delivery capacity with regard to therapeutic agents, altered bioavailability, or other altered characteristics.

In some embodiments, the 3.2 Å resolution structure of CCMV which is publicly available may be used to predict the role of individual amino acids in controlling virion assembly, stability, and/or disassembly (Speir, J. A., et al., 1995, Structure 3:63-78). The virion is made up of 180 copies of the coat protein subunit arranged with a T=3 quasi-symmetry and organized in 20 hexamer and 12 pentameric capsomers to give particles with an external diameter of 28.6 nm. This architecture is shared by many icosahedral viruses. The individual subunits consist of 190 amino acids and have a molecular weight of 19.8 kDa. Each subunit contains several distinct domains.

In certain embodiments, the CCMV coat protein subunit features N- and C-terminal extensions that extend away from the central, eight-stranded, antiparallel β-barrel core. Each coat protein consists of a canonical β-barrel fold (formed by amino acids 52-176) from which long N-terminal (residues 1-51; 1-26 are not ordered in the crystal structure) and C-terminal arms (residues 176-190) extend in opposite directions. These N- and C-terminal extensions provide interaction surfaces between subunits.

FIG. 2 shows a non-limiting representation of a CCMV coat protein. The natural RNA-binding domain is indicated. In some embodiments, mutations of basic residues prevents encapsidation of heterologous RNA. However, in some embodiments, modifications in this region may be used to promote encapsidation of one or more different types of RNA or other agents as described herein.

The following paragraphs relate to CCMV sequences and structures. However, it should be appreciated that similar modifications may be made in other virus derived particles described herein.

Amino acids 1-26: The first 25 amino acids are found lining the interior surface of the virion and are not visible in the crystallographic structure of the virus (Rao, A. L. and G. L. Grantham, 1996, Virology 226:294-305; and, Zhao, X., et al., 1995, Virology, 207:486-494). These 25 amino acids are thought to be highly mobile and to be important for efficient viral RNA packaging. Nine of the first 25 amino acids are basic, positively charged residues (Arg, pKₐ 12.48; Lys pKₐ 10.53) and are thought to neutralize the negatively charged RNA.

Amino acids 27-51: Amino acids 27-51 form an N-terminal extension to the main β-barreldomain. Together with the C-terminal extension, these form a network which ties the subunits together in the assembled particle.

Amino acids 52-176: The orientation of the coat protein β-barrel fold, which contains 8 strands of anti-parallel β-sheet, is nearly parallel to the five-fold and quasi six-fold axes. This orientation results in five exterior loops, βB-βC, βD-βE, βF-βG, βC-αCD1, βH-βI, being exposed on the surface of the virus particle.

Amino acids 177-190: These amino acids interact both with the RNA and the 13-barrel domain and are important for stabilizing the quaternary structure of the virus.

Surrounding each of the 60 quasi three-fold axes located on the interface between hexamer and pentamer capsomers are Ca²⁺ binding sites. There are 180 Ca²⁺ binding sites per virion. Each Ca²⁺ binding site consists of five residues (Glu81, Gln85, Glu148 from one subunit; Gln 149 and Asp 153 from an adjacent subunit) in a position to coordinate Ca²⁺ binding.

In some embodiments, VLPs are provided comprising coat protein that has amino acids 1-26 deleted or modified. Amino acids 1-26 of the coat protein (MSTVGTGKLT RAQRRAAARKNKRNTR (SEQ ID NO: 1), RNA-binding domain, underlined; positively charged residues in italics), are not required for empty virion or particle assembly (devoid of viral RNA) and can be deleted without abolishing the ability of the expressed coat protein to form VLPs (Douglas et al., 2002; Adv. Mater. 14: 415-418). Deletion of amino acids 1-26 of the coat protein may be partial or complete, e.g., the deletion may comprise 1, 2, 3, 4, 5, 6, 7, 8, 9, 10, 11, 12, 13, 14, 15, 16, 17, 18, 19, 20, 21, 22, 23, 24, 25, or 26 amino acids. The deletion may be N-terminal, or may comprise any region and any number of amino acids within amino acids 1-26 of the coat protein (e.g., amino acids 8-26 or 1-26). Amino acids may also be modified. Modification of amino acids includes amino acid substitutions, which may be conservative or non-conservative substitutions. For example, one or more amino acids of amino acids 1-26 of the coat protein may be modified to change the electrostatic nature of the amino acids. The N-terminus contains nine amino acids residues with positively charged side chains (Lys8, Arg11, Arg14, Arg15, Arg19, Lys20, Lys22, Arg23, Arg26; Arg, pKₐ 12.4; Lys pKₐ 10.5) that can be modified, individually or in groups, so that the N-terminus changes its net charge, for example either with non-charged amino acid residue side chains, or negatively charged amino acid residue side chains (Asp, pKₐ 3.7; Glu pKₐ 4.3) at neutral pH. Other amino acid residue side chains that are sensitive to pH changes in the physiological range are for example His, pKₐ 6.1 and Cys, pKₐ 8.00. At pH=pKₐ 50% of molecules will be deprotonated in solution. Other amino acid residue side chains that carry no net charge and provide increased hydrophobicity are for example Phe, Tyr, or Trp.

In certain embodiments, a number of residues outside the N-terminus that contribute to an overall positively charged capsid interior may be modified provided that this modification does not affect particle assembly. In these embodiments, Lys42, Lys45, and Arg179, which are indicated on surface rendered representations of the capsid as exposed positive charge and may be involved in subunit interactions (Speir et al., 1995; Structure 3: 63-78) may be altered to carry less positive charge, no charge, more negative charge, or to carry hydrophobic residues, provided that thes modifications do not affect particle assembly.

In some embodiments, Lys42, Lys45, and Arg179 are not altered but kept constant.

In some embodiments one or more amino acids of amino acids 1-26 of the coat protein may be modified or deleted to increase the solubility of a anti-cancer drug.

In some embodiments one or more amino acids of amino acids 1-26 of the coat protein may be modified or deleted to prolong the half-life of the anti-cancer drug.

In some embodiments one or more amino acids of amino acids 1-26 of the coat protein may be modified or deleted to increase stability of the anti-cancer drug in solution.

In some embodiments one or more amino acids of amino acids 1-26 of the coat protein may be modified or deleted to avoid the need for chemical modification of the anti-cancer drug so that the drug is liberated in the cell in its active form.

In some embodiments one or more amino acids of amino acids 1-26 of the coat protein may be modified by reducing the positive charge to reduce the incorporation of heterologous RNAs during capsid assembly, which may eliminate possible competition between the heterologous RNA and the anti-cancer drug to be encapsidated.

In some embodiments, deletions or modifications may be made to provide an empty particle that then can be disassembled and reassembled in the presence of an agent of interest.

In some embodiments, one or more amino acids of N-terminal amino acids 1-26: M S T V G T G X₁ L T X₂A Q X₃ X₄ A A A X₅ X₆N X₈N T X₉ (SEQ ID NO: 2) are modified, particularly at X₁-X₉ with one or more of the following amino acid residues: Asp, Glu, His, Cys, Phe, Tyr, or Trp. In these embodiments, any particular number and combination of amino acid residues at positions X₁-X₉ may be modified with any particular combination of amino acid residues Asp, Glu, His, Cys, Phe, Tyr, or Trp, or any residue may be modified to any particular one residue of Asp, Glu, His, Cys, Phe, Tyr, or Trp.

In some embodiments, one or more of positions X₁-X₉ may be modified to any known amino acid, natural and non-natural. In some embodiments, one or more of positions X₁-X₇ may be modified to any known amino acid, natural and non-natural.

In some embodiments, positions other than X₁-X₉ in MSTVGTGX₁LTX₂AQ X₃X₄AAAX₅X₆NX₇X₈NTX₉ (SEQ ID NO: 2) may be modified to any known amino acid, natural and non-natural.

In some embodiments, positions other than X₁-X₉ in MSTVGTGX₁LTX₂AQ X₃X₄AAAX₅X₆NX₇X₈NTX₉ (SEQ ID NO: 2) are not modified and are kept constant.

For example, a positively charged N-terminus may comprise the wild-type sequence: MSTVGTGKLTRAQRRAAARKNKRNTR (SEQ ID NO: 3). A positively charged N-terminus may also comprise, for example: MSTVGTGKLTKAQKKAAAKKNKKNTK (SEQ ID NO: 4) or MSTVGTGRLTRAQRRAAARRNRRNTR (SEQ ID NO: 5).

A weakly positively charged N-terminus may comprise, for example: MSTVGTGKLTAAQAGAAAAGNAANTG (SEQ ID NO: 6), wherein, for example, all but one amino acid is positively charged and the other positive residues are, for example, modified to alanines and/or glycines, or an intermediately charged N-terminus: MSTVGTGALTRAQRGAAAVKNKLNTI (SEQ ID NO: 7), wherein, for example, four amino acids are positively charged and the other positive residues are, for example, modified to alanines, and/or glycines, and/or leucines, and/or isoleucines, and/or valines.

For example, a negatively charged N-terminus may comprise: MSTVGTGDLTDAQDDAAADDNDDNTD (SEQ ID NO: 8), or MSTVGTGELTEAQEEAAAEENEENTE (SEQ ID NO: 9).

A weakly negatively charged N-terminus may comprise, for example: MSTVGTGDLTEAQAGAAALINVANTG (SEQ ID NO: 10), wherein, for example, two amino acids are negatively charged and the other positive residues are, for example, are modified to alanines, and/or glycines, and/or leucines, and/or isoleucines, and/or valines.

Mixed charges may also be possible, for example: MSTVGTGKLTRAQEDAAARKNDENTA (SEQ ID NO: 11).

In some embodiments, the one or more positively charged wild-type amino acids may be changed to be more hydrophobic, for example: MSTVGTGYLTWAQFFAAAYYNIINTW (SEQ ID NO: 12), or MSTVGTGWLTRAQYRAAAFKNKWNTR (SEQ ID NO:13), or MSTVGTGKLTRAQEDAAAWKNFYNTR (SEQ ID NO: 14).

In some embodiments, introduction of His, Cys residues may be desired, for example: MSTVGTGHLTCAQRRAAACKNKRNTH (SEQ ID NO: 15), or MSTVGTGELTDAQHEAAADCNHHNTD (SEQ ID NO:16), or MSTVGTGCLTHAQAVAAARKNIGNTW (SEQ ID NO: 17), wherein, for example, the other positive residues are modified to alanines, and/or glycines, and/or leucines, and/or isoleucines, and/or valines, and/or tyrosines, and/or phenylalanines, and/or tryptophanes.

In some embodiments, one or more (e.g., all) of positions X₁-X₇ or X₁-X₉ may be maintained as in the wild type sequence and positions 8-25 or 8-26 or 10-25 or 10-26 may be deleted. In some embodiments, one or more (e.g., all) of positions X₁-X₇ or X₁-X₉ may be altered and positions 8-25 or 8-26 or 10-25 or 10-26 may be deleted.

In some embodiments, the net charge of the N-terminus is altered to strengthen the electrostatic interaction between the therapeutic agent and the interior surface of the VLP to enhance retention of the agent during loading. For example, additional residues in amino acids 1-26 of the N-terminus of the coat protein may be modified to carry positively charged side chains, e.g., arginine or lysine, at neutral pH, to enhance retention of negatively charged therapeutic agents, such as phosphorylated drug or pro-drug molecules.

In some embodiments, the net charge of the N-terminus is altered to weaken the electrostatic interaction between the therapeutic agent and the interior surface of the VLP to enhance delivery of the agent in the target cell. For example, one or more of amino acids 1-26 of the N-terminus of the coat protein may be modified to carry negatively charged side chains, e.g., aspatic acid or glutamic acid, to weaken retention of negatively charged therapeutic agents

It is understood that the opposite of the aforementioned situations can also be achieved. That is that an increased positive charge of the N-terminus can be used to weaken the interaction with a positively charged therapeutic agent and that an increased negative charge of the N-terminus can be used to strengthen the interaction with a positively charged therapeutic agent.

In some embodiments, the N-terminus domain is altered by the addition of negatively charged amino acids or the elimination of positively charged amino acids so that e.g., Gemcitabine (Cytidine, 2′-deoxy-2′,2′-difluoro-, monohydrochloride, pKₐ 3.58) may become complexed through electrostatic interaction. In certain embodiments, the loading of the drug may be carried out with gemcitabine in HCl-solution, as it is presented in currently available i.v. formulations. In these embodiments, replacement of Arg or Lys residues in the terminal region with Glu or Asp may reduce the overall positive charge on the inner surface of the VLP.

In some embodiments a reduction in the overall positive charge on the inner surface of the VLP, or an introduction of additional negative charges may be used to increase the electrostatic interaction of the VLP with e.g., Cisplatinum, an inorganic and water-soluble platinum complex, carrying a net charge of +2.

In some embodiments, the N-terminus domain is altered by the elimination of positively charged amino acids, or by deletion of the domain, partially or fully, so that e.g., XELODA (capecitabine), which does not carry a net charge may be stabilized and efficiently loaded into the VLP.

In some embodiments, e.g., PACLITAXEL may be loaded into VLPs with neutral charge, wherein the N-terminus domain is altered by the elimination of positively charged amino acids, or by partial or full deletion of the domain. In these embodiments, providing a VLPs with neutral charge in the interior provided by the N-terminus prevents crystal formation and aggregation of PACLITAXEL due to an excess of negative charge in the PACLITAXEL formulation. In some embodiments, Taxol may be loaded into the VLPs in an ethanol-saline formulation, avoiding the need for Cremophor EL® (polyethoxylated castor oil), which is highly toxic.

In some embodiments, the net charge of the N-terminus is altered to increase the hydrophobic interaction between the therapeutic agent and the interior surface of the VLP. For example, one or more residues of amino acids 1-26 of the N-terminus of the coat protein may be modified to carry uncharged hydrophobic residues, e.g., phenylalanine, tyrosine, or tryptophan, to strengthen retention of hydrophobic therapeutic agents that are difficult or impossible to solubilize in aqueous solutions, such as e.g., DOCETAXEL.

In some embodiments, mosaic VLPs are provided comprising two or more different self-assembling coat proteins as described herein (e.g., a wild-type and one or more variant forms, or two or more variant forms without a wild-type form, etc.). In certain embodiments, the self-assembling proteins are CCMV coat proteins. In certain embodiments, mosaic VLP are provided that comprise two or more different wild-type or genetically modified CCMV coat proteins. For example, mosaic VLP may be produced comprising wild-type CCMV coat protein and genetically modified CCMV coat proteins that are modified as described herein, such as, for example modified in the N-terminal domain (e.g., amino acids 1-26) to e.g., strengthen or weaken the electrostatic interaction between a therapeutic agent and the interior surface of the modified VLP, e.g., to enhance loading of the VLP with the therapeutic agent or to enhance delivery of the agent in the target cell. In certain embodiments, the wild-type CCMV coat proteins and genetically modified CCMV coat proteins may be, described herein, may be disassembled in vitro and the two types of subunits may be reassembled together, producing mosaic VLPs comprising genetically modified subunits (e.g., with an altered interior charge as a consequence of mutating one or more charged amino acids of the N-terminal domain, e.g., amino acids 1-26) and unmodified (wild-type) subunits. The ratio between the two types may be varied at will, for example by adding different concentrations of subunits together for reassembly. Mosaic VLP may be produced (reaasembled) that contain just one subunit of one class of CCMV coat protein (e.g., genetically modified) while all other subunits are of the other class (e.g., wild-type) or they may contain equal amounts of subunits (that is about 50% each of the total number of subunits), or nearly equal amounts (e.g., 40% of the total number of subunits of a VLP are genetically modified and 60% are wild-type). It should be appreciated that any ratio can be produced, and the genetically modified subunits may outnumber the wild-type subunits. It should further be appreciated that the invention is not limited to VLPs comprising wild-type and genetically modified subunits. VLPs are also provided that comprise two or more different modified coat proteins. For example, one modified subunit may comprise mutations in the N-terminus (e.g., amino acids 1-26) that alter the interior charge, as described herein. The second modified subunit may comprise an N-terminal deletion (e.g., amino acids 1-26 or 8-26). Mosaic VLP may be produced (reaasembled) that contain mixtures of two or more classes of modified CCMV coat protein. The ratio between the two types may be varied at will.

VLPs are also provided that comprise three or more different modified coat proteins or wild-type coat proteins. For example, VLPs may comprise two different kinds of modified coat proteins and additionally wild-type coat protein. The ratio between the three types may be varied at will.

In some embodiments, VLPs are provided comprising an N-terminal deletion of the coat protein. For example, some coat protein subunits comprising deletions of N-terminal amino acids assemble into T=1 (60 subunits) and T=2 (120 subunits), as well as the wild-type T=3 (180 subunits) particles. In certain embodiments, ratios for two different subunits (e.g., wild-type and modified) that might be desirable are, e.g., for T=3, 1:179, 2:178, 3:177 subunits, until an equal ratio is achieved, e.g., 90:90, and all ratios in between. For T=2, the ratio may be any ratio between 1:119 and 60:60. For T=1, the ratio may be any ratio between 1:59 and 30:30. For three different subunits, the ratios can range from for T=3, 1:1:178, 1:2:177, 1:3:176, 2:2:176, 1:4:175, 2:3:175 subunits etc., until an equal ratio is achieved, e.g., 60:60:60, and all ratios in between. For T=2, the ratio may be any ratio between 1:1:118 and 40:40:40. For T=1, the ratio may be any ratio between 1:1:58 and 20:20:20.

It should be appreciated that the resulting ratio after VLP assembly may not necessarily be the ratio in which the different subunits may be added to the reassembly reaction (reassembly mix). For example, certain subunits may be prone to aggregation and/or precipitation during VLP reassembly. Such subunits may not contribute equally to the resulting VLP since they may not be equally freely accessible in solution for VLP reassembly as non-aggregated/non-precipitated subunits. For example, certain subunits my be sterically (structurally), electrostatically or in other ways thermodynamically disadvantaged during VLP reassembly and integration of these subunits occurs less frequently than expected based on subunit concentration in the reassembly mix in comparison to other subunits (e.g., wild-type subunits). It should be appreciated that because of these and other reasons input ratios may not equal output ratios. Input ratio is the ratio of subunits that are added to a reassembly reaction (reassembly mix). Output ratio is the ratio of subunits in the assembled VLP. For example, a subunit that is prone to precipitation and/or that has been significantly altered sterically, e.g., by insertion of a large targeting peptide or electrostatically, e.g., by altering the charged residues of the N-terminal domain, may have to be added in 2×, 3×, 4×, 5×, 6×, 7×, 8×, 9×, 10×, 15×, 20×, 30×, 40×, 50×, 100×, 1000× excess (e.g., as judged by input coat protein concentration) to contribute equally (e.g., to about 50% for two subunits or about 33% for three subunits) to the resulting VLP.

In some embodiments, the N-terminus is altered to increase the specific incorporation of one or more nucleic acid molecules. In certain embodiments, the wild-type N-terminus of a CCMV or other viral coat protein may be replaced with functional portions of the MS2 coat protein from bacteriophage to increase the interaction of the nucleic acid and the VLP. In these embodiments, the nucleic acids may be fused to a sequence from the hairpin/translational operator (TR) from the bacteriophage MS2 (e.g., the 19 nucleotide sequence from the hairpin/translational operator). In some embodiments, this sequence is derived from the start of the replicase gene of MS2 and interacts specifically with a pocket formed by a dimer of the MS2 coat protein. In these embodiments, the N-terminal replacement of the wild-type N-terminus of the CCMV coat protein with portions of the MS2 coat protein from bacteriophage generates the MS2-derived pocket that specifically interacts with the TR sequence of the nucleic acid to increase specific encapsidation of such nucleic acids. In some embodiments, the modified CCMV coat protein comprises a RNA bacteriophage Qbeta coat protein sequence and the heterologous RNA molecule comprises a sequence from the Qbeta hairpin/translational operator (TR) that interacts with (binds to) the Qbeta coat protein sequence. In certain embodiments, the modified CCMV coat protein comprises a different RNA binding amino acid sequence and the heterologous RNA molecule comprises a motif that binds to this RNA binding amino acid sequence. The RNA motif and RNA binding amino acid sequences may be derived from natural binding molecules or may be synthetic binding partners. For example, translational operators may be derived from other RNA viruses such as other RNA bacteriophages (e.g., Qβ, R17, fr, GA, Sp, Mi I, MXI, NL95, AP205, f2, or PP7) and corresponding RNA binding amino acids from these viruses may be used. In some embodiments, RNA binding amino acids and RNA motifs may be derived from other virus proteins and their packaged RNA (e.g., Hepatitis B core antigen, Human Papiloma virus, human immunodeficiency virus, human influenza virus, etc.).

In certain embodiments, VLP are provided comprising coat protein subunits that lack the N-terminal domain or portions thereof (e.g., amino acids 1-26 or 8-26). The first 25 amino acids face the interior surface of the virion. These 25 amino acids are highly disordered/mobile and are thought to be required for efficient viral RNA packaging. Nine of the first 25 amino acids are basic (Arg, Lys) and are thought to neutralize the negatively charged RNA. The N-terminal 25 amino acids are thought not to be involved in the structural integrity of the virion and are thought not to be required for virion assembly. It was found that deletion of amino acids 8-26 of the N-terminal domain, a region which contains all 8 of the basic residues, but retaining the first 7 N-terminal amino acids (MSTVGTG, SEQ ID NO: 506) generates coat proteins that maintain their ability to self-assemble into VLP that are empty, that is the VLP are devoid of, or essentially devoid of nucleic acids (e.g., viral RNA). In certain embodiments the VLP are further devoid of host cell nucleic acids (e.g., residual RNA and/or DNA originating from the host cell of the expression system, e.g., P. pastoris or E. coli). In certain embodiments, N-terminal deletion mutants are provided that maintain the ability to self-assemble and that assemble into VLP that are essentially devoid of, or devoid of, nucleic acids (e.g., RNA and/or DNA) originating from the virus and/or the host cell. It should be appreciated that the ability to self-assemble (rates of reassembly) can be tested by analysis using e.g., electron microscopy. Assembled particles can be tested for lack of nucleic acids e.g., by agarose gel separation of samples of disrupted VLP (to release any nucleic acid) and staining (after separation) with dyes that interact (e.g., intercalate) with nucleic acids (e.g., ethidium bromide). In certain embodiments, N-terminal deletion mutants are provided that maintain the ability to self-assemble and that assemble to VLP that are essentially devoid of, or devoid of, RNA and/or DNA originating from the virus and/or the host cell for use as drug delivery vehicles. In certain embodiments, the drugs are small molecules.

In some embodiments, mosaic VLPs are provided comprising two or more different wild-type or modified self-assembling CCMV coat proteins. In certain embodiments, mosaic VLP are provided that comprise two or more different wild-type or genetically modified CCMV coat proteins. For example, mosaic VLP may be produced comprising wild-type CCMV coat protein and the N-terminal deletion mutants of the CCMV coat protein (e.g., deletion of amino acids 1-26) to e.g., to produce VLP delivery vehicles that are (essentially) devoid of all viral and non-viral nucleic acids useful for in vivo delivery of small nucleic acid molecules, including antisense nucleic acids and short interfering nucleic acid (siNA), the latter include, for example: microRNA (miRNA), short interfering RNA (siRNA), double-stranded RNA (dsRNA), short hairpin RNA (shRNA) molecules, circular siRNA, hybrid DNA-siRNA (for example crook siRNA). The ratio between the two types of subunits may be varied at will. In certain embodiments, mosaic VLP may be produced (reaasembled) that contain just one subunit of wild-type CCMV coat protein, e.g., to allow binding of siNA molecules to be loaded into the VLP while all other subunits are N-terminal deletion mutants, e.g., to avoid any (contaminating) nucleic acids derived from the virus and/or expression system host cell remaining in the VLP after VLP reassembly. It will be appreciated that other ratios (e.g., of wild-type to N-terminal deletion subunits) are also possible and the invention is not limited in this regard. In some embodiments, the ratio may be 50%:50%, in other embodiments, the ratio may be, for example, 1%:99%, 5%:95%, 10%:90%, 20%:80%, 30%:70%, or 40%:60%. It should further be appreciated that two or more, three or more different subunits may be assembled into VLPs, e.g., subunits that are wild-type, subunits that are N-terminal deletions and subunits that comprise a targeting peptide in one or more of the surface exposed loops. The ratio between the three types of subunits may be varied at will. For example, for three different subunits, the ratios can range from for T=3, 1:1:178, 1:2:177, 1:3:176, 2:2:176, 1:4:175, 2:3:175 subunits etc., until an equal ratio is achieved, e.g., 60:60:60, and all ratios in between. For T=2, the ratio may be any ratio between 1:1:118 and 40:40:40. For T=1, the ratio may be any ratio between 1:1:58 and 20:20:20. It should be appreciated that in some embodiments these ratios may be used as input ratios in a reassembly reaction. However, in some embodiments these ratios may be the desired output ratios from an assembly reaction and slightly different input ratios may be required in order to generate these output ratios as described herein. In any of the assembly or reassembly reactions described herein in the context of VLP or mosaic VLP preparation, the relative ratios of different coat protein preparations may be evaluated using any suitable technique for detecting and/or measuring protein concentrations or amounts. It should be appreciated that in some embodiments the ratios described herein may be approximate and similar or intermediate or higher or lower ratios also may be used.

It should be appreciated that in order to produce empty particles, that are are essentially devoid of, or devoid of, nucleic acids (e.g., RNA and/or DNA) originating from the virus and/or the host cell (i.e. heterologous nucleic acids) it may not be necessary to delete the N-terminal 25 or 26 amino acids or delete amino acids 8-26. For example mutations that increase the negative charge of the N-terminal region (e.g., substitutions of the arginines (R) and or lysines (K) of the N-terminal amino acids 1-26: Lys8, Arg 11, Arg14, Arg15, Arg19, Lys20, Lys22, Arg23, and/or Arg26 with glutamic (D) or aspartic acid (E)) may be generated to avoid or reduce contamination with heterologous nucleic acids (e.g., through electrostatic repulsion).

In certain embodiments, amino acids may be selected in this region (e.g., amino acids 1-26 or 8-26) for mutagenesis (amino acid substitution) that enhance exclusion of heterologous nucleic acids during VLP production and/or assembly thereby producing empty VLP that are are essentially devoid of, or devoid of, heterologous nucleic acids but the substituted N-terminal amino acids still promote interactions (e.g., electrostatic) during packaging or loading of small interfering nucleic acids (e.g., siRNAs or miRNAs), as described herein. These amino acid substitutions can be determined for example by mutagenesis screening of the N-terminal region, e.g., amino acids 1-26 or 8-26 using standard laboratory protocols and techniques.

In certain embodiments, mosaic VLP are provided comprising CCMV coat proteins comprising a N-terminal replacement of the wild-type N-terminus with portions of the MS2 coat protein from bacteriophage to generate the MS2-derived pocket that specifically interacts with the TR sequence of the nucleic acid to increase specific encapsidation of such nucleic acids. In certain embodiments, mosaic VLP are provided comprising CCMV coat proteins comprising a N-terminal replacement of the wild-type N-terminus with portions of the MS2 coat protein from bacteriophage the mosaic VLP further comprising a N-terminal deletion mutant of the CCMV coat protein.

Nucleic acids that may be used (e.g., packaged) in connection with VLPs and mosaic VLPs can be DNA and/or RNA molecules. In some aspects, the invention relates to the use of small nucleic acid molecules, including antisense nucleic acids and short interfering nucleic acid (siNA), the latter include, for example: microRNA (miRNA), short interfering RNA (siRNA), double-stranded RNA (dsRNA), and short hairpin RNA (shRNA) molecules to knockdown expression of target genes associated with a disease or disorder. An siNA of the invention can be unmodified or chemically-modified. An siNA of the instant invention can be chemically synthesized, expressed from a vector or enzymatically synthesized. The instant invention also features various chemically-modified synthetic short interfering nucleic acid (siNA) molecules capable of modulating gene expression or activity in cells by RNA interference (RNAi). The use of chemically-modified siNA improves various properties of native siNA molecules through, for example, increased resistance to nuclease degradation in vivo and/or through improved cellular uptake. Furthermore, siNA having multiple chemical modifications may retain its RNAi activity. The siNA molecules of the instant invention provide useful reagents and methods for a variety of therapeutic applications.

Chemically synthesizing nucleic acid molecules with modifications (base, sugar and/or phosphate) that prevent their degradation by serum ribonucleases can increase their potency (see e.g., Eckstein et al., International Publication No. WO 92/07065; Perrault et al, 1990 Nature 344, 565; Pieken et al., 1991, Science 253, 314; Usman and Cedergren, 1992, Trends in Biochem. Sci. 17, 334; Usman et al., International Publication No. WO 93/15187; and Rossi et al., International Publication No. WO 91/03162; Sproat, U.S. Pat. No. 5,334,711; and Burgin et al., supra; all of these describe various chemical modifications that can be made to the base, phosphate and/or sugar moieties of the nucleic acid molecules herein). In some embodiments, modifications which enhance their efficacy in cells, and removal of bases from nucleic acid molecules to shorten oligonucleotide synthesis times and reduce chemical requirements are desired.

There are several examples in the art describing sugar, base and phosphate modifications that can be introduced into nucleic acid molecules with significant enhancement in their nuclease stability and efficacy. For example, oligonucleotides can be modified to enhance stability and/or enhance biological activity by modification with nuclease resistant groups, for example, 2′ amino, 2′-C-allyl, 2′-fluoro, 2′-O-methyl, 2′-H, nucleotide base modifications (for a review see Usman and Cedergren, 1992, TIBS. 17, 34; Usman et al., 1994, Nucleic Acids Symp. Ser. 31, 163; Burgin et al., 1996, Biochemistry, 35, 14090). Sugar modification of nucleic acid molecules has been extensively described in the art (see Eckstein et al., International Publication PCT No. WO 92/07065; Perrault et al. Nature, 1990, 344, 565 568; Pieken et al. Science, 1991, 253, 314317; Usman and Cedergren, Trends in Biochem. Sci., 1992, 17, 334 339; Usman et al. International Publication PCT No. WO 93/15187; Sproat, U.S. Pat. No. 5,334,711 and Beigelman et al., 1995, J. Biol. Chem., 270, 25702; Beigelman et al., International PCT publication No. WO 97/26270; Beigelman et al., U.S. Pat. No. 5,716,824; Usman et al.). In some embodiments, a molecule comprises one or more chemical modifications.

In some embodiments, one of the strands of the double-stranded siNA molecule comprises a nucleotide sequence that is complementary to a nucleotide sequence of a target RNA or a portion thereof, and the second strand of the double-stranded siNA molecule comprises a nucleotide sequence identical to the nucleotide sequence or a portion thereof of the targeted RNA. In another embodiment, one of the strands of the double-stranded siNA molecule comprises a nucleotide sequence that is substantially complementary to a nucleotide sequence of a target RNA or a portion thereof, and the second strand of the double-stranded siNA molecule comprises a nucleotide sequence substantially similar to the nucleotide sequence or a portion thereof of the target RNA. In another embodiment, each strand of the siNA molecule comprises about 19 to about 23 nucleotides, and each strand comprises at least about 19 nucleotides that are complementary to the nucleotides of the other strand.

In some embodiments an siRNA is an shRNA, shRNA-mir, or microRNA molecule encoded by and expressed from a genomically integrated transgene or a plasmid-based expression vector. Thus, in some embodiments a molecule capable of inhibiting mRNA expression, or microRNA activity, is a transgene or plasmid-based expression vector that encodes a small-interfering nucleic acid. Such transgenes and expression vectors can employ either polymerase II or polymerase III promoters to drive expression of these shRNAs and result in functional siRNAs in cells. The former polymerase permits the use of classic protein expression strategies, including inducible and tissue-specific expression systems. In some embodiments, transgenes and expression vectors are controlled by tissue specific promoters. In certain embodiments transgenes and expression vectors are controlled by inducible promoters, such as tetracycline inducible expression systems.

In some embodiments, a small interfering nucleic acid of the invention is expressed in mammalian cells using a mammalian expression vector. The recombinant mammalian expression vector may be capable of directing expression of the nucleic acid preferentially in a particular cell type (e.g., tissue-specific regulatory elements are used to express the nucleic acid). Tissue specific regulatory elements are known in the art. Non-limiting examples of suitable tissue-specific promoters include the myosin heavy chain promoter, albumin promoter, lymphoid-specific promoters, neuron specific promoters, pancreas specific promoters, and mammary gland specific promoters. Developmentally-regulated promoters are also encompassed, for example the murine hox promoters and the a-fetoprotein promoter.

siRNA molecules are well know in the art and many siRNAs are known that target tumor-specific proteins that may be mutated, overexpressed and/or deregulated, such as for example cyclin/cdk, EGFR, brc/abl and the like.

Accordingly, aspects of the invention can be used to deliver molecules that promote RNA interference using any of a variety of molecules known in the art, e.g., short interfering RNA molecules (siRNA), which are double stranded RNA molecules. As described herein, RNA interference (RNAi) is a phenomenon describing double-stranded (ds)RNA-dependent gene specific posttranscriptional silencing. Synthetic duplexes of 21 nucleotide RNAs can mediate gene specific RNAi in mammalian cells, without invoking generic antiviral defense mechanisms (Elbashir et al. Nature 2001, 411:494-498; Caplen et al. PNAS 2001, 98:9742-9747).

In some embodiments, polynucleotides are provided comprising an RNAi sequence that acts through an RNAi mechanism to attenuate or inhibit expression of a gene of interest, e.g., a gene that is overexpressed in cancer. In some embodiments, the siRNA sequence is between about 19 nucleotides and about 75 nucleotides in length, or between about 25 base pairs and about 35 base pairs in length. An RNAi construct contains a nucleotide sequence that hybridizes under physiologic conditions of the cell to the nucleotide sequence of at least a portion of the mRNA transcript of a gene of interest. In certain embodiments, the double-stranded RNA need only be sufficiently similar to natural RNA that it has the ability to mediate RNAi. In certain embodiments, the number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

In certain embodiments, sequence identity may be optimized by sequence comparison and alignment algorithms known in the art (see Gribskov and Devereux, Sequence Analysis Primer, Stockton Press, 1991) and calculating the percent difference between the nucleotide sequences by, for example, the Smith-Waterman algorithm as implemented in the BESTFIT software program using default parameters (e.g., University of Wisconsin Genetic Computing Group). In certain embodiments, the sequence identity between the inhibitory RNA and the portion of the target gene is greater than 85%, 86%, 87%, 88%, 89%, 90%, 91%, 92%, 93%, 94%, 95%, 96%, 97%, 98%, 99%, or is 100%.

Production of polynucleotides comprising RNAi sequences is well known in the art. For example, polynucleotides comprising RNAi sequences can be produced by chemical synthetic methods or by recombinant nucleic acid techniques. Endogenous RNA polymerase of the treated cell may mediate transcription in vivo, or cloned RNA polymerase can be used for transcription in vitro. In certain embodiments, the polynucleotides that modulate target gene activity by RNAi mechanisms, may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. In certain embodiments, the siRNA polynucleotides may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “siRNAs.” These nucleic acids are between about 19-35 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex or translation is inhibited. In a particular embodiment, the 21-23 nucleotides siRNA molecules comprise a 3′ hydroxyl group.

The siRNA molecules can be purified using a number of techniques known to those of skill in the art. For example, gel electrophoresis can be used to purify such molecules. Alternatively, non-denaturing methods, such as non-denaturing column chromatography, can be used to purify the siRNA molecules. In addition, chromatography (e.g., size exclusion chromatography), glycerol gradient centrifugation, affinity purification with antibody can be used to purify siRNAs.

In certain embodiments, at least one strand of the siRNA sequence of an effector domain has a 3′ overhang from about 1 to about 6 nucleotides in length, or from 2 to 4 nucleotides in length. In other embodiments, the 3′ overhangs are 1-3 nucleotides in length. In certain embodiments, one strand has a 3′ overhang and the other strand is either blunt-ended or also has an overhang. The length of the overhangs may be the same or different for each strand. In order to further enhance the stability of the siRNA sequence, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythymidine is tolerated and does not affect the efficiency of RNAi. The absence of a 2′ hydroxyl significantly enhances the nuclease resistance of the overhang in tissue culture medium and may be beneficial in vivo.

Examples of siRNA that can be delivered according to aspects of the invention include siRNA that mediate silencing of nuclear factor erythroid-2-related factor 2 gene expression in non-small cell lung cancer (Singh et al., Cancer Research 68, 7975-7984, Oct. 1, 2008); anti-chlesterolemic siRNA (Frank-Kamenetsky et al., PNAS, Aug. 19, 2008, vol. 105, no. 33, pp 11915-11920); ophthalmic siRNA; or any other siRNA as the invention is non limited in this respect.

Tools for design and quality of siRNAs, shRNAs and/or miRNAs are known in the art. Web-based online software system for designing siRNA sequences and scrambled siRNA sequences are for example siDirect, siSearch, SEQ2SVM, Deqor, siRNA Wizard (InvivoGen). The specificity can be predicted using for example SpecificityServer, miRacle. Target sequences can be researched for example at HuSiDa (Human siRNA Database), and siRNAdb (a database of siRNA sequences). Exemplary sequences that may be used to target specific proteins are listed in Table 1. It should be appreciated that these sequences may be modified as described herein to include a sequence motif that binds to an RNA binding amino acid sequence that may be incorporated into a modified coat protein of a VLP as described herein (e.g., in the amino terminal region). In some embodiments, the sequences listed in Table 1 may be used to design RNA or DNA molecules (e.g., siRNA, antisense, etc.). Accordingly, the sequences listed as RNA or DNA may be used to design corresponding DNA or RNA molecules, respectively. In some embodiments, DNA or RNA molecules may have the sequences listed in Table 1 or the complements thereof. In some embodiments, DNA or RNA molecules having any of the sequences described herein may be used as therapeutic agents. In some embodiments, the sequences may be incorporated into longer DNA or RNA molecules.

TABLE 1
siNA sequences

| Accession # | Unigene # | Gene Symbol | Name | Sequence |
|---|---|---|---|---|
| NM_003467 | Hs.421986 | CXCR4 | Chemokine (C-X-C motif) | 5′-UAAAAUCUUCCUGCCCACC-3′ |
| | | | receptor 4 | (SEQ ID NO: 23) |
| NM_003467 | Hs.421986 | CXCR4 | Chemokine (C-X-C motif) | 5′-GGAAGCUGUUGGCUGAAAA-3′ |
| | | | receptor 4 | (SEQ ID NO: 24) |
| NM_006799.2 | Hs.72026 | RSS21 | Protease, serine, 21 | 5′-CACAUCCAGCCCAUCUGUC-3′ |
| | | | (testisin) | (SEQ ID NO: 25) |
| NM_000117.1 | Hs.522823 | EMD | Emerin | 5′-CCGUGCUCCUGGGGCUGGG-3′ |
| | | | | (SEQ ID NO: 26) |
| NM_001350.3 | Hs.336916 | DAXX | Death-associated protein 6 | 5′-GGAGUUGGAUCUCUCAGAA-3′ |
| | | | | (SEQ ID NO: 27) |
| NM_003014.2 | Hs.105700 | SFRP4 | Secreted frizzled-related | 5′-AAGTCCCGCTCATTACAAA-3′ |
| | | | protein 4 | (SEQ ID NO: 28) |
| NM_015062.3 | Hs.533551 | PPRC1 | Peroxisome proliferative | 5′-AAGACCAGCCUCUUUGCCCAG-3′ |
| | | | activated receptor, gamma, | (SEQ ID NO: 29) |
| | | | coactivator-related 1 | |
| NM_001005360.1 | Hs.211463 | DNM2 | Dynamin 2 | 5′-GGACCAGGCAGAAAACGAG-3 |
| | | | | (SEQ ID NO: 30) |
| NM_001904.2 | Hs.476018 | CTNNB1 | Catenin (cadherin-associated | 5′-CUAUCAGGAUGACGCGG-3′ |
| | | | protein), beta 1, 88 kDa | (SEQ ID NO: 31) |
| NM_153831.2 | Hs.395482 | PTK2 | PTK2 protein tyrosine | 5′-AACCACCUGGGCCAGUAUUAU-3′ |
| | | | kinase 2 | (SEQ ID NO: 32) |
| NM_001429.2 | Hs.517517 | EP300 | E1A binding protein p300 | 5′-UGACACAGGCAGGCUUGACUU-3′ |
| | | | | (SEQ ID NO: 33) |
| NM_005904.2 | Hs.465087 | SMAD7 | SMAD, mothers against DPP | 5′-AAGCUCAAUUCGGACAACAAG-3′ |
| | | | homolog 7 (Drosophila) | (SEQ ID NO: 34) |
| NM_001904.2 | Hs.476018 | CTNNB1 | Catenin (cadherin-associated | 5′-AAGUCCUGUAUGAGUGGGAAC-3′ |
| | | | protein), beta 1 88 kDa | (SEQ ID NO: 35) |
| NM_75847.1 | Hs.172550 | PTBP1 | Polypyrimidine tract binding | 5′-TCGACGAACATCTACAACGCCTGCTTC |
| | | | protein 1 | AAGAGAGCAGGCGTTGTAGATGTTCTTTTT |
| | | | | TT-3′ |
| | | | | (SEQ ID NO: 36) |
| NM_175847.1 | Hs.172550 | PTBP1 | Polypyrimidine tract binding | 5′-TCGACCAATGACAAGAGCCGTGACTTC |
| | | | protein 1 | AAGAGAGTCACGGCTCTTGTCATTGTTTTT |
| | | | | TT-3′ |
| | | | | (SEQ ID NO: 37) |
| NM_002659.2 | Hs.466871 | PLAUR | Plasminogen activator, | 5′-GGTGAAGAAGGGCGTCCAA-3′ |
| | | | urokinase receptor | (SEQ ID NO: 38) |
| NM_033360.2 | Hs.505033 | KRAS2 | V-Ki-ras2 Kirsten | 5′-GATCCGTTGGAGCTGTTGGCGTAG |
| | | | rat sarcoma | TTCAAGAGACTACGCCAACAGCTCCA |
| | | | 2 viral oncogene homolog | ACTTTTTGGAAA-3′ |
| | | | | (SEQ ID NO: 39) |
| NM_002959.4 | Hs.485195 | S0RT1 | Sortilin 1 | 5′-AGGTGGTGTTAACAGCAGAG-3′ |
| | | | | (SEQ ID NO: 40) |
| NM_002959.4 | Hs.485195 | SORT1 | Sortilin 1 | 5′-AATGTTCCAATGCCCCACTC-3′ |
| | | | | (SEQ ID NO: 41) |
| NM_000743.2 | Hs.89605 | CKRNA3 | Cholinergic receptor, | 5′-AACUGCCAGUGGCCAGGGCCU-3′ |
| | | | nicotinic, alpha polypeptide | (SEQ ID NO: 42) |
| | | | 3 | |
| NM_004859.2 | Hs.491351 | CLTC | Clathrin, heavy polypeptide | 5′-AACCUGCGGUCUGGAGUCAAC-3′ |
| | | | (Hc) | (SEQ ID NO: 43) |
| NM_004859.2 | Hs.491351 | CLTC | Clathrin, heavy polypeptide | 5′-UAAUCCAAUUCGAAGACCAAU-3′ |
| | | | (Hc) | (SEQ ID NO: 44) |
| NM_000038.3 | Hs.158932 | APC | Adenomatosis polyposis coli | 5′-AGGGGCAGCAACTGATGAAAA-3′ |
| | | | | (SEQ ID NO: 45) |
| NM_004850.3 | Hs.58617 | ROCK2 | Rho-associated, coiled-coil | 5′-AAGGCATCGCAGAAGGTTTAT-3′ |
| | | | containing protein kinase 2 | (SEQ ID NO: 46) |
| NM_001274.2 | Hs.24529 | CHEK1 | CHK1 checkpoint homolog | 5′-UCGAAGUACUCAGCGUAAG-3′ |
| | | | (S. pombe) | (SEQ ID NO: 47) |
| NM_007194.3 | Hs.291363 | CHEK2 | CHK2 checkpoint homolog | 5′-GAACCUGAGGACCAAGAAC-3′ |
| | | | (S. pombe) | (SEQ ID NO: 48) |
| NM_001901.1 | Hs.410037 | CTGF | Connective tissue growth | 5′-AATGTTCTCTTCCAGGTCAGCCCTGT |
| | | | factor | CTC-3′ |
| | | | | (SEQ ID NO: 49) |
| NM_001619.2 | Hs.83636 | ADRBK1 | Adrenergic, beta, receptor | 5′-AAGAAGUACGAGAAGCUGGAG-3′ |
| | | | kinase 1 | (SEQ ID NO: 50) |
| NM_005160.2 | Hs.517493 | ADRBK2 | Adrenergic, beta, receptor | 5′-AAGCAAGCUGUAGAACACGUA-3′ |
| | | | kinase 2 | (SEQ ID NO: 51) |
| NM_005308.2 | Hs.524625 | GRK5 | G protein-coupled receptor | 5′-AAGCCGUGCAAAGAACUCUUU-3′ |
| | | | kinase 5 | (SEQ ID NO: 52) |
| NM_001004106.1 | Hs.235116 | GRK6 | G protein-coupled receptor | 5-AACAGUAGGUUUGUAGUGAGC-3′ |
| | | | kinase 6 | (SEQ ID NO: 53) |
| NM_017556.1 | Hs.530101 | FBLP-1 | Filamin-binding LIM | 5′-AAAGGGGCAUCCACAGACAUC-3′ |
| | | | protein-1 | (SEQ ID NO: 54) |
| NM_005857.2 | Hs.132642 | ZMPSTE24 | Zinc metallopeptidase | 5′-TTATTCTTCTCTTTGGAGGA-3′ |
| | | | (STE24 homolog, yeast) | (SEQ ID NO: 55) |
| NM_005572 | Hs.491359 | LMNA | Lamin A/C | 5′-ACTGGACTTCCAGAAGAAC-3′ |
| | | | | (SEQ ID NO: 56) |
| NM_015878.3 | Hs.459106 | OAZIN | Ornithine decarboxylase | 5′-AATTGCACGTAATCACCCAAA-3′ |
| | | | antizyme inhibitor | (SEQ ID NO: 57) |
| NM_015878.3 | Hs.459106 | OAZIN | Ornithine decarboxylase | 5′-AAGAAATACAAGGAAGATGAG-3′ |
| | | | antizyme inhibitor | (SEQ ID NO: 58) |
| NM_001664.2 | Hs.247077 | RHOA | Ras homolog gene family, | 5′-GACAUGCUUGCUCAUAGUC-3′ |
| | | | member A | (SEQ ID NO: 59) |
| NM_175744.3 | Hs.502659 | RHOC | Ras homolog gene family, | 5′-GACCUGCCUCCUCAUCGUC-3′ |
| | | | member C | (SEQ ID NO: 60) |
| NM_000041.2 | Hs.515465 | APOE | Apolipoprotein E | 5′-AAGGTGGAGCAAGCGGTGGAG-3′ |
| | | | | (SEQ ID NO: 61) |
| NM_000041.2 | Hs.515465 | APOE | Apolipoprotein E | 5′-AAGGAGTTGAAGGCCTACAAA-3′ |
| | | | | (SEQ ID NO: 62) |
| AF520590.1 | Hs.536600 | BAK1 | BCL2-antagonist/killer 1 | 5′-UGCCUACGAACUCUUCACC-3′ |
| | | | | (SEQ ID NO: 63) |
| NM_138761.2 | Hs.159428 | BAX | BCL2-associated X protein | 5′-UAUGGAGCUGCAGAGGAUG-3′ |
| | | | | (SEQ ID NO: 64) |
| NM_005733.1 | Hs.73625 | KIF20A | Kinesin family member 20A | 5′-TTGGCCAAGCCACACACAG-3′ |
| | | | | (SEQ ID NO: 65) |
| NM_005733.1 | Hs.73625 | KIF20A | Kinesin family member 20A | 5′-GTTCTCAGCCATTGCTAGC-3′ |
| | | | | (SEQ ID NO: 66) |
| NM_005733.1 | Hs.73625 | KIF20A | Kinesin family member 20A | 5′-GGCAGCATGTATTGCTGAG-3′ |
| | | | | (SEQ ID NO: 67) |
| NM_014034.1 | Hs.292316 | ASF1A | ASF1 anti-silencing function 1 | 5′-AAUC CAGGACUCAUUCCAGAU-3′ |
| | | | homolog A (S. cerevisiae) | (SEQ ID NO: 68) |
| NM_014034.1 | Hs.292316 | ASF1A | ASF1 anti-silencing function 1 | 5′-AAGUGAAGAAUACGAUCAAGU-3′ |
| | | | homolog A (S. cerevisiae) | (SEQ ID NO: 69) |
| NM_018154.1 | Hs.26516 | ASF1B | ASF1 anti-silencing function 1 | 5′-AACAACGAGUACCUCAACCCU-3′ |
| | | | homolog B (S. cerevisiae) | (SEQ ID NO: 70) |
| NM_022110.3 | Hs.520042 | WISp39 | FK506 binding protein like | 5′-AACGCUUGAGCUGGAAGUAAG-3′ |
| | | | | (SEQ ID NO: 71) |
| NM_022110.3 | Hs.520042 | WISp39 | FK506 binding protein like | 5′-CCUUCAAGCUUCUGAUCUC-3′ |
| | | | | (SEQ ID NO: 72) |
| NM_000389.2 | Hs.370771 | CDKN1A | Cyclin-dependent kinase | 5′-AACUUCGACUUUGUCACCGAG-3′ |
| | | | inhibitor 1A (p21, Kip1) | (SEQ ID NO: 74) |
| NM_004064.2 | Hs.238990 | CDKN1B | Cyclin-dependent kinase | 5′-AAGCACUGCAGAGACAUGGAAG-3′ |
| | | | inhibitor 1B (p27, Kip1) | (SEQ ID NO: 74) |
| NM_033084.2 | Hs.208388 | FANCD2 | Fanconi anemia, | 5′-AACAGCCATGGATACACTTGA-3′ |
| | | | complementation group D2 | (SEQ ID NO: 75) |
| NM_001641.2 | Hs.73722 | APEX1 | APEX nuclease (multifunctional | 5′-AATGACAAAGAGGCAGCAGG-3′ |
| | | | DNA repair enzyme) 1 | (SEQ ID NO: 76) |
| NM_001641.2 | Hs.73722 | APEX1 | APEX nuclease (multifunctional | 5′-AACCTGCCACACTCAAGATC-3′ |
| | | | DNA repair enzyme) 1 | (SEQ ID NO: 77) |
| NM_001641.2 | Hs.73722 | APEX1 | APEX nuclease (multifunctional | 5′-AGCTGAACTTCAGGAGCTGCC-3′ |
| | | | DNA repair enzyme) 1 | (SEQ ID NO: 78) |
| NM_001641.2 | Hs.73722 | APEX1 | APEX nuclease (multifunctional | 5′-AAGCCTTTCGCAAGTTCCTGA-3′ |
| | | | DNA repair enzyme) 1 | (SEQ ID NO: 79) |
| NM_001641.2 | Hs.73722 | APEX1 | APEX nuclease (multifunctional | 5′-ACGGCATAGGCGATGAGGAG-3′ |
| | | | DNA repair enzyme) 1 | (SEQ ID NO: 80) |
| NM_001641.2 | Hs.73722 | APEX1 | APEX nuclease (multifunctional | 5′-AGGAAGGCCGGGTGATTGTG-3′ |
| | | | DNA repair enzyme) 1 | (SEQ ID NO: 81) |
| NM_001641.2 | Hs.73722 | APEX1 | APEX nuclease (multifunctional | 5′-GTCTGGTACGACTGGAGTA-3′ |
| | | | DNA repair enzyme) 1 | (SEQ ID NO: 82) |
| NM_001641.2 | Hs.73722 | APEX1 | APEX nuclease (multifunctional | 5′-GACAGCTTTAGGCACCTCTA-3′ |
| | | | DNA repair enzyme) 1 | (SEQ ID NO: 83) |
| NM_015641.2 | Hs.533391 | TES | Testis derived transcript | 5′-GGAUUCGAACUGCACUUCU-3′ |
| | | | (3 LIM domains) | (SEQ ID NO: 84) |
| NM_015641.2 | Hs.533391 | TES | Testis derived transcript | 5′-ACUGUGGCACCCAGCUUGU-3′ |
| | | | (3 LIM domains) | (SEQ ID NO: 85) |
| NM_003461.3 | Hs.490415 | ZYX | Zyxin | 5′-GCCCAAAGUGAAUCCCUUC-3′ |
| | | | | (SEQ ID NO: 86) |
| NM_002880.2 | Hs.159130 | RAF1 | V-raf-1 murine leukemia viral | 5′-TTTGAATATCTGTGCTGAGAACACAG |
| | | | oncogene homolog 1 | TTCTCAGCACAGATATTCTTT-3′ |
| | | | | (SEQ ID NO: 87) |
| NM_002880.2 | Hs.159130 | RAF1 | N-raf-1 murine leukemia viral | 5′-TTTGTCAATTAGCTGGAACATCACAG |
| | | | oncogene homolog I | ATGTT CCAGCTAATTGACTTTTT-3′ |
| | | | | (SEQ ID NO: 88) |
| NM_004506.2 | Hs.158195 | HSF2 | Heat shock transcription | 5′-AATGAGAAAAGCAAAAGGTGCCCTG |
| | | | factor 2 | TCTC-3′ |
| | | | | (SEQ ID NO: 89) |
| NM_005356.2 | Hs.470627 | LCK | Lymphocyte-specific protein | 5′-CAUCGAUGUGUGUGAGAACUGC-3′ |
| | | | tyrosine kinase | (SEQ ID NO: 90) |
| NM_005546.3 | Hs.483938 | ITK | IL2-inducible T-cell kinase | 5′-CUGUUCUCAGCUGGAGAAGCUU-3′ |
| | | | | (SEQ ID NO: 91) |
| NM_005546.3 | Hs.483938 | ITK | IL2-inducible T-cell kinase | 5′-GGAGCCUUCAUGGUAAGGGAUU-3′ |
| | | | | (SEQ ID NO: 92) |
| NM_002133.1 | Hs.517581 | HMOX1 | Heme oxygenase (decycling) 1 | 5′-GGCACCATGAAGGCG-3′ |
| | | | | (SEQ ID NO: 93) |
| NM_000639.1 | Hs.2007 | FASLG | Tumor necrosis factor (ligand) | 5′-CUGGGCUGUACUUUGUAUA-3′ |
| | | | superfamily, member 6 | (SEQ ID NO: 94) |
| NM_018417.2 | Hs.320892 | SAC | Testicular soluble adenylyl | 5′-AUGUAGCCUGGAGAUCCAUUU-3′ |
| | | | cyclase | (SEQ ID NO: 95) |
| NM_003743.3 | Hs.412293 | NCOA1 | Nuclear receptor coactivator 1 | 5′-CCUCAGGGCAGAGAACCAUCU-3′ |
| | | | | (SEQ ID NO: 96) |
| NM_005572.2 | Hs.491359 | LMNA | Lamin A/C | 5′-CUGGACUUCCAGAAGAACAUC-3′ |
| | | | | (SEQ ID NO: 97) |
| NM_176871.2 | Hs.521444 | PDLIM2 | PDZ and LIM domain 2 | 5′-AAGAUCCGCCAGAGCCCCUCG-3′ |
| | | | (mystique) | (SEQ ID NO: 98) |
| NM_014188.2 | Hs.30026 | HSPC182 | HSPC182 protein | 5′-AACAGGGACTCACGTGAAGCT-3′ |
| | | | | (SEQ ID NO: 99) |
| NM_014188.2 | Hs.30026 | HSPC182 | HSPC182 protein | 5′-AAGACCTGTTTGATCTGATCC-3′ |
| | | | | (SEQ ID NO: 100) |
| AF263744.1 | Hs.519346 | ERBB2IP | Erbb2 interacting protein | 5′-UAGACUGACCCAGCUGGAA-3′ |
| | | | | (SEQ ID NO: 101) |
| NM_002583.2 | Hs.406074 | PAWR | PRKC, apoptosis, WT1, | 5′-GAUGCAAUUACACAACAGA-3′ |
| | | | regulator | (SEQ ID NO: 102) |
| NM_003766.2 | Hs.12272 | BECN1 | Beclin 1 (coiled-coil, myosin- | 5′-CUCAGGAGAGGAGCCAUUU-3′ |
| | | | like BCL2 interacting protein) | (SEQ ID NO: 103) |
| NM_003766.2 | Hs.12272 | BECN1 | Beclin 1 (coiled-coil, myosin- | 5′-GAUUGAAGACACAGGAGGC-3′ |
| | | | like BCL2 interacting protein) | (SEQ ID NO: 104) |
| NM_004849.1 | Hs.486063 | APG5L | APG5 autophagy 5-like | 5′-GCAACUCUGGAUGGGAUUG-3′ |
| | | | (S. cerevisiae) | (SEQ ID NO: 105) |
| NM_031482.3 | Hs.527193 | APG10L | APG10 autophagy 10-like | 5′-GGAGUUCAUGAGUGCUAUA-3′ |
| | | | (S. cerevisiae) | (SEQ ID NO: 106) |
| NM_004707.2 | Hs.264482 | APG12L | APG12 autophagy 12-like | 5′-CAGAGGAACCUGCUGGCGA-3′ |
| | | | (S. cerevisiae) | (SEQ ID NO: 107) |
| NM_002613.2 | Hs.459691 | PDPK1 | 3-phosphoinositide dependent | 5′-AACTGGCAACCTCCAGAGAAT-3′ |
| | | | protein kinase-1 | (SEQ ID NO: 108) |
| NM_002613.2 | Hs.459691 | PDPK1 | 3-phosphoinositide dependent | 5′-AAGAGACCTCGTGGAGAAACT-3′ |
| | | | protein kinase-1 | (SEQ ID NO: 109) |
| NM_000314.2 | Hs.500466 | PTEN | Phosphatase and tensin homolog | 5′-AACAGTAGAGGAGCCGTCAAA-3′ |
| | | | (mutated in multiple advanced | (SEQ ID NO: 110) |
| | | | cancers 1) | |
| NM_006092.1 | Hs.405153 | CARD4 | Caspase recruitment domain | 5′-GGGUGAGACCAUCUUCAUC-3′ |
| | | | family, member 4 | (SEQ ID NO: 111) |
| NM_006092.1 | Hs.405153 | CARD4 | Caspase recruitment domain | 5′-GGCCAAAGUCUAUGAAGAU-3′ |
| | | | family, member 4 | (SEQ ID NO: 112) |
| NM_000598.3 | Hs.450230 | IGFBP3 | Insulin-like growth factor | 5′-AAUCAUCAUCAAGAAAGGGCA-3′ |
| | | | binding protein 3 | (SEQ ID NO: 113) |
| NM_006839.1 | Hs.148559 | IMMT | Inner membrane protein, | 5′-AAUUGCUGGAGCUGGCCUU-3′ |
| | | | mitochondrial (mitofilin) | (SEQ ID NO: 114) |
| NM_016485.3 | Hs.431367 | C6ORF55 | Chromosome 6 open reading | 5′-GAATGAAGATCGATAGTAA-3′ |
| | | | frame 55 | (SEQ ID NO: 115) |
| NM_016485.3 | Hs.431367 | C6ORF55 | Chromosome 6 open reading | 5′-GCACAGGTGTAGCAAGTAA-3′ |
| | | | frame 55 | (SEQ ID NO: 116) |
| NM_016485.3 | Hs.431367 | C6ORF55 | Chromosome 6 open reading | 5′-GGAGAATTATGCTTTGAAA-3′ |
| | | | frame 55 | (SEQ ID NO: 117) |
| NM_016485.3 | Hs.431367 | C6ORF55 | Chromosome 6 open reading | 5′-GCAGTGCTTTGCAGTATGA-3′ |
| | | | frame 55 | (SEQ ID NO: 118) |
| NM_016410.2 | Hs.415534 | SNF7DC2 | SNF7 domain containing 2 | 5′-CAGAAAGCCTTGCGAGTTT-3′ |
| | | | | (SEQ ID NO: 119) |
| NM_016410.2 | Hs.415534 | SNF7DC2 | SNF7 domain containing 2 | 5′-GAATTTGGATTGCCACAGA-3′ |
| | | | | (SEQ ID NO: 120) |
| NM_016410.2 | Hs.415534 | SNF7DC2 | SNF7 domain containing 2 | 5′-GAAGGTGTTCCCACTGATA-3′ |
| | | | | (SEQ ID NO: 121) |
| NM_016410.2 | Hs.415534 | SNF7DC2 | SNF7 domain containing 2 | 5′-GAGAGGGTCCTGCAAAGAA-3′ |
| | | | | (SEQ ID NO: 122) |
| NM_199185.1 | Hs.519452 | NPM1 | Nucleophosmin (nucleolar | 5′-UGAUGAAAAUGAGCACCAG-3′ |
| | | | phosphoprotein B23, numatrin) | (SEQ ID NO: 123) |
| NM_003118.2 | Hs.111779 | SPARC | Secreted protein, acidic, | 5′-AAAATCCCTGCCAGAACCACC-3′ |
| | | | cysteine-rich | (SEQ ID NO: 124) |
| NM_003118.2 | Hs.111779 | SPARC | Secreted protein, acidic, | 5′-AACAAGACCTTCGACTCTTCC-3′ |
| | | | cysteine-rich | (SEQ ID NO: 125) |
| NM_003183.3 | Hs.404914 | ADAM17 | A disintegrin and | 5′-AAACGAAAGCGAGTACACT-3′ |
| | | | metalloproteinase domain 17 | (SEQ ID NO: 126) |
| | | | (tumor necrosis factor, alpha, | |
| | | | converting enzyme) | |
| NM_012164.2 | Hs.494985 | FBXW2 | F-box and WD-40 domain | 5′-AGATGGACTTCTCTGTACAGG-3′ |
| | | | protein 2 | (SEQ ID NO: 127) |
| NM_012164.2 | Hs.494985 | FBXW2 | F-box and WD-40 domain | 5′-GACATTGTCTGTCTCTGAGGA-3′ |
| | | | protein 2 | (SEQ ID NO: 128) |
| NM_175940.1 | Hs.272813 | DUOX1 | Dual oxidase 1 | 5′-GGACUUAUCCUGGCUAGAG-3′ |
| | | | | (SEQ ID NO: 129) |
| NM_004503.2 | Hs.820 | HOXC6 | Homeo box C6 | 5′-CCGGAUCUACUCGACUCCC-3′ |
| | | | | (SEQ ID NO: 130) |
| NM_004503.2 | Hs.820 | HOXC6 | Homeo box C6 | 5′-CCUAAUCACACACUCUGUA-3′ |
| | | | | (SEQ ID NO: 131) |
| NM_004503.2 | Hs.820 | HOXC6 | Homeo box C6 | 5′-ACUGCAGACAAAACACCUU-3′ |
| | | | | (SEQ ID NO: 132) |
| NM_004503.2 | Hs.820 | HOXC6 | Homeo box C6 | 5′-UCCAACCUCUGGGUCCGUU-3′ |
| | | | | (SEQ ID NO: 133) |
| NM_004503.2 | Hs.820 | HOXC6 | Homeo box C6 | 5′-ACUGUGACCGUUUCUGUGU-3′ |
| | | | | (SEQ ID NO: 134) |
| NM_004503.2 | Hs.820 | HOXC6 | Homeo box C6 | 5′-CUCAGACUCUACAGAUUGC-3′ |
| | | | | (SEQ ID NO: 135) |
| NM_182965.1 | Hs.68061 | SPHK1 | Sphingosine kinase 1 | 5′-GGG CAA GGC CUU GCA GCU C-3′ |
| | | | | (SEQ ID NO: 136) |
| NM_003329.2 | Hs.435136 | TXN | Thioredoxin | 5′-AUGACUGUCAGGAUGUUGC-3′ |
| | | | | (SEQ ID NO: 137) |
| NM_003329.2 | Hs.435136 | TXN | Thioredoxin | 5′-GCAACAUCCUGACAGUCAU-3′ |
| | | | | (SEQ ID NO: 138) |
| NM_203500.1 | Hs.465870 | KEAP1 | Kelch-like ECH-associated | 5′-UGAACGGUGCUGUCAUGUA-3′ |
| | | | protein 1 | (SEQ ID NO: 139) |
| NM_005239.4 | Hs.517296 | ETS2 | V-ets erythroblastosis virus | 5′-GCAGAGGUUCGGCAUGAAU-3′ |
| | | | E26 oncogene homolog 2 (avian) | (SEQ ID NO: 140) |
| NM_002067.1 | Hs.515056 | GNA11 | Guanine nucleotide binding | 5′-AAGATGTTCGTGGACCTGAAC-3′ |
| | | | protein (G protein), alpha 11 | (SEQ ID NO: 141) |
| | | | (Gq class) | |
| NM_004827.1 | Hs.480218 | ABCG2 | ATP-binding cassette, sub- | 5′-AAGATGATTGTTCGTCCCTGCTATAG |
| | | | family G (WHITE), member 2 | TGAGTCGTATTA-3′ |
| | | | | (SEQ ID NO: 142) |
| NM_000610.3 | Hs.502328 | CD44 | CD44 antigen (homing function | 5′-GAACGAAUCCUGAAGACAUCU-3′ |
| | | | and Indian blood group system) | (SEQ ID NO: 143) |
| NM_003489.1 | Hs.155017 | NRIP1 | Nuclear receptor interacting | 5′-GAAGGAAGCUUUGCUAGCU-3′ |
| | | | protein 1 | (SEQ ID NO: 144) |
| NM_004995.2 | Hs.2399 | MMP14 | atrix metalloproteinase 14 | 5′-AAGCCTGGCTACAGCAATATGCCTGT |
| | | | | CTC-3′ |
| | | | | (SEQ ID NO: 145) |
| NM_022045.2 | Hs.546363 | MTBP | Mdm2, transformed 3T3 cell | 5′-GGCUCAUUUGCACUCAAUU-3′ |
| | | | double minute 2, p53 binding | (SEQ ID NO: 146) |
| | | | protein (mouse) binding | |
| | | | protein, 104 kDa | |
| NM_002392.2 | Hs.369849 | MDM2 | Mdm2, transformed 3T3 cell | 5′-GCCACAAAUCUGAUAGUAU-3′ |
| | | | double minute 2, p53 binding | (SEQ ID NO: 147) |
| | | | protein (mouse) | |
| NM_170707.1 | Hs.491359 | LMNA | Lamin A/C | 5′-CUGGACUUCCAGAAGAACA-3′ |
| | | | | (SEQ ID NO: 148) |
| NM_004759.3 | Hs.519276 | MAPKAPK2 | Mitogen-activated protein | 5′-UGACCAUCACCGAGUUUAU-3′ |
| | | | kinase-activated protein | (SEQ ID NO: 149) |
| | | | kinase 2 | |
| NM_001948.2 | Hs.527980 | DUT | DUTP pyrophosphatase | 5′-GATTATAGGAAATGTTG-3′ |
| | | | | (SEQ ID NO: 150) |
| NM_016022.1 | Hs.108408 | APH-1A | Likely ortholog of C. elegans | 5′-AAGAAGGCAGATGAGGGGTTA-3′ |
| | | | anterior pharynx defective 1A | (SEQ ID NO: 151) |
| NM_031301.2 | Hs.511703 | PSFL | Anterior pharynx defective | 5′-AACAAAGATGGACCAACACAG-3′ |
| | | | 1B-like | (SEQ ID NO: 152) |
| BC007496.1 | Hs.36915 | SMAD3 | SMAD, mothers against DPP | 5′-GGACGAGGUCUGCGUGAAU-3′ |
| | | | homolog 3 (Drosophila) | (SEQ ID NO: 153) |
| NM_182763.1 | Hs.532826 | MCL1 | Myeloid cell leukemia sequence | 5′-AAGAAACGCGGUAAUCGGACU-3′ |
| | | | 1 (BCL2-related) | (SEQ ID NO: 154) |
| NM_001022.3 | Hs.438429 | RPS19 | Ribosomal protein S19 | 5′-GCACAAAGAGCTTGCTCCCTTCAAGAGA |
| | | | | GAGCAAGCTCTTTGTGC-3′ |
| | | | | (SEQ ID NO: 155) |
| NM_001022.3 | Hs.438429 | RPS19 | Ribosomal protein S19 | 5′-GTCCGGGAAGCTGAAAGTCTTCAAGAGA |
| | | | | GACTTTCAGCTTCCCGGAC-3′ |
| | | | | (SEQ ID NO: 156) |
| NM_001022.3 | Hs.438429 | RPS19 | Ribosomal protein S19 | 5′-GAGATCTGGACAGAATCGCTTCAAGAGA |
| | | | | GCGATTCTGTCCAGATCTC-3′ |
| | | | | (SEQ ID NO: 157) |
| NM_001400.2 | Hs.154210 | EDG1 | Endothelial differentiation, | 5′-GAGAACAGCATTAAACTG-3′ |
| | | | sphingolipid G-protein- | (SEQ ID NO: 158) |
| | | | coupled receptor, 1 | |
| NM_001001938.1 | Hs.546252 | C9orf47 | Chromosome 9 open reading | 5′-GGTCAACATTCTGATGTCT-3′ |
| | | | frame 47 | (SEQ ID NO: 159) |
| NM_021972.2 | Hs.68061 | SPHK1 | Sphingosine kinase 1 | 5′-GGGCAAGGCCTTGCAGCTC-3′ |
| | | | | (SEQ ID NO: 160) |
| NM_016068.1 | Hs.423968 | TTC11 | Tetratricopeptide repeat | 5′-GTACAATGATGACATCCGTAA-3′ |
| | | | domain 11 | (SEQ ID NO: 161) |
| NM_016068.1 | Hs.423968 | TTC11 | Tetratricopeptide repeat | 5′-GTACGTCCGCGGGTTGCTGCA-3′ |
| | | | domain 11 | (SEQ ID NO: 162) |
| NM_53831.2 | Hs.395482 | PTK2 | PTK2 protein tyrosine | 5′-AAGCAUGUGGCCUGCUAUGGA-3′ |
| | | | kinase 2 | (SEQ D NO: 163) |
| NM_003749.2 | Hs.442344 | RS2 | Insulin receptor substrate 2 | 5′-GATCCCGCCTCAACAACAACAACAAC |
| | | | | TTCAAGAGAGTTGTTGTTGTTGTTGAGGT |
| | | | | TTTTTGGAAA-3′ |
| | | | | (SEQ ID NO: 164) |
| NM_000691.3 | Hs.531682 | ALDH3A1 | Aldehyde dehydrogenase 3 | 5′-AAGAAGAGCUUCGAGACUUUC-3′ |
| | | | family, member A1 | (SEQ ID NO: 165) |
| NM_000689.3 | Hs.76392 | ALDH1A1 | Aldehyde dehydrogenase 1 | 5′-AACTGGGAGAGTACGGTTTCC-3′ |
| | | | family, member A1 | (SEQ ID NO: 166) |
| NM_000604.2 | Hs.549034 | FGFR1 | Fibroblast growth factor | 5′-AAGTCGGACGCAACAGAGAAA-3′ |
| | | | receptor 1 (fms-related | (SEQ ID NO: 167) |
| | | | tyrosine kinase 2, Pfeiffer | |
| | | | syndrome) | |
| NM_006006.3 | Hs.171299 | ZBTB16 | Zinc finger and BTB domain | 5′-GGCCAACCAGAUGCGGCUGUU-3′ |
| | | | containing 16 | (SEQ ID NO: 168) |
| NM_006006.3 | Hs.171299 | ZBTB16 | Zinc finger and BTB domain | 5′-GAUGUUUGACAUCCUCUUCUU-3′ |
| | | | containing 16 | (SEQ ID NO: 169) |
| NM_004348.1 | Hs.122116 | RUNX2 | Runt-related transcription | 5′-GGCUGCAAGCAGUAUUUACUU-3′ |
| | | | factor 2 | (SEQ ID NO: 170) |
| NM_004348.1 | Hs.122116 | RUNX2 | Runt-related transcription | 5′-GGACAGAGUCAGAUUACAGUU-3′ |
| | | | factor 2 | (SEQ ID NO: 171) |
| NM_014382.2 | Hs.546361 | ATP2C1 | ATPase, Ca++ transporting, | 5′-AGCCACTGTGGAAGAAGTATATT-3′ |
| | | | type 2C, member 1 | (SEQ ID NO: 172) |
| NM_002083.2 | Hs.2704 | GPX2 | Glutathione peroxidase 2 | 5′-CCCUCUGGUUGGUGAUUCA-3′ |
| | | | (gastrointestinal) | (SEQ ID NO: 173) |
| NM_002083.2 | Hs.2704 | GPX2 | Glutathione peroxidase 2 | 5′-GGAUGAUGGCACCUUCCUA-3′ |
| | | | (gastrointestinal) | (SEQ ID NO: 174) |
| NM_000942.4 | Hs.434937 | PPIB | Peptidylprolyl isomerase B | 5′-AATTGGAGATGAAGATGTAGG-3′ |
| | | | (cyclophilin B) | (SEQ ID NO: 175) |
| NM_003153.3 | Hs.524518 | STAT6 | Signal transducer and | 5′-CAGUUCCGCCACUUGCCAA-3′ |
| | | | activator of transcription | (SEQ ID NO: 176) |
| | | | 6, interleukin-4 induced | |
| NM_003153.3 | Hs.524518 | STAT6 | Signal transducer and | 5′-AGCCUGGUGGACAUUUAUU-3′ |
| | | | activator of transcription 6, | (SEQ ID NO: 177) |
| | | | interleukin-4 induced | |
| NM_003153.3 | Hs.524518 | STAT6 | Signal transducer and | 5′-GAUGUGUGAAACUCUGAAC-3′ |
| | | | activator of transcription 6, | (SEQ ID NO: 178) |
| | | | interieukin-4 induced | |
| NM_003153-3 | Hs.524518 | STAT6 | Signal transducer and | 5′-CAGAUGGGUAAGGAUGGCA-3′ |
| | | | activator of transcription 6, | (SEQ ID NO: 179) |
| | | | interleukin-4 induced | |
| NM_002945.2 | Hs.461925 | RPA1 | Replication protein A1, | 5′-AAGCACUAUCAUUGCGAAUCC-3′ |
| | | | 70 kDa | (SEQ ID NO: 180) |
| NM_003169.2 | Hs.437056 | SUPT5H | Suppressor of Ty 5 homolog | 5′-AACTGGGCGAGTATTACATGA-3′ |
| | | | | (SEQ ID NO: 181) |
| NM_003318.3 | Hs.169840 | TTK | TTK protein kinase | 5′-TGAACAAAGTGAGAGACAT-3′ |
| | | | | (SEQ ID NO: 182) |
| NM_007194.3 | Hs.291363 | CHEK2 | CHK2 checkpoint homolog | 5′-AATGTGTGAATGACAACTACT-3′ |
| | | | (S. pombe) | (SEQ ID NO: 183) |
| NM_002358.2 | Hs.533185 | MAD2L1 | MAD2 mitotic arrest | 5′-AATACGGACTCACCTTGCTTG-3′ |
| | | | deficient-like 1 (yeast) | (SEQ ID NO: 184) |
| NM_001401.3 | Hs.126667 | EDG2 | Endothelial differentiation, | 5′-(CCGCCGCUUCCAUUUUUCCU)-3′ |
| | | | lysophosphatidic acid G- | (SEQ ID NO: 185) |
| | | | protein-coupled receptor, 2 | |
| NM_001401.3 | Hs.126667 | EDG2 | Endothelial differentiation, | 5′-(AGGAAAAAUGGAAGCGGCGGG)-3′ |
| | | | lysophosphatidic acid G- | (SEQ ID NO: 186) |
| | | | protein-coupled receptor, 2 | |
| NM_004448.2 | Hs.446352 | ERBB2 | V-erb-b2 erythroblastic | 5′-CCUGGAACUCACCUACCUG-3′ |
| | | | leukemia viral oncogene | (SEQ ID NO: 187) |
| | | | homolog 2 | |
| NM_004448.2 | Hs.446352 | ERBB2 | V-erb-b2 erythroblastic | 5′-CUACCUUUCUACGGACGUG-3′ |
| | | | leukemia viral oncogene | (SEQ ID NO: 188) |
| | | | homolog 2 | |
| NM_004448.2 | Hs.446352 | ERBB2 | V-erb-b2 erythroblastic | 5′-GAUCCGGAAGUACACGAUG-3′ |
| | | | leukemia viral oncogene | (SEQ ID NO: 189) |
| | | | homolog 2 | |
| NM_014812.1 | Hs.533635 | KAB | KARP-1-binding protein | 5′-GAAGGAAUCCUCCAAGUCA-3′ |
| | | | | (SEQ ID NO: 190) |
| NM_002737.2 | Hs.531704 | PRKCA | Protein kinase C, alpha | 5′-AAGCTCCATGTCACAGTACGA-3′ |
| | | | | (SEQ ID NO: 191) |
| NM_212535.1 | Hs.460355 | PRKCB1 | Protein kinase C, beta 1 | 5′-AAGCGCTGCGTCATGAATGTT-3′ |
| | | | | (SEQ ID NO: 192) |
| NM_138578.1 | Hs.516966 | BCL2L1 | BCL2-like 1 | 5′-CTGCCTAAGGCGGATTTGAAT-3′ |
| | | | | (SEQ ID NO: 193) |
| NM_138578.1 | Hs.516966 | BCL2L1 | BCL2-like 1 | 5′-GGCAGGCGACGAGTTTGAACT-3′ |
| | | | | (SEQ ID NO: 194) |
| NM_138578.1 | Hs.516966 | BCL2L1 | BCL2-like 1 | 5′-GTGCGTGGAAAGCGTAGACAA-3′ |
| | | | | (SEQ ID NO: 195) |
| NM_004050.2 | Hs.410026 | BCL2L2 | BCL2-like 2 | 5′-GGCGGAGTTCACAGCTCTATA-3′ |
| | | | | (SEQ ID NO: 196) |
| NM_004050.2 | Hs.410026 | BCL2L2 | BCL2-like2 | 5′-GTGGGCATAAGTGCTGATCTA-3′ |
| | | | | (SEQ ID NO: 197) |
| NM_004050.2 | Hs.410026 | BCL2L2 | BCL2-like 2 | 5′-CTCGGTCCTGCGATTATTAAT-3′ |
| | | | | (SEQ ID NO: 198) |
| NM_003443.1 | Hs.433764 | ZBTB17 | Zinc finger and BTB domain | 5′-AAGGCCGAGATCAGCAAAGTTCAAGA |
| | | | containing 17 | GACTTTGCTGATCTCGGCCTTTTTTTT-3′ |
| | | | | (SEQ ID NO: 199) |
| NM_003345.3 | Hs.302903 | UBE2I | Ubiquitin-conjugating enzyme | 5′-GGCCAGCCAUCACAAUCAA-3′ |
| | | | E2I (UBC9 homolog, yeast) | (SEQ ID NO: 200) |
| NM_003345.3 | Hs.302903 | UBE2I | Ubiquilin-conjugating enzyme | 5′-GGAACUUCUAAAUGAACCA-3′ |
| | | | E2I (UBC9 homolog, yeast) | (SEQ ID NO: 201) |
| NM_016166.1 | Hs.162458 | PIAS1 | Protein inhibitor of | 5′-GGUCCAGUUAAGGUUUUGU-3′ |
| | | | activated STAT, 1 | (SEQ ID NO: 202) |
| NM_016166.1 | Hs.162458 | PIAS1 | Protein inhibitor of | 5′-GGUUACCUUCCACCUACAA-3′ |
| | | | activated STAT, 1 | (SEQ ID NO: 203) |
| NM_004068.2 | Hs.518460 | AP2M1 | Adaptor-related protein | 5′-AAGUGGAUGCCUUUCGGGUCA-3′ |
| | | | complex 2, mu 1 subunit | (SEQ ID NO: 204) |
| NM_004068.2 | Hs.518460 | AP2M1 | Adaptor-related protein | 5′-AAGGAGAACAGUUCUUGCGGC-3′ |
| | | | complex 2, mu 1 subunit | (SEQ ID NO: 205) |
| NM_004068.2 | Hs.518460 | AP2M1 | Adaptor-related protein | 5′-AAGGUCCAGU-CAUUCCAAAUG-3′ |
| | | | complex 2, mu 1 subunit | (SEQ ID NO: 206) |
| NM_001278.2 | Hs.198998 | CHUK | Conserved helix-loop-helix | 5′-AGGAAGGACCUGUUGACCUU-3′ |
| | | | ibiquitous kinase | (SEQ ID NO: 207) |
| NM_001556.1 | Hs.413513 | IKBKB | Inhibitor of kappa light | 5′-UGGUGAGCUUAAUGAAUGA-3′ |
| | | | polypeptide gene enhancer in | (SEQ ID NO: 208) |
| | | | B-cells, kinase beta | |
| NM_021975.2 | Hs.502875 | RELA | V-rel reticuloendotheliosis | 5′-AGAGGACAUUGAGGUGUAU-3′ |
| | | | viral oncogene homolog A | (SEQ ID NO: 209) |
| NM_000963.1 | Hs.196384 | PTGS2 | Prostaglandin-endoperoxide | 5′-AACTGCTCAACACCGGAATTTTT-3′ |
| | | | synthase 2 | (SEQ ID NO: 210) |
| NM_005427.1 | Hs.192132 | TP73 | Tumor protein p73 | 5′-CCAUCCUGUACAACUUCAUGU G-3′ |
| | | | | (SEQ ID NO: 211) |
| NM_005157.2 | Hs.431048 | ABL1 | V-abl Abelson murine leukemia | 5′-CAAUAAGGAAGAAGCCCUU-3′ |
| | | | viral oncogene homolog 1 | (SEQ ID NO: 212) |
| NM_005157.2 | Hs.431048 | ABL1 | V-abl Abelson murine leukemia | 5′-TTAUUCCUUCUUCGGGAAGUC-3′ |
| | | | viral oncogene homolog 1 | (SEQ ID NO: 213) |
| NM_001168.1 | Hs.514527 | BIRC5 | Baculoviral IAP repeat- | 5′-GGCUGGCUUCAUCCACUGC-3′ |
| | | | containing 5 (survivin) | (SEQ ID NO: 214) |
| NM_002940.l | Hs.12013 | ABCE1 | ATP-binding cassette, | 5′-CGAAGATGTTGACCTGGTC-3′ |
| | | | sub-family E (OABP), member 1 | (SEQ ID NO: 215) |
| NM_002940.1 | Hs.12013 | ABCE1 | ATP-binding cassette, | 5′-AGAGTTGTCCTGTAGTTCG-3′ |
| | | | sub-family E (OABP), member 1 | (SEQ ID NO: 216) |
| NM_004208.2 | Hs.424932 | PDCD8 | Programmed cell death 8 | 5′-GGAAAUAUGGGAAAGAUCC-3′ |
| | | | (apoptosis-inducing factor) | (SEQ ID NO: 217) |
| NM_000115.1 | Hs.82002 | EDNRB | Endothelin receptor type B | 5′-GGAGACUUUCAAAUACAUC-3′ |
| | | | | (SEQ ID NO: 218) |
| NM_001712.2 | Hs.512682 | CEACAM1 | Carcinoembryonic antigen- | 5′-AACCTTCTGGAACCCGCCCAC-3′ |
| | | | related cell adhesion | (SEQ ID NO: 219) |
| | | | molecule 1 | |
| NM_001712.2 | Hs.512682 | CEACAM1 | Carcinoembryonic antigen- | 5′-AATGTTGCAGAGGGGAAGGAG-3′ |
| | | | related cell adhesion | (SEQ ID NO: 220) |
| | | | molecule 1 | |
| NM_033284.1 | Hs.436900 | TBL1Y | Transducin (beta)-like 1Y- | 5′-AAGAGAATGGAGCACATGAAA-3′ |
| | | | linked | (SEQ ID NO: 221) |
| NM_033284.1 | Hs.436900 | TBL1Y | Transducin (beta)-like 1Y- | 5′-AAGATGAGCATAACCAGTGAC-3′ |
| | | | linked | (SEQ ID NO: 222) |
| NM_024665.3 | Hs.438970 | TBL1XR1 | Transducin (beta)-like 1X- | 5′-AAGGCCCTATATTTGCATTAA-3′ |
| | | | linked receptor 1 | (SEQ ID NO: 223) |
| NM_173174.1 | Hs.491322 | PTK2B | PTK2B protein tyrosine kinase | 5′-GTTGGCTGAGTGCTATGGGCTGA-3′ |
| | | | 2 beta | (SEQ ID NO: 224) |
| NM_006311.2 | Hs.462323 | NCOR1 | Nuclear receptor co-repressor | 5′-GGGCTTATGGAGGACCCTATGA-3′ |
| | | | 1 | (SEQ ID NO: 225) |
| NM_002211.2 | Hs.429052 | ITGB1 | Integrin, beta 1 | 5′-GGAACAGCAGAGAAGCTCATTCAAGA |
| | | | | GATGAGCTTCTCTGCTGTTCCTTTTT-3′ |
| | | | | (SEQ ID NO: 226) |
| NM_139176.2 | Hs.351118 | NALP7 | NACHT, leucine rich repeal and | 5′-CACCGAAGCAGCACGACTTCTTCTTC |
| | | | 1 PYD containing 7 | AAGAGAGAAGAAGTCGTGCTGCTTC-3′ |
| | | | | (SEQ ID NO: 227) |
| NM_004422.2 | Hs.118640 | DVL2 | Dishevelled, dsh homolog 2 | 5′-AGGUUCAGCAGCUCCACGGA-3′ |
| | | | (Drosophila) | (SEQ ID NO: 228) |
| NM_001228.2 | Hs.369736 | CASP8 | Caspase 8, apoptosis-related | 5′-GATCCCCCCTCGGGGATACTGTCTGA |
| | | | cysteine protease | TTCAAGAGACAGACAGTATCCCCGAGGTT |
| | | | | TTTGGAAA-3′ |
| | | | | (SEQ ID NO: 229) |
| NM_001769.2 | Hs.114286 | CD9 | CD9 antigen (p24) | 5′-GAGCATCTTCGAGCAAGAA-3′ |
| | | | | (SEQ ID NO: 230) |
| NM_004357.3 | Hs.512857 | CD151 | CD151 antigen | 5′-CATGTGGCACCGTTTGCCT-3′ |
| | | | | (SEQ ID NO: 231) |
| NM_003188.2 | Hs.485968 | MAP3K7 | Mitogen-activated protein | 5′-UGGCUUAUCUUACACUGGA-3′ |
| | | | kinase kinase kinase 7 | (SEQ ID NO: 232) |
| NM_006116.2 | Hs.507681 | MAP3K7IP1 | Mitogen-activated protein | 5′-GGCUCAAGUUCAGGAGUGAGAACAA-3′ |
| | | | kinase kinase kinase 7 | (SEQ ID NO: 233) |
| | | | interacting protein 1 | |
| NM_015093.2 | Hs.269775 | MAP3K7IP2 | Mitogen-activated protein | 5′-GGAACGACUUCAAAGAGAACUUGAG-3′ |
| | | | kinase kinase kinase 7 | (SEQ ID NO: 234) |
| | | | interacting protein 2 | |
| NM_001315.1 | Hs.485233 | MAPK14 | Mitogen-activated protein | 5′-GCAUUACAACCAGACAGUUGAUAUU-3′ |
| | | | kinase 14 | (SEQ ID NO: 235) |
| NM_006502.1 | Hs.439153 | POLH | Polymerase (DNA directed), eta | 5′-GUGGAGCAGCGGCAAAAU-3′ |
| | | | | (SEQ ID NO: 236) |
| NM_006502.1 | Hs.439153 | POLH | Polymerase (DNA directed), eta | 5′-UCCUCAUUUGAGGAAUAAA-3′ |
| | | | | (SEQ ID NO: 237) |
| NM_006502.1 | Hs.439153 | POLH | Polymerase (DNA directed), eta | 5′-GGAAUAAACCUUGUGCAGU-3′ |
| | | | | (SEQ ID NO: 238) |
| NM_006502.1 | Hs.439153 | POLH | Polymerase (DNA directed), eta | 5′-UAAACCUUGUGCAGUUGUA-3′ |
| | | | | (SEQ ID NO: 239) |
| NM_006502.1 | Hs.439153 | POLH | Polymerase (DNA directed), eta | 5′-CCUUGUGCAGUUGUACAGU-3′ |
| | | | | (SEQ ID NO: 240) |
| NM_015321.1 | Hs.371096 | MECT1 | Mucoepidermoid carcinoma | 5′-CCGGCAACCUCGCGGCCAAUU-3′ |
| | | | translocated 1 | (SEQ ID NO: 241) |
| NM_181715.1 | Hs.406392 | TORC2 | Transducer of regulated cAMP | 5′-CGACUACCAUCUGCACUUAUU-3′ |
| | | | response clement-binding | (SEQ ID NO: 242) |
| | | | protein (CREB) 2 | |
| NM_001079.3 | Hs.234569 | ZAP70 | Zeta-chain (TCR) associated | 5′-AACCGGCTCTCCATTGGCATT-3′ |
| | | | protein kinase 70 kDa | (SEQ ID NO: 243) |
| NM_004834.3 | Hs.431550 | MAP4K4 | Mitogen-activated protein | 5′-GTGGTTGGAAATGGCACCTTT-3′ |
| | | | kinase kinase kinase kinase 4 | (SEQ ID NO: 244) |
| NM_006191.1 | Hs.524498 | PA2G4 | Proliferation-associated 2G4, | 5′-AAGCGACCAGGAUUAUAUUCU-3′ |
| | | | 38 kDa | (SEQ ID NO: 245) |
| NM_006191.1 | Hs.524498 | PA2G4 | Proliferation-associated 2G4, | 5′-AAGUGAGGUGGAAAGGCGUUU-3′ |
| | | | 38 kDa | (SEQ ID NO: 246) |
| NM_005940.3 | Hs.143751 | MMP11 | Matrix metalloproteinase 11 | 5′-TCCCATGTCCACTTCGACTATGATGTCA |
| | | | (stromelysin 3) | UGAGCATCATAGTCGAAGTGGACATTT-3′ |
| | | | | (SEQ ID NO: 247) |
| NM_005940.3 | Hs.143751 | MMP11 | Matrix metalloproteinase 11 | 5′-TCCCAGATCTACTTCTTCCGAGGTCAAG |
| | | | (stromelysin 3) | AGCCTCGGAAGAAGTAGATCTTT-3′ |
| | | | | (SEQ ID NO: 248) |
| NM_005940.3 | Hs.143751 | MMP11 | Matrix metalloproteinase 11 | 5′-TCCCAGGATGCTGATGGCTATGCCTTCA |
| | | | (stromelysin 3) | AGAGAGGCATAGCCATCAGCATCCTTT-3′ |
| | | | | (SEQ ID NO: 249) |
| NM_003684.3 | Hs.371594 | MKNK1 | MAP kinase interacting | 5′-AATGCCCATCTCTATAGGTTT-3′ |
| | | | serine/threonine kinase 1 | (SEQ ID NO: 250) |
| NM_003668.2 | Hs.413901 | MAPKAPK5 | Mitogen-activated protein | 5′-GGAUAUGCGAAGAAAGAUC-3′ |
| | | | kinase-1 activated protein | (SEQ ID NO: 251) |
| | | | kinase 5 | |
| NM_004604.3 | Hs.83734 | STX4A | Syntaxin 4A (placental) | 5′-AAGGAGGAAGCTGATGAGAAC-3′ |
| | | | | (SEQ ID NO: 252) |
| NM_004177.3 | Hs.530733 | STX3A | Syntaxin 3A | 5′-AACGTCCGGAACAAACTGAAG-3′ |
| | | | | (SEQ ID NO: 253) |
| NM_001009567.1 | Hs.461247 | MRC1L1 | Mannose receptor, C type 1- | 5′-AAGTGGTACGCAGATTGCACG-3′ |
| | | | like 1 | (SEQ ID NO: 254) |
| NM_002576.3 | Hs.435714 | PAK1 | P21/Cdc42/Rac1-activated | 5′-AAGGAGAAGAAAAAGAAGGAC-3′ |
| | | | kinase 1 | (SEQ ID NO: 255) |
| NM_001664.2 | Hs.247077 | RHOA | Ras homolog gene family, | 5′-GCAGGTAGAGTTGGCTTTG-3′ |
| | | | member A | (SEQ ID NO: 256) |
| NM_175744.3 | Hs.502659 | RHOC | Ras homolog gene family, | 5′-GACTATGATCGACTGCGGC-3′ |
| | | | member C | (SEQ ID NO: 257) |
| NM_080491.1 | Hs.429434 | GAB2 | GRB2-associated binding | 5′-GTGAGAACGATGAGAAATA-3′ |
| | | | protein 2 | (SEQ ID NO: 258) |
| NM_080491.1 | Hs.429434 | GAB 2 | GRB2-associated binding | 5′-GTTGGTGCCTAATCACTTA-3′ |
| | | | protein 2 | (SEQ ID NO: 259) |
| NM_005225.1 | Hs.96055 | E2F1 | E2F transcription factor 1 | 5′-GACGTGTCAGGACCTTCGT-3′ |
| | | | | (SEQ ID NO: 260) |
| NM_005225.1 | Hs.96055 | E2F1 | E2F transcription factor 1 | 5′-CTTAACTGGTGTACATTAA-3′ |
| | | | | (SEQ ID NO: 261) |
| NM_006392.2 | Hs.376064 | NOL5A | Nucleolar protein 5A (56 kDa | 5′-CAAUAUGAUCAUCCAGUCCAUUA-3′ |
| | | | with KKE/D repeat) | (SEQ ID NO: 262) |
| NM_015934 | Hs.471104 | NOP5/NOP58 | Nucleolar protein NOP5/NOP58 | 5′-CAAGCAUGCAGCUUCUACCGUUC-3′ |
| | | | | (SEQ ID NO: 263) |
| NM_001436 | Hs.299002 | FBL | Fibrillarin | 5′-CAGUCGAGUUCUCCCACCGCUCU-3′ |
| | | | | (SEQ ID NO: 264) |
| NM_006666 | Hs.515846 | RUVBL2 | RuvB-like 2 (E. coli) | 5′-GAGACCAUCUACGACCUGGGCAC-3′ |
| | | | | (SEQ ID NO: 265) |
| NM_006666 | Hs.515846 | RUVBL2 | RuvB-like 2 (E. coli) | 5′-GAGAGUGACAUGGCGCCUGUCCU-3′ |
| | | | | (SEQ ID NO: 266) |
| NM_003707.1 | Hs.272822 | RUVBL1 | RuvB-like 1 (E. coli) | 5′-AAGGAACCAAACAGUUGAAACUG-3′ |
| | | | | (SEQ ID NO: 267) |
| NM_003707.1 | Hs.272822 | RUVBL1 | RuvB-like 1 (E. coli) | 5′-GAGUCUUCUAUCGCUCCCAUCGU-3′ |
| | | | | (SEQ ID NO: 268) |
| NM_004741 | Hs.523238 | NOLC1 | Nucleolar and coiled-body | 5′-AAAUDGAGGUGGAUUCACGAGUU-3′ |
| | | | phosphoprotein 1 | (SEQ ID NO: 269) |
| NM_032177 | Hs.546453 | PHAX | RNA U, small nuclear RNA | 5′-UAGUAUCAGCGAGGAACAAAUUA-3′ |
| | | | export adaptor | (SEQ ID NO: 270) |
| | | | (phosphorylation regulated) | |
| NM_032177 | Hs.546453 | PHAX | RNA, small nuclear RNA | 5′-AAGAGUAUAUAGCACAGGAUUUA-3′ |
| | | | export adaptor | (SEQ ID NO: 271) |
| | | | (phosphorylation regulated) | |
| NM_024831 | Hs.335068 | NCOA6IP | Nuclear receptor coactivator | 5′-AAGAUUGCCCUUGCUCGCAAUAA-3′ |
| | | | 6 interacting protein | (SEQ ID NO: 272) |
| NM_024831 | Hs.335068 | NCOA6IP | Nuclear receptor coactivator | 5′-UAUCACCGUAUGAAAUGGAAACU-3′ |
| | | | 6 interacting protein | (SEQ ID NO: 273) |
| NM_022874.1 | Hs.202179 | SMN2 | Survival of motor neuron 1, | 5′-AAGUGGAAUGGGUAACUCUUCUU-3′ |
| | | | telomeric | (SEQ ID NO: 274) |
| NM_012321.2 | Hs.515255 | LSM4 | LSM4 homolog, U6 small nuclear | 5′-AACGGCCGUCCCAAAGCUGGCUG-3′ |
| | | | RNA associated | (SEQ ID NO: 275) |
| NM_016200.2 | Hs.446179 | LSM8 | LSM8 homolog, U6 small nuclear | 5′-AAGAAACAGAUUCUGCGCUUGAU-3′ |
| | | | RNA associated | (SEQ ID NO: 276) |
| NM_003142 | Hs.546301 | SSB | Sjogren syndrome antigen B | 5′-GAAUUAGGUCCACUUCAAUGUCC-3′ |
| | | | (autoantigen La) | (SEQ ID NO: 277) |
| NM_003142 | Hs.546301 | SSB | Sjogren syndrome antigen B | 5′-AAGAUUCUUCCAUUAAAUUGCCU-3′ |
| | | | (autoantigen La) | (SEQ ID NO: 278) |
| NM_001228 | Hs.369736 | CASP8 | Caspase 8, apoptosis-related | 5′-AACTACCAGAAAGGTATACCT-3′ |
| | | | cysteine protease | (SEQ ID NO: 279) |
| NM_003842.3 | Hs.521456 | TNFRSF10B | Tumor necrosis factor receptor | 5′-AAGACCCTTGTGCTCGTTGTC-3′ |
| | | | superfamily, member 10b | (SEQ ID NO: 280) |
| NM_017672.2 | Hs.512894 | TRPM7 | Transient receptor potential | 5′-AAGCAGAGTGACCTGGTAGAT-3′ |
| | | | cation channel, subfamily M, | (SEQ ID NO: 281) |
| | | | member 7 | |
| NM_007294.1 | Hs.194143 | BRCA1 | Breast cancer 1, early onset | S′-UCACAGUGUCCUUUAUGUA-3′ |
| | | | | (SEQ ID NO: 282) |
| NM_033238.1 | Hs.526464 | PML | Promyelocytic leukemia | 5′-AUGGCUUCGACGAGUUCAA-3′ |
| | | | | (SEQ ID NO: 283) |
| NM_000546.2 | Hs.408312 | TP53 | Tumor protein p53 (Li-Fraumeni | 5′-GCAUGAACCGGAGGCCCAU-3′ |
| | | | syndrome) | (SEQ ID NO: 284) |
| NM_002198.1 | Hs.436061 | IRF1 | Interferon regulatory factor | 5′-AGACCAGAGCAGGAACAAGTT-3′ |
| | | | 1 | (SEQ ID NO: 285) |
| NM_024790.3 | Hs.370147 | FLJ22490 | Hypothetical protein FLJ22490 | 5′-GAAGATTTGCGCAGTGGAC-3′ |
| | | | | (SEQ ID NO: 286) |
| NM_000546.2 | Hs.408312 | TP53 | Tumor protein p53 (Li-Fraumeni | 5′-UGGUUCACUGAAGACCCAGUU-3′ |
| | | | syndrome) | (SEQ ID NO: 287) |
| NM_002880.2 | Hs.159130 | RAF1 | V-raf-1 murine leukemia viral | 5′-AUUCCUGCUCAAUGGAUUU-3′ |
| | | | oncogene homolog 1 | (SEQ ID NO: 288) |
| NM_098400.1 | Hs.1565 | NEDD4 | Neural precursor cell | 5′-TAGAGCCTGGCTGGGTTGTTTTG-3′ |
| | | | expressed, developmentally | (SEQ ID NO: 289) |
| | | | down-regulated 4 | |
| NM_015277.2 | Hs.185677 | NEDD4L | Neural precussor cell | 5′-AACCACAACACAAAGTCACAG-3′ |
| | | | expressed, developmentally | (SEQ ID NO: 290) |
| | | | down-regulated 4-like | |
| NM_016931.2 | Hs.371036 | NOX4 | NADPH oxidase 4 | 5′-AAACCGGCAGGAGUUUACCCAG-3′ |
| | | | | (SEQ ID NO: 291) |
| NM_005975.2 | Hs.51133 | PTK6 | PTK6 protein tyrosine kinase 6 | 5′-AAGGUGGCCAUUAAGGUGAUU-3′ |
| | | | | (SEQ ID NO: 292) |
| NM_005531.1 | Hs.380250 | IFI16 | Interferon, gamma-inducible | 5′-UCAGAAGACCACAAUCUAC-3′ |
| | | | protein 16 | (SEQ ID NO: 293) |
| NM_000633.1 | Hs.150749 | BCL2 | B-cell CLL/lymphoma 2 | 5′-GUGAAGUCAACAUGCCUGC-3′ |
| | | | | (SEQ ID NO: 294) |
| NM_182981.1 | Hs.528383 | OKL38 | Pregnancy-induced growth | 5′-CACCCUACACGAAGCCAGA-3′ |
| | | | inhibitor | (SEQ ID NO: 295) |
| NM_002961.2 | Hs.81256 | S100A4 | S100 calcium binding protein | 5′-GGA CAG AUG AAG CUG CUUU-3′ |
| | | | A4 | (SEQ ID NO: 296) |
| NM_014585.3 | Hs.529285 | SLC40A1 | Solute carrier family 40 | 5′-GGTGGACAAGAATGCTAGAC-3′ |
| | | | (iron-regulated transporter), | (SEQ ID NO: 297) |
| | | | member 1 | |
| NM_014585.3 | Hs.529285 | SLC40A1 | Solute carrier family 40 | 5′-GAAGGATTGACCAGTTAACC-3′ |
| | | | (iron-regulated transporter), | (SEQ ID NO: 298) |
| | | | member 1 | |
| NM_014585.3 | Hs.529285 | SLC40A1 | Solute carrier family 40 | 5′-GCTTGAACATGAGCAAGAGC-3′ |
| | | | (iron-regulated transporter), | (SEQ ID NO: 299) |
| | | | member 1 | |
| NM_021127.1 | Hs.96 | PMAIP1 | Phorbol-12-myristate-13- | 5′-AACTTCCGGCAGAAACTTCTG-3′ |
| | | | acetate-induced protein 1 | (SEQ ID NO: 300) |
| NM_002467.2 | Hs.202453 | MYC | V-myc myelocytomatosis viral | 5′-GCCACAGCAUACAUCCUGU-3′ |
| | | | oncogene homolog (avian) | (SEQ ID NO: 301) |
| NM_002187.2 | Hs.674 | IL12B | Interleukin 12B | 5′-CGCACGCUAAUGCUGGCAU-3′ |
| | | | | (SEQ ID NO: 302) |
| NM_019887.3 | Hs.169611 | DIABLO | Diablo homolog (Drosophila) | 5′-AAGCGGUGUUUCUCAGAA-3′ |
| | | | | (SEQ ID NO: 303) |
| NM_017563 | Hs.150725 | IL17RD | Interleukin 17 receptor D | 5′-GUCGGAGGGAAGACAGUGC-3′ |
| | | | | (SEQ ID NO: 304) |
| NM_017563 | Hs.150725 | IL17RD | Interleukin 17 receptor D | 5′-GCAUGUGAUUGCUGACGCC-3′ |
| | | | | (SEQ ID NO: 305) |
| NM_003142.2 | Hs.546301 | SSB | Sjogren syndrome antigen B | 5Õ-AAGGCTTCCCAACTGATGCAA-3Õ |
| | | | (autoantigen La) | (SEQ ID NO: 306) |
| NM_003142.2 | Hs.546301 | SSB | Sjogren syndrome antigen B | 5Õ-AAGCCAAGGAAGCATTGGGTA-3Õ |
| | | | (autoantigen La) | (SEQ ID NO: 307) |
| NM_003142.2 | Hs.546301 | SSB | Sjogren syndrome antigen B | 5Õ-AAGTACTAGAA GGAGAGGTGG-3Õ |
| | | | (autoantigen La) | (Seq ID NO: 308) |
| NM_006101 | Hs.414407 | KNTC2 | Kinetochore associated 2 | 5′-GTTCAAAAGCTGGATGATCTT-3′ |
| | | | | (SEQ ID NO: 309) |
| NM_145697 | Hs.234545 | CDCA1 | Cell division cycle associated | 5′-AAGATACGGTCCAGAAGCTTA-3′ |
| | | | 1 | (SEQ ID NO: 310) |
| NM_003550 | Hs.209128 | MAD1L1 | MAD1 mitotic arrest deficient- | 5′-CCAGCGGCTCAAGGAGGTTTT-3′ |
| | | | like 1 | (SEQ ID NO: 311) |
| NM_002358 | Hs.533185 | MAD2L1 | MAD2 mitotic arrest deficient- | 5′-GAGTCGGGACCACAGTTTATT-3′ |
| | | | like 1 | (SEQ ID NO: 312) |
| NM_004336 | Hs.469649 | BUB1 | BUB1 budding uninhibited by | 5′-TAGGCTAATTGTACTGCTCTT-3′ |
| | | | benzimidazoles 1 homolog | (SEQ ID NO: 313) |
| NM_001211.4 | Hs.36708 | BUB1B | BUB1 budding uninhibited by | 5′-GGAGATCCTCTACAAAGGGTT-3′ |
| | | | benzimidazoles 1 homolog beta | (SEQ ID NO: 314) |
| NM_016343.3 | Hs.497741 | CENPF | Centromere protein F, | 5′-AAGAGATGCTAATAGCAGTTT-3′ |
| | | | 350/400 ka (mitosin) | (SEQ ID NO: 315) |
| NM_001813 | Hs.75573 | CENPE | Centromere protein E, 312 kDa | 5′-ACTCTTACTGCTCTCCAGTTT-3′ |
| | | | | (SEQ ID NO: 316) |
| NM_004217 | Hs.442658 | AURKB | Aurora kinase B | 5′-CGAGACCTATCGCCGCATCGT-3′ |
| | | | | (SEQ ID NO: 317) |
| NM_005030 | Hs.329989 | PLK1 | Polo-like kinase 1 | 5′-GGGCGGCTTTGCCAAGTGCTT-3′ |
| | | | | (SEQ ID NO: 318) |
| NM_004104 | Hs.83190 | FASN | Fatty acid synthase | 5′-CCCUGAGAUCCCAGCGCUG-3′ |
| | | | | (SEQ ID NO: 319) |
| NM_021975.2 | Hs.502875 | RELA | V-rel reticuloendotheliosis | 5′-GATCAATGGCTACACAGGA-3′ |
| | | | viral oncogene homolog A | (SEQ ID NO: 320) |
| NM_033256 | Hs.348037 | PPP1R14A | Protein phosphatase 1, | 5′-ACCUGUCGAGGACUUCAUC-3′ |
| | | | regulatory (inhibitor) | (SEQ ID NO: 321) |
| | | | subunit 14A | |
| NM_177966.3 | Hs.151293 | 2′-PDE | 2′-phosphodiesterase | 5′-GUACAAGGUGGAGCGCAAC-3′ |
| | | | | (SEQ ID NO: 322) |
| NM_015355 | Hs.462732 | SUZ12 | Suppressor of zeste 12 homolog | 5′-CCCGGAAATTTCCCGTCCC-3′ |
| | | | | (SEQ ID NO: 323) |
| NM_015355 | Hs.462732 | SUZ12 | Suppressor of zeste 12 homolog | 5′-GAGATGACCTGCATTGCCC-3′ |
| | | | | (SEQ ID NO: 324) |
| NM_016179.1 | Hs.262960 | TRPC4 | Transient receptor potential | 5′-ACUCUUGGUUCAGAAAGGA-3′ |
| | | | cation channel, subfamily C, | (SEQ ID NO: 325) |
| | | | member 4 | |
| NM_000249 | Hs.195364 | MLH1 | MutL homolog 1, colon cancer, | 5′-GGTTCACTACTAGTAAACT-3′ |
| | | | nonpolyposis type 2 | (SEQ ID NO: 326) |
| NM_000534 | Hs.111749 | PMS1 | PMS1 postmeiotic segregation | 5′-GGAATCTACTCGTTTGTAT-3′ |
| | | | increased 1 | (SEQ ID NO: 327) |
| NM_002198 | Hs.436061 | IRF1 | Interferon regulatory factor 1 | 5′-CCAAGAACCAGAGAAAAGA-3′ |
| | | | | (SEQ ID NO: 328) |
| NM_002199.2 | Hs.374097 | IRF2 | Interferon regulatory factor 2 | 5′-CUCUUUAGAAACUGGGCAA-3′ |
| | | | | (SEQ ID NO: 329) |
| NM_000546.2 | Hs.408312 | TP53 | Tumor protein p53 (Li-Fraumeni | 5′-AAGACTCCAGTGGTAATCTAC-3′ |
| | | | syndrome) | (SEQ ID NO: 330) |
| NM_000051 | Hs.435561 | ATM | Ataxia telangiectasia mutated | 5′-TAGAGCTACAGAACGAAAG-3′ |
| | | | (includes complementation | (SEQ ID NO: 331) |
| | | | groups A, C and D) | |
| NM_000051 | Hs.435561 | ATM | Ataxia telangiectasia mutated | 5′-GAATGTGAACACCACCAAA-3′ |
| | | | (includes complementation | (SEQ ID NO: 332) |
| | | | groups A, C and D) | |
| NM_000051 | Hs.435561 | ATM | Ataxia telangiectasia mutated | 5′-CTACACAAATATTGAGGAT-3′ |
| | | | (includes complementation groups | (SEQ ID NO: 333) |
| | | | A, C and D) | |
| NM_000051 | Hs.435561 | ATM | Ataxia telangiectasia mutated | 5′-CTGTACTTCCATACTTGAT-5′ |
| | | | (includes complementation groups | (SEQ ID NO: 334) |
| | | | A, C and D) | |
| NM_001184 | Hs.271791 | ATR | Ataxia telangiectasia and Rad3 | 5′-AAGCCAAGACAAATTCTGTGT-3′ |
| | | | related | (SEQ ID NO: 335) |
| NM_001184 | Hs.271791 | ATR | Ataxia telangiectasia and Rad3 | 5′-AACCTCCGTGATGTTGCTTGA-3′ |
| | | | related | (SEQ ID NO: 336) |
| NM_001798.2 | Hs.19192 | CDK2 | Cyclin-dependent kinase 2 | 5′-CAAAGCCAGAAACAAGTTG-3′ |
| | | | | (SEQ ID NO: 337) |
| NM_001798.2 | Hs.19192 | CDK2 | Cyclin-dependent kinase 2 | 5′-AAATAAACTCTACCTGGTT-3′ |
| | | | | (SEQ ID NO: 338) |
| NM_001798.2 | Hs.19192 | CDK2 | Cyclin-dependent kinase 2 | 5′-AAACCTCAGAATCTGCTTA-3′ |
| | | | | (SEQ ID NO: 339) |
| NM_001798.2 | Hs.19192 | CDK2 | Cyclin-dependent kinase 2 | 5′-GTTACTTCTATGCCTGATT-3′ |
| | | | | (SEQ ID NO: 340) |
| NM_207003 | Hs.469658 | BCL2L11 | BCL2-like 11 (apoptosis | 5′-GACCGAGAAGGUAGACAAUUG-3′ |
| | | | facilitator) | (SEQ ID NO: 341) |
| NM_000166 | Hs.333303 | GJB1 | Gap junction protein, beta 1, | 5′-AAGAGGCACAAGGTCCACATC-3′ |
| | | | 32 kDa | (SEQ ID NO: 342) |
| NM_000359 | Hs.508950 | TGM1 | Transglutaminase 1 | 5′-AUGCAGCUGGAGAUGGCAC-3′ |
| | | | | (SEQ ID NO: 343) |
| NM_024596 | Hs.550532 | MCPH1 | Microcephaly, primary autosomal | 5′-AGGAAGUUGGAAGGAUCCA-3′ |
| | | | recessive 1 | (SEQ ID NO: 344) |
| NM_024596 | Hs.550532 | MCPH1 | Microcephaly, primary autosomal | 5′-GAACACUUAUCAAGCCUAAUU-3′ |
| | | | recessive 1 | (SEQ ID NO: 345) |
| NM_024596 | Hs.550532 | MCPH1 | Microcephaly, primary autosomal | 5′-GGAGAGAACAAGCAUAUUUUU-3′ |
| | | | recessive 1 | (SEQ ID NO: 346) |
| NM_024596 | Hs.550532 | MCPH1 | Microcephaly, primary autosomal | 5′-UGAUGUACCUAUUCUCUUAUU-3′ |
| | | | recessive 1 | (SEQ ID NO: 347) |
| NM_024596 | Hs.550532 | MCPH1 | Microcephaly, primary autosomal | 5′-GAUAAGAGAUUUCAGAAGAUU-3′ |
| | | | recessive 1 | (SEQ ID NO: 348) |
| NM_024596 | Hs.550532 | MCPH1 | Microcephaly, primary autosomal | 5′-GUCACCACAGCGCAATGGA-3′ |
| | | | recessive 1 | (SEQ ID NO: 349) |
| NM_000245 | Hs.132966 | MET | Met proto-oricogene (hepatocyte | 5′-ACUCUAGAUGCUCAGACUU-3′ |
| | | | growth factor receptor) | (SEQ ID NO: 350) |
| NM_205860.1 | Hs.33446 | NR5A2 | Nuclear receptor subfamily 5, | 5′-AGGATCCATCTTCCTGGTTAC-3′ |
| | | | group A, member 2 | (SEQ ID NO: 351) |
| NM_182763.1 | Hs.532826 | MCL1 | Myeloid cell leukemia sequence | 5′-UAACACCAGTACGGACGGG-3′ |
| | | | 1 (BCL2-related) | (SEQ ID NO: 352) |
| NM_008765 | Hs.444870 | ORC2L | Origin recognition complex, | 5′-UGCUCCUCUCAUGUGGGAU-3′ |
| | | | subunit 2-like | (SHQ ID NO: 353) |
| NM_006190 | Hs.444870 | ORC2L | Origin recognition complex, | 5′-UCAUUGGUCAGUUGUCAUC-3′ |
| | | | subunit 2-like | (SEQ ID NO: 354) |
| NM_181837 | Hs.410228 | ORC3L | Origin recognition complex, | 5′-GAGACUUGGGCGGUCAAAU-3′ |
| | | | subunit 3-like | (SEQ ID NO: 355) |
| NM_002592.2 | Hs.147433 | PCNA | Proliferating cell nuclear | 5′-CGGUGACACUCAGUAUGUC-3′ |
| | | | antigen | (SEQ ID NO: 356) |
| NM_016526 | Hs.414418 | BET1L | Blocked early in transport 1 | 5′-AAGCAUGACCAGCCUGCUUAC-3′ |
| | | | homolog (S. cerevisiae) like | (SEQ ID NO: 357) |
| NM_001569 | Hs.522819 | IRAK1 | Interleukin-1 receptor- | 5′-GGUUGUCCUUGAGUAAUAA-3′ |
| | | | associated kinase 1 | (SEQ ID NO: 358) |
| NM_080649 | Hs.73722 | APEX1 | APEX nuclease (multifunctional | 5′-GUCUGGUACGACUGGAGUACC-3′ |
| | | | DNA repair enzyme) 1 | (SEQ ID NO: 359) |
| NM_002658 | Hs.77274 | PLAU | Plasminogen activator, | 5′-AACAUCACTGCTGCAACTGC-3′ |
| | | | urokinase | (SEQ ID NO: 360) |
| NM_001654 | Hs.446641 | ARAF | V-raf murine sarcoma 3611 viral | 5′-AACAACATCTTCCTACATGAG-3′ |
| | | | oncogene homolog | (SEQ ID NO: 361) |
| NM_004333 | Hs.490366 | BRAF | V-raf murine sarcoma viral | 5′-AAAGAATTGGATCTGGATCAT-3′ |
| | | | oncogene homolog B1 | (SEQ ID NO: 362) |
| NM_002880 | Hs.159130 | RAF1 | V-raf-1 murine leukemia viral | 5′-AAUAGUUCAGCAGUUUGGCUA-3′ |
| | | | oncogene homolog 1 | (SEQ ID NO: 363) |
| NM_014314 | Hs.190622 | DDX58 | DEAD (Asp-Glu-Ala-Asp) box | 5′-GAATTTAAAACCAGAATTATC-3′ |
| | | | polypeptide 58 | (SEQ ID NO: 364) |
| NM_000927.3 | Hs.489033 | ABCB1 | ATP-binding cassette, sub- | 5′-AAGCGAAGCAGTGGTTCAGGT-3′ |
| | | | familyB (MDR/TAP), member 1 | (SEQ ID NO: 365) |
| NM_001753.3 | Hs.74034 | CAV1 | Caveolin 1, caveolae protein, | 5′-AGACGAGCUGAGCGAGAAGCA-3′ |
| | | | 22 kDa | (SEQ ID NO: 366) |
| NM_001753.3 | Hs.74034 | CAV1 | Caveolin 1, caveolae protein, | 5′-CAUCUACAAGCCCAACAAC-3′ |
| | | | 22 kDa | (SEQ ID NO: 367) |
| NM_000389.2 | Hs.370771 | CDKN1A | Cyclin-dependent kinase | 5′-CUUCGACUUUGUCACCGAG-3′ |
| | | | inhibitor 1A(p21,Cip1) | (SEQ ID NO: 368) |
| NM_007294.1 | Hs.194143 | BRCA1 | Breast cancer 1, early onset | 5′-AACCTGTCTCCACAAAGTGTG-3′ |
| | | | | (SEQ ID NO: 369) |
| NM_002105 | Hs.477879 | H2AFX | H2A histone family, member X | 5′-CAA CAA GAA GAC GCG AAU C-3′ |
| | | | | (SEQ ID NO: 370) |
| NM_020382 | Hs.443735 | SET8 | PR/SET domain containing | 5′-AAUCGCCUAGGAAGACUGAUC-3′ |
| | | | protein 8 | (SEQ ID NO: 371) |
| NM_012331 | Hs.490981 | MSRA | Methionine sulfoxide reductase | 5′-CCCCUGUAGCGGCCAAACAUU-3′ |
| | | | A | (SEQ ID NO: 372) |
| NM_012331 | Hs.490981 | MSRA | Methionine sulfoxide reductase | 5′-CAAAGUACAAAGGAAUUUAUU-3′ |
| | | | A | (SEQ ID NO: 373) |
| NM_012331 | Hs.490981 | MSRA | Methionine sulfoxide reductase | 5′-CGGGAGGGACAGACUUUCUUU-3′ |
| | | | A | (SEQ PD NO: 374) |
| NM_014554 | Hs.371957 | SENP1 | SUMO1/sentrin specific | 5′-GTGAACCACAACTCCGTATTC-3′ |
| | | | protease 1 | (SEQ ID NO: 375) |
| NM_002945 | Hs.461925 | RPA1 | Replication protein A1, 70 kDa | 5′-AACUGGUUGACGAAAGUGGUG-3′ |
| | | | | (SEQ ID NO: 376) |
| NM_001184 | Hs.271791 | ATR | Ataxia telangiectasia and Rad3 | 5′-AACCCGCGUUGGCGUGGUUGA-3′ |
| | | | related | (SEQ ID NO: 377) |
| NM_001430.3 | Hs.468410 | EPAS1 | Endothelial PAS domain protein | 5′-ACCAAUCCAGCACCCAUCC-3′ |
| | | | 1 | (SEQ ID NO: 378) |
| NM_001530.2 | Hs.509554 | HIF1A | Hypoxia-inducible factor 1, | 5′-CUGAUGACCAGCAACUUGA-3′ |
| | | | alpha subunit (basic helix- | (SEQ ID NO: 379) |
| | | | loop-helix transcription | |
| | | | factor) | |
| NM_021972 | Hs.68061 | SPHK1 | Sphingosine kinase 1 | 5′-GAGCUGCAAGGCCUUGCCC-3′ |
| | | | | (SEQ ID NO: 380) |
| NM_002502 | Hs.73090 | NFKB2 | Nuclear factor of kappa light | 5′-CTCCTCCATTGTGGAACCCAAGGAGC-3′ |
| | | | polypeptide gene enhancer in | (SEQ ID NO: 381) |
| | | | B-cells 2(p49/p100) | |
| NM_016829 | Hs.380271 | OGG1 | 8-oxoguanine DNA glycosylase | 5′-GUAUGGACACUGACUCAGAUU-3′ |
| | | | | (SEQ ID NO: 382) |
| NM_016829 | Hs.38027l | OGG1 | 8-oxoguanine DNA glycosylase | 5′-GUACUUCCAGCUAGAUGUUUU-3′ |
| | | | | (SEQ ID NO: 383) |
| NM_006142 | Hs.523718 | SFN | Stratifin | 5′-GAGCGAAACCUGCUCUCAG-3′ |
| | | | | (SEQ ID NO: 384) |
| NM_006142 | Hs.523718 | SFN | Stratifin | 5′-GGGUGACUACUACCGCUAC-3′ |
| | | | | (SEQ ID NO: 385) |
| NM_006142 | Hs.523718 | SFN | Stratifin | 5′-AGACAGCACCCUCAUCAUG-3′ |
| | | | | (SEQ ID NO: 386) |
| NM_00615 | Hs.477693 | NCK1 | NCK adaptor protein 1 | 5′-GUCCUGGUGGCGAGUUCGA-3′ |
| | | | | (SEQ ID NO: 387) |
| NM_00615 | Hs.477693 | NCK1 | NCK adaptor protein 1 | 5′-CGUCUCUAUGACCUCAACA-3′ |
| | | | | (SEQ ID NO: 388) |
| NM_002422 | Hs.375129 | MMP3 | Matrix metalloproteinase 3 | 5′-AUGAAGAGUCUUCCAAUCCUU-3′ |
| | | | (stromelysin 1, progelatinase) | (SEQ ID NO: 389) |
| NM_000021.2 | Hs.3260 | PSEN1 | Presenilin 1 (Alzheimer | 5′-AAGGTCCACTTCGTATGCTGG-3′ |
| | | | disease 3) | (SEQ ID NO: 390) |
| NM_015331 | Hs.517249 | NCSTN | Nicastrin | 5′-AAGGGCAAGTITCCCGTGCAG-3′ |
| | | | | (SEQ ID NO: 391) |
| NM_016022 | Hs.108408 | APH-1A | Anterior pharynx defective 1 | 5′-AAGAAGGCAGATGAGGGGTTA-3′ |
| | | | homolog A (C. elegans) | (SEQ ID NO: 392) |
| NM_172341 | Hs.534465 | PEN2 | Presenilin enhancer 2 homolog | 5′-AAUCAAAGGCUAUGUCUGGCG-3′ |
| | | | (C. elegans) | (SEQ ID NO: 393) |
| NM_020673 | Hs.529044 | RAB22A | RAB22A, member RAS oncogene | 5′-AAGGACUACGCCGACUCUAUU-3′ |
| | | | family | (SEQ ID NO: 394) |
| NM_001002814 | Hs.191179 | RAB11FIP1 | RAB11 family interacting | 5′-CGCCT TTCCCAGTCCATGT-3′ |
| | | | protein 1 (class I) | (SEQ ID NO: 395) |
| NM_015470 | Hs.24557 | RAB11FIP5 | RAB11 family interacting | 5′-GAGCTGAGTGCTCAGGCTAAA-3′ |
| | | | protein 5 (class I) | (SEQ ID NO: 396) |
| NM_030791 | Hs.24678 | SGPP1 | Sphingosine-1-phosphate | 5′-AGUGGCCCGUUUCCAGCGG-3′ |
| | | | phosphatase 1 | (SEQ ID NO: 397) |
| NM_005406 | Hs.306307 | ROCK1 | Rho-associated, coiled-coil | 5′-AAGGTGATTGGTAGAGGTGCA-3′ |
| | | | containing protein kinase 1 | (SEQ ID NO: 398) |
| NM_198437 | Hs.250822 | STK6 | Serine/threonine kinase 6 | 5′-AAGCACAAAAGCTrGTCTCCA-3′ |
| | | | | (SEQ ID NO: 399) |
| NM_006272 | Hs.422181 | S100B | S100 calcium binding protein, | 5′-GGAAUUCAUGGCCUUUGUU-3′ |
| | | | beta (neural) | (SEQ ID NO: 400) |
| NM_004219 | Hs.350966 | PTTG1 | Pituitary tumor-transforming 1 | 5′-GAUCUCAAGUUUCAACACC-3′ |
| | | | | (SEQ ID NO: 401) |
| NM_004219 | Hs.350966 | PTTG1 | Pituitary tumor-transforming 1 | 5′-GUCUGUAAAGACCAAGGGA-3′ |
| | | | | (SEQ ID NO: 402) |
| NM_001478.2 | Hs.159481 | GALGT | UDP-N-acetyl-alpha-D- | 5′-GGAGCAAGUAGUGGGGCUG-3′ |
| | | | galactosamine: | (SEQ ID NO: 403) |
| | | | (N-acetylneuraminyl)- | |
| | | | galactosylglucosylceramide | |
| | | | N-acetylgalactosaminyl- | |
| | | | transferase | |
| NM_000657 | Hs.150749 | BCL2 | B-cell CLL/lymphoma 2 | 5′-GUACAUCCAUUAUAAGCUG-3′ |
| | | | | (SEQ ID NO: 404) |
| NM_032984 | Hs.368982 | CASP2 | Caspase 2, apoptosis-related | 5′-AACTTCCAGCTGGCATATAGG-3′ |
| | | | cysteine protease | (SEQ ID NO: 405) |
| NM_001228 | Hs.369736 | CASP8 | Caspase 8, apoptosis-related | 5′-AAGGGUCAUGCUCUAUCAGAU-3′ |
| | | | cysteine protease | (SEQ ID NO: 406) |
| NM_197967 | Hs.474150 | BID | BH3 interacting domain death | 5′-AAGAAGACAUCAUCCGGAAUA-3′ |
| | | | agonist | (SEQ ID NO: 407) |
| NM_001167 | Hs.356076 | BIRC4 | Baculoviral IAP repeat- | 5′-AAGGAGAUACCGUGCGGUGCU-3′ |
| | | | containing 4 | (SEQ ID NO: 408) |
| NM_002483 | Hs.466814 | CEACAM6 | Carcinoembryonic antigen- | 5′-CCGGACAGTTCCATGTATA-3′ |
| | | | related cell adhesion molecule | (SEQ ID NO: 409) |
| | | | 6 | |
| NM_001008490 | Hs.285313 | KLF6 | Kruppel-like factor 6 | 5′-GGAGAAAAGCCUUACAGAU-3′ |
| | | | | (SEQ ID NO: 410) |
| NM_024309 | Hs.368551 | TNIP2 | TNFAIP3 interacting protein 2 | 5′-GUAUUUGGCCGCCGACGCA-3′ |
| | | | | (SEQ ID NO: 411) |
| NM_001621 | Hs.171189 | AHR | Aryl hydrocarbon receptor | 5′-AAGACTGGAGAAAGTGGCATG-3′ |
| | | | | (SEQ ID NO: 412) |
| NM_001005845 | Hs.2442 | ADAM9 | A disintegrin and | 5′-AAUCACUGUGGAGACAUUUGC-3′ |
| | | | metalloproteinase domain 9 | (SEQ ID NO: 413) |
| | | | (meltrin gamma) | |
| NM_001110 | Hs.172028 | ADAM10 | A disintegrin and | 5′-AAUGAAGAGGGACACUUCCCU-3′ |
| | | | metalloproteinase domain 10 | (SEQ ID NO: 414) |
| NM_021641 | Hs.386283 | ADAM12 | A disintegrin and | 5′-AACCUCGCUGCAAAGAAUGUG-3′ |
| | | | metalloproteinase domain 12 | (SEQ ID NO: 415) |
| NM_207196 | Hs.312098 | ADAM15 | A disintegrin and | 5′-AACUCCAUCUGUUCUCCUGAC-3′ |
| | | | metalloproteinase domain 15 | (SEQ ID NO: 416) |
| | | | (metargidin) | |
| NM_021832 | Hs.404914 | ADAM17 | A disintegrin and | 5′-AAAGUUUGCUUGGCACACCUU-3′ |
| | | | metalloproteinase domain 17 | (SEQ ID NO: 417) |
| NM_000927.3 | Hs.489033 | ABCB1 | ATP-binding cassette, sub- | 5′-AAGGCCTAATGCCGAACACA-3′ |
| | | | family B (MDR/TAP), member 1 | (SEQ ID NO: 418) |
| NM_000927.3 | Hs.489033 | ABCB1 | ATP-binding cassette, sub- | 5′-AACTTTGGCTGCCATCATCCA-3′ |
| | | | family B (MDR/TAP), member 1 | (SEQ ID NO: 419) |
| NM_000572 | Hs.193717 | IL10 | Interleukin 10 | 5′-UAAGCUCCAAGAGAAAGGC-3′ |
| | | | | (SEQ ID NO: 420) |
| NM_021975 | Hs.502875 | RELA | B-rel reticuloendotheliosis | 5′-GCCCUAUCCCUUUACGUCA-3′ |
| | | | viraloncogene homolog A | (SEQ ID NO: 421) |
| NM_001331 | Hs.166011 | CTNND1 | Catenin (cadherin-associated | 5′-GTGGACCATGCACTGCATGCCTAT |
| | | | protein), delta 1 | AGTGAGTCGTATTAC-3′ |
| | | | | (SEQ ID NO: 422) |
| NM_00121l | Hs.36708 | BUB1B | BUB1 budding uninhibited by | 5′-AGATCCTGGCTAACTGTTC-3′ |
| | | | benzimidazoles 1 homolog beta | (SEQ ID NO: 423) |
| NM_002358 | Hs.533185 | MAD2L1 | MAD2 mitotic arrest deficient- | 5′-TACGGACTCACCTTGCTTG-3′ |
| | | | like 1 (yeast) | (SEQ ID NO: 424) |
| NM_001530.2 | Hs.509554 | HIF1A | Hypoxia-inducible factor 1, | 5′-CUGGACACAGUGUGUUUGA-3′ |
| | | | alpha subunit | (SEQ ID NO: 425) |
| NM_001530.2 | Hs.509554 | HIF1A | Hypoxia-inducible factor 1, | 5′-CUGAUGACCAGCAACUUGA-3′ |
| | | | alpha subunit | (SEQ ID NO: 426) |
| NM_001430 | Hs.468410 | EPAS1 | Endothelial PAS domain | 5′-GCUCUUCGCCAUGGACACA-3′ |
| | | | protein 1 | (SEQ ID NO: 427) |
| NM_001430 | Hs.468410 | EPAS1 | Endothelial PAS domain | 5′-GCGACAGCUGGAGUAUGAA-3′ |
| | | | protein 1 | (SEQ ID NO: 428) |
| NM_001379 | Hs.202672 | DNMT1 | DNA (cytosine-5-)- | 5′-CCAUGAGCACCGUUCUCC-3′ |
| | | | methyltransferase 1 | (SEQ ID NO: 429) |
| NM_031310 | Hs.107125 | PLVAP | Plasmalemma vesicle associated | 5′-CUUGACCAAGGAGCUCAAC-3′ |
| | | | protein | (SEQ ID NO: 430) |
| NM_031310 | Hs.107125 | PLVAP | Plasmalemma vesicle associated | 5′-GGAGCUCAACUUCACCACC-3′ |
| | | | protein | (SEQ ID NO: 431) |
| NM_016734 | Hs.126365 | PAX5 | Paired box gene 5 (B-cell | 5′-CGGCCACUCGCUUCCGGGC-3′ |
| | | | lineage specific activator) | (SEQ ID NO: 432) |
| NM_016734 | Hs.126365 | PAX5 | Paired box gene 5 (B-cell | 5′-GCUCCGUCGACUGCGCGCC-3′ |
| | | | lineage specific activator) | (SEQ ID NO: 433) |
| NM_006257 | Hs.498570 | PRKCQ | Protein kinase C, theta | 5′-AAACCACCGTGGAGCTCTACT-3′ |
| | | | | (SEQ ID NO: 434) |
| NM_006257 | Hs.498570 | PRKCQ | Protein kinase C, theta | 5′-AAGAGCCCGACCTTCTGTGAA-3′ |
| | | | | (SEQ ID NO: 435) |
| NM_032430 | Hs.182081 | BRSK1 | BR serine/threonine kinase 1 | 5′-GUU CUU CCG CCA GAU UGU G-3′ |
| | | | | (SEQ ID NO: 436) |
| NM_015045 | Hs.203099 | KIAA0261 | KIAA0261 | 5′-CGGACUACCCUUAGCACAAUU-3′ |
| | | | | (SEQ ID NO: 437) |
| NM_015045 | Hs.203099 | KIAA0261 | KIAA0261 | 5′-GAAUAGUCACCAUAUUCACUU-3′ |
| | | | | (SEQ ID NO: 438) |
| NM_005430 | Hs.248164 | WNT1 | Wingless-type MMTV integration | 5′-GGTTCCATCGAATCCTGCA-3′ |
| | | | site family, member 1 | (SEQ ID NO: 439) |
| NM_004421.2 | Hs.74375 | DVL1 | Dishevelled, dsh homolog 1 | 5′-AACAAGATCACCTTCTCCGAG-3′ |
| | | | (Drosophila) | (SEQ ID NO: 440) |
| NM_004422 | Hs.118640 | DVL2 | Dishevelled, dsh homolog 2 | 5′-AACTTTGAGAACATGAGCAAC-3′ |
| | | | (Drosophila) | (SEQ ID NO: 441) |
| NM_139049 | Hs.522924 | MAPK8 | Mitogen-activated protein | 5′-CGTGGATTTATGGTCTGTG-3′ |
| | | | kinase 8 | (SEQ ID NO. 442) |
| NM_003376 | Hs.73793 | VEGF | Vascular endothelial growth | 5′-UGGAUGUCUAUCAGCGCAG-3′ |
| | | | factor | (SEQ ID NO: 443) |
| NM_003376 | Hs.73793 | VEGF | Vascular endothelial growth | 5′-GCUACUGCCAUCCAAUCGA-3′ |
| | | | factor | (SEQ ID NO: 444) |
| NM_003376 | Hs.73793 | VEGF | Vascular endothelial growth | 5′-GGAGUACCCUGAUGAGAUC-3′ |
| | | | factor | (SEQ ID NO: 445) |
| NM_003376 | Hs.73793 | VEGF | Vascular endothelial growth | 5′-CUGAGGAGUCCAACAUCAC-3′ |
| | | | factor | (SEQ ID NO: 446) |
| NM_003376 | Hs.73793 | VEGF | Vascular endothelial growth | 5′-CCAAGGCCAGCACAUAGGA-3′ |
| | | | factor | (SEQ ID NO: 447) |
| NM_005123 | Hs.282735 | NR1H4 | Nuclear receptor subfamily 1, | 5′-GTCGTGACTTGCGACAAG-3′ |
| | | | group H, member 4 | (SEQ ID NO: 448) |
| NM_004999 | Hs.149387 | MYO6 | Myosin VI | 5′-GCUGGCAGUUCAUAGGAAU-3′ |
| | | | | (SEQ ID NO: 449) |
| NM_004999 | Hs.149387 | MYO6 | Myosin VI | 5′-CGUGCUCCAAAGUCUGUUA-3′ |
| | | | | (SEQ ID NO: 450) |
| NM_014865 | Hs.5719 | CNAP1 | Chromosome condensation- | 5′-UCAGUAUGUUGUGCAAGAG-3′ |
| | | | related SMC-associated protein | (SEQ ID NO: 451) |
| | | | 1 | |
| NM_014865 | Hs.5719 | CNAP1 | Chromosome condensation- | 5′-GAAGAUACUCUGGAAUUCC-3′ |
| | | | related SMC-associated protein | (SEQ ID NO: 452) |
| | | | 1 | |
| NM_015261 | Hs.438550 | KIAA0056 | KIAA0056 protein | 5′-CUGGAUUUCACAGAGACUG-3′ |
| | | | | (SEQ ID NO: 453) |
| NM_015261 | Hs.438550 | KIAA0056 | KIAA0056 protein | 5′-GCAGAGAUCAUAGAGACUG-3′ |
| | | | | (SEQ ID NO: 454) |
| NM_015341 | Hs.308045 | BRRN1 | Barren homolog (Drosophila) | 5′-GACUUUCCUCAGAAUGACG-3′ |
| | | | | (SEQ ID NO: 455) |
| NM_015341 | Hs.308045 | BRRN1 | Barren homolog (Drosophila) | 5′-CAUUACUCCACCUGUAUCA-3′ |
| | | | | (SEQ ID NO: 456) |
| NM_014551 | Hs.180903 | 384D8-2 | Hypothetical protein 384D8_6 | 5′-GGAUUUCAGGAUGAACACG-3′ |
| | | | | (SEQ ID NO: 457) |
| NM_014551 | Hs.180903 | 384D8-2 | Hypothetical protein 384D8_6 | 5′-GCUGCAGGACUUCCACCAG-3′ |
| | | | | (SEQ ID NO: 458) |
| NM_006031 | Hs.474069 | PCNT2 | Pericentrin 2 (kendrin) | 5′-AAUUGGAACAGCUGCAGCAGA-3′ |
| | | | | (SEQ ID NO: 459) |
| NM_006031 | Hs.474069 | PCNT2 | Pericentrin 2 (kendrin) | 5′-AAGCUCUGAUUUAUCAAAAGA-3′ |
| | | | | (SEQ ID NO: 460) |
| NM_012179.2 | Hs.5912 | FBXO7 | F-box protein 7 | 5′-CCCACACCAUUCCAUUCUA-3′ |
| | | | | (SEQ ID NO: 461) |
| NM_002467 | Hs.202453 | MYC | V-myc myeclocytomatosis viral | 5′-AAGAUGAGGAAGAAAUCGAUGUU-3′ |
| | | | oncogene homolog (avian) | (SEQ ID NO: 462) |
| NM_002467 | Hs.202453 | MYC | V-myc myelocytomatosis viral | 5′-AAAAGGUCAGAGUCUGGAUCACC-3′ |
| | | | oncogene homolog (avian) | (SEQ ID NO: 463) |
| NM_002467 | Hs.202453 | MYC | V-myc myelocytomatosis viral | 5′-CACGUCUCCACACAUCAGCACAA-3′ |
| | | | oncogene homolog (avian) | (SEQ ID NO: 464) |
| NM_002467 | Hs.202453 | MYC | V-myc myelocytomatosis viral | 5′-AAAUGAGAUAAAGGUGGCUAAUU-3′ |
| | | | oncogene homolog (avian) | (SEQ ID NO: 465) |
| NM_002392 | Hs.369849 | MDM2 | Mdm2, transformed 3T3 cell | 5′-UGGUUGCAUUGUCCAUGGC-3′ |
| | | | double minute 2, p53 binding | (SEQ ID NO: 466) |
| | | | protein | |
| NM_003121 | Hs.437905 | SPIB | Spi-B transcription factor | 5′-GATCGCTGTGTGTCTGTAA-3′ |
| | | | (Spi-1/PU.1 related) | (SEQ ID NO: 467) |
| NM_003120.1 | Hs.502511 | SPI1 | Spleen focus forming virus | 5′-GTCCGTATGTAAATCAGAT-3′ |
| | | | (SFFV) proviral integration | (SEQ ID NO: 468) |
| | | | oncogene spi1 | |
| NM_199002 | Hs.278186 | ARHGEF1 | Rho guanine nucleotide | 5′-CATACCATCTCTACCGACG-3′ |
| | | | exchange factor (GEF) 1 | (SEQ ID NO: 469) |
| NM_014784 | Hs.516954 | ARHGEF11 | Rho guanine nucleotide | 5′-ACTGAAGTCTCGGCCAGCT-3′ |
| | | | exchange factor (GEF) 11 | (SEQ ID NO: 470) |
| NM_015313 | Hs.24598 | ARHGEF12 | Rho guanine nucleotide | 5′-GAAACTCGTCGCATCTTCC-3′ |
| | | | exchange factor (GEF) 12 | (SEQ ID NO: 471) |
| NM_173842 | Hs.81134 | IL1RN | Interleukin 1 receptor | 5′-AUCUGCAGAGGCCUCCGCA-3′ |
| | | | antagonist | (SEQ ID NO: 472) |
| NM_032726 | Hs.549218 | PLCD4 | Phospholipase C, delta 4 | 5′-GAGCAGAACCTTCAGAATA-3′ |
| | | | | (SEQ ID NO: 473) |
| NM_032726 | Hs.549218 | PLCD4 | Phospholipase C, delta 4 | 5′-GAGCAGOGCTTCACCATTG-3′ |
| | | | | (SEQ ID NO: 474) |
| NM_032726 | Hs.549218 | PLCD4 | Phospholipase C, delta 4 | 5′-GGAAGGAGAACTAATTCGTA-3′ |
| | | | | (SEQ ID NO: 475) |
| NM_032726 | Hs.549218 | PLCD4 | Phospholipase C, delta 4 | 5′-GATATCATCTTTCTCTGAA-3′ |
| | | | | (SEQ ID NO: 476) |
| NM_004104 | Hs.83190 | FASN | Fatty acid synthase | 5′-CAACTACGGCTTTGCCAAT-3′ |
| | | | | (SEQ ID NO: 477) |
| NM_004104 | Hs.83190 | FASN | Fatty acid synthase | 5′-GCAACTCACGCTCCGGAAA-3′ |
| | | | | (SEQ ID NO: 478) |
| NM_004104 | Hs.83190 | FASN | Fatty acid synthase | 5′-GCCCTGAGCTGGACTACTT-3′ |
| | | | | (SEQ ID NO: 479) |
| NM_004104 | Hs.83190 | FASN | Fatty acid synthase | 5′-GGTATGCGACGGGAAAGTA-3′ |
| | | | | (SEQ ID NO: 480) |
| NM_002165.2 | Hs.504609 | ID1 | Inhibitor of DNA binding 1, | 5′-AACTCGGAATCCGAAGTTGGA-3′ |
| | | | dominant negative helix-loop- | (SEQ ID NO: 481) |
| | | | helix protein | |
| NM_003200.1 | Hs.371282 | TCF3 | Transcription factor 3 | 5′-AAAGACCTGAGGGACCGGGAG-3′ |
| | | | | (SEQ ID NO: 482) |
| NM_015895 | Hs.234896 | GMNN | Geminin, DNA replication | 5′-GAGAAAATGAGCTGTCCGC-3′ |
| | | | inhibitor | (SEQ ID NO: 483) |
| NM_015895 | Hs.234896 | GMNN | Geminin, DNA replication | 5′-CTGGCAGAAGTAGCAGAAC-3′ |
| | | | inhibitor | (SEQ ID NO: 484) |
| NM_006704 | Hs.281902 | SUGT1 | SGT1, suppressor of G2 allele | 5′-AAGGCUUUGGAACAGAAACCA-3* |
| | | | of SKP1 (S. cerevisiae) | (SEQ ID NO: 485) |
| NM_002358 | Hs.533185 | MAD2L1 | MAD2 mitotic arrest | 5′-AAGAGUCGGGACCACAGUUUA-3′ |
| | | | deficient-like 1 | (SEQ ID NO: 486) |
| NM_006472 | Hs.533977 | TXNIP | Thioredoxin interacting | 5′-ACAGACUUCGGAGUACCUG-3′ |
| | | | protein | (SEQ ID NO: 487) |
| NM_001379 | Hs.202672 | DNMT1 | DNA (cytosine-5-)- | 5′-CGGUGCUCAUGCUUACAAC-3′ |
| | | | methyltransferase 1 | (SEQ ID NO: 488) |
| NM_001379 | Hs.202672 | DNMT1 | DNA (cytosine-5-)- | 5′-CGAGUUGCUAGACCGCUUC-3′ |
| | | | methyltransferase 1 | (SEQ ID NO: 489) |
| NM_006838 | Hs.444986 | METAP2 | Methionyl aminopeptidase 2 | 5′-AAUGCCGGUGACACAACAUGA-3′ |
| | | | | (SEQ ID NO: 490) |

It should be appreciated that the sequences listed in Table 1 are non-limiting. Where the sequence listed in Table 1 is an RNA sequence (or where an RNA molecule is prepared having a sequence of, or complementary to, a sequence listed in Table 1), modifications known in the art to stabilize such oligonucleotides (e.g., to reduce RNAse activity), such as the addition of, e.g., dTdT at the 3′ end of the oligonucleotide, such modifications are not reflected in the sequences listed. However, one of ordinary skill would be able to readily envision such modifications and to make such modifications using only standard laboratory protocols and methodology. It should be appreciated that the sequences listed comprise the target-specific sequences and making any additional modifications at the 3′ or 5′ end are within the capabilities of one of ordinary skill and such modified oligomers (RNA and DNA) are included herein.

Other inhibitor molecules that can be used (e.g., packaged in a VLP) include sense and antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat. Med. 4(8):967-71, 1998). For example, neoplastic reversion was obtained using a ribozyme targeted to an H-Ras mutation in bladder carcinoma cells (Feng et al., Cancer Res. 55(10):2024-8, 1995). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer, Nucleic Acids Res. 24(3):423-9, 1996). Multitarget ribozymes (connected or shotgun) have been suggested as a means of improving efficiency of ribozymes for gene suppression (Ohkawa et al., Nucleic Acids Symp Ser. (29):121-2, 1993).

Antisense nucleic acids include modified or unmodified RNA, DNA, or mixed polymer nucleic acids, and primarily function by specifically binding to matching sequences resulting in modulation of peptide synthesis (Wu-Pong, November 1994, BioPharm, 20-33). Antisense nucleic acid binds to target RNA by Watson Crick base-pairing and blocks gene expression by preventing ribosomal translation of the bound sequences either by steric blocking or by activating RNase H enzyme. Antisense molecules may also alter protein synthesis by interfering with RNA processing or transport from the nucleus into the cytoplasm (Mukhopadhyay & Roth, 1996, Crit. Rev. in Oncogenesis 7, 151-190).

As used herein, the term “antisense nucleic acid” describes a nucleic acid that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript. Those skilled in the art will recognize that the exact length of the antisense oligonucleotide and its degree of complementarity with its target will depend upon the specific target selected, including the sequence of the target and the particular bases which comprise that sequence.

Triple helix approaches have also been investigated for sequence-specific gene suppression. Triple helix forming oligonucleotides have been found in some cases to bind in a sequence-specific manner (Postel N., PNAS U.S.A. 88(18):8227-31, 1991; Duval-Valentin et al., PNAS U.S.A. 89(2):504-8, 1992; Hardenbol and Van Dyke PNAS U.S.A. 93(7):2811-6, 1996; Porumb et al., Cancer Res. 56(3):515-22, 1996). Similarly, peptide nucleic acids have been shown to inhibit gene expression (Hanvey et al., Antisense Res. Dev. 1(4):307-17, 1991; Knudsen and Nielson, Nucleic Acids Res. 24(3):494-500, 1996; Taylor et al., Arch. Surg. 132(11):1177-83, 1997). Minor-groove binding polyamides can bind in a sequence-specific manner to DNA targets and hence may represent useful small molecules for future suppression at the DNA level (Trauger et al., Chem. Biol. 3(5):369-77, 1996). In addition, suppression has been obtained by interference at the protein level using dominant negative mutant peptides and antibodies (Herskowitz, Nature 329(6136):219-22, 1987; Rimsky et al., Nature 341(6241):453-6, 1989; Wright et al., PNAS U.S.A. 86(9):3199-203, 1989). In some cases suppression strategies have led to a reduction in RNA levels without a concomitant reduction in proteins, whereas in others, reductions in RNA have been mirrored by reductions in protein.

In some embodiments, VLPs of the invention may be used to package and/or deliver small activating RNAs (saRNAs) and/or snRNA U1 (uRNAs).

It should be appreciated that any of the therapeutic nucleic acids (e.g., siRNA, antisense, etc., nucleic acids) described herein may be prepared (e.g., synthesized or isolated, etc.) and loaded directly into a VLP. However, in some embodiments, a vector nucleic acid (e.g., a linear or circular vector, or plasmid, etc.) encoding one or more of the therapeutic nucleic acids (e.g., operatively connected to a suitable promoter, e.g., a tissue-specific or non-specific promoter) may be loaded into the VLP. As a result, the therapeutic nucleic acid may be expressed at the site of delivery (e.g., when released from the VLP). In some embodiments, a VLP may include both therapeutic nucleic acid(s) and vector nucleic acid(s) encoding therapeutic nucleic acid(s).

Aspects of the invention relate to using VLPs to package and deliver any one or more of the different types of RNA or other nucleic acid molecules described herein. It should be appreciated that in some embodiments any of the different types and/or examples of RNA or other nucleic acid described herein may be modified as described herein to include a sequence motif that binds to an RNA binding amino acid sequence that may be incorporated into a modified coat protein of a VLP as described herein (e.g., in the amino terminal region). One or more such RNA motifs (e.g., translational operators) may be added at the 5′ end, 3′ end, and/or in the middle of any type and/or example of RNA described herein.

Aspects of the invention relate to methods for packaging heterologous agents described herein into VLPs. In some embodiments, methods for packaging may be based on properties of wild-type viral particles and may be adapted for modified viral particles or VLPs.

An electrostatic model for understanding the reversible gating in CCMV has been developed (See Speir, J. A., et al. supra 1995; Zlotnick, A., R. et al., 2000 Virology 277:450-456). The wild type CCMV capsid remains closed below about pH 6.5. Increasing the pH, for example, above pH 6.5, in the absence of Ca²⁺, results in an 10% expansion (swelling) in the overall dimensions of the virion. Swelling results in the creation of sixty 20 Å holes which provide access between the interior and exterior of the virion and the possibility to load of the therapeutic agent. By subsequently lowering the pH to, for example, about pH 5.0 the structural transition of the CCMV capsid from the swollen form to the non-swollen form occurs, thus trapping material, such as a therapeutic agent, within the viral capsid.

In some embodiments, loading of the therapeutic agents into the VLP occurs at about pH 6.0, pH 6.5, pH 7.0, pH 7.5, pH 8.0, pH 8.5, pH 9.0, pH 9.5, pH 10.0, or pH 10.5.

In some embodiments, trapping of the therapeutic agents in the VLP occurs at about pH 6.5, pH 6.0, pH 5.5, pH 5.0, pH 4.5, pH 4.0, pH 3.5, pH 3.0, pH 2.5, or pH 2.0.

In some embodiments, loading and/or trapping may be performed in the absence of Ca²⁺. In some embodiments, loading and/or trapping may be performed in the presence of a chelating agent (e.g., EDTA or other chelating agent). In some embodiments, VLPs are provided that comprise amino acid substitutions in the N-terminal 1-26 amino acids of the coat protein to change the net electrostatic charge of the N-terminal 1-26 amino acids to allow optimal (desired) electrostatic interaction with a therapeutic substance based on the pH used for the swelling of the VLP, the pKₐ of the therapeutic agent and the desired loading density of the agent in the VLP. Additionally, conditions in the target cell during delivery of the therapeutic agent may be taken into account and amino acid substitutions in the N-terminal 1-26 amino acids of the coat protein to change the net electrostatic charge of the N-terminal 1-26 amino acids may be made to generate electrostatic interaction between the inside of the VLP and the therapeutic substance to allow optimal (desired) release of the therapeutic agent at the target site, based on the prevalent pH at the target site, such as e.g., a pH in the physiological range. In some embodiments, a VLP may modified to promote release at a physiological pH within the serum of a subject. However, in some embodiments, a VLP may be modified to reduce or prevent release within the serum of a subject. In some embodiments, a VLP may be modified to promote release at about the pH within a cell or cellular compartment (e.g., endosome and/or lysosome).

In some embodiments, VLPs are provided comprising an N-terminal deletion of the coat protein. For example, a deletion of 34 amino acids at its N terminus (mutant NA34) assembles into T=1 (60 subunits) and T=2 (120 subunits), as well as the wild-type T=3 (180 subunits) particles. These VLPs have outer diameters of 18, 24 and 28 nm, and inner diameters of about 14, 20 and 24 nm respectively.

In certain embodiments, VLPs are provided that may vary in size, e.g., between 10 and 50 nm, or between 15 and 30 nm, or between 18 and 28 nm, particularly VLPs that have an outer diameter of 18 nm, or 24 nm or 28 nm. VLPs of different sizes may be used to control the number of therapeutic molecules that can be loaded into the VLP and the number of therapeutic molecules that can be delivered by the VLP to the target cell or tissue. VLPs of different sizes may also be used to control the bioavailability of the therapeutic agent to the cell by controlling e.g., the rate of resorption or cellular take-up of the VLP by the cell.

In some embodiments, VLPs from viruses other than CCMV can be generated and loaded with therapeutic agents as described herein. For example, VLPs derived from southern bean mosaic virus (a member of the genus sobemovirus) swell similarly to those of CCMV, when treated with EDTA or other chelating agents under mild alkaline conditions.

In some embodiments, VLPs are provided comprising coat protein comprising one or more amino acid deletions and/or substitutions in amino acids 52-176 of the coat protein comprising the five exterior surface-exposed loops, βB-βC (CAAAEAK (SEQ ID NO: 18), aa59-65), βC-αCD1 (ISLP (SEQ ID NO: 19), aa72-75), βD-βE (LPSVSGT (SEQ ID NO: 20), aa98-104), βF-βG (NSKDVVA (SEQ ID NO: 21), aa129-135), βH-βI (SAALTEGD (SEQ ID NO: 22), aa161-168). These loops are not involved in holding the VLPs together and may therefore be modified.

In some embodiments, the external loops are deleted, partially or fully, singly or in groups, or amino acids comprising the loops are substituted, singly or in groups, to reduce the immunogenicity of the VLP while maintaining the ability of the VLP to self-assemble. By “maintaining the ability to self-assemble” it is meant that the VLP maintains some ability to self-assemble. This ability may be reduced if compared to an unmodified VLP.

In some embodiments, amino acids present in loops exposed on the virus surface can be deleted to reduce the immunogenicity of the VLPs.

In some embodiments, the external loops are deleted, partially or fully, singly or in groups, and replaced or substituted, partially or fully, with polypeptides encoding tag sequences that allow the VLP to be purified or labeled, without destroying the ability of the coat protein to form VLPs. In certain embodiments, VLPs are provided comprising insertion of a protein tag, such as an epitope tag, into either the βF-βG or βC-αCD1 loop, which still permits VLP assembly. The resulting chimeric coat protein is a fusion protein of the viral coat protein and the epitope tag sequence(s).

In some embodiments, such a chimeric molecule comprises a fusion of a viral coat protein polypeptide with a tag polypeptide which provides an epitope to which an anti-tag antibody can selectively bind. Provision of the epitope tag enables the VLP comprising the chimeric polypeptide to be readily purified by affinity purification using an anti-tag antibody or another type of affinity matrix that binds to the epitope tag. In other embodiments, the epitope tag enables the VLP comprising the chimeric polypeptide to be readily detectable by contacting the VLP with a labeled anti-tag antibody, or an anti-tag antibody that provides chemical groups that can be utilized for labeling. In some embodiments, the tag may be inserted without deleting or replacing any viral amino acids (e.g., into an external loop without deleting or replacing an external loop amino acid).

Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; tubulin epitope peptide [Skinner et al., J. Biol. Chem., 266:15163-15166 (1991)]; and the T7 gene 10 protein peptide tag [Lutz-Freyermuth et al., PNAS USA, 87:6393-6397 (1990)]. In some embodiments, the tag may be inserted without deleting or replacing any viral amino acids (e.g., into an external loop without deleting or replacing an external loop amino acid).

In some embodiments, the external loops are deleted, partially or fully, singly or in groups, and replaced or substituted, partially or fully, with polypeptides encoding targeting sequences (peptide or moiety) that allow the VLP to be targeted to specific organs, tissues or cells, without destroying the ability of the coat protein to form VLPs. In certain embodiments, VLPs are provided comprising insertion of a targeting peptide into either the βF-βG or βC-αCD1 loop, which still permits VLP assembly. The resulting chimeric coat protein is a fusion protein of the viral coat protein and the targeting peptide sequence(s). In some embodiments, the targeting polypeptide may be inserted without deleting or replacing any viral amino acids (e.g., into an external loop without deleting or replacing an external loop amino acid).

By “targeting moiety” herein is meant a functional group which serves to target or direct the complex to a particular location, cell type, diseased tissue, or association. In general, the targeting moiety is directed against a target molecule and allows concentration of the VLP loaded with the therapeutic agent in a particular localization within a subject. In some embodiments, wherein the targeting moiety is provided as an amino acid sequence comprising a targeting peptide as part of a fusion protein of the coat protein and the targeting peptide, the targeting peptide may be for example RGD targeting peptides (Arap, W., et al., 1998, Science 279:377-80; Pasqualini, R., E. et al., 1995, J Cell Biol 130:1189-96). In certain embodiments, VLPs comprising targeting peptides comprising RGD motifs enter mammalian cells as intact VLPs via active cellular transport, such as receptor-mediated endocytosis or other vesicular traffic mechanisms. In embodiments wherein the VLPs enter the target cell mediated by receptor mediated endocytosis, the VLPs may enter and leave animal cells via the binding interaction between ligand molecules (e.g., the targeting peptides) expressed and displayed by the VLP and their corresponding receptor molecules on the target cell membrane, which causes the membrane to wrap around and engulf the VLP.

In certain embodiments, the VLP may enter the target cell via receptor-mediated endocytosis as a consequence of interaction of the cellular receptor with a targeting peptide displayed on the surface of the VLP. In certain embodiments, the VLP membrane may fuse with the membrane of the endosome membrane, which leads to the release of the therapeutic agent trapped inside the VLP. In certain embodiments, interactions between the interior amino acid residues on the VLP and the therapeutic agent may be optimized for delivery in endosomes that have an acidic pH, about pH 6, ranging from about pH 5.4 to about pH 6.2. As described herein ionic interactions with the therapeutic agent may be altered (e.g., optimized) by altering the net charge of the VLP interior and thereby optimizing delivery at acidic pHs in the endosomes. In certain embodiments, the VLP membrane may fuse with endosomes and/or lysosomes, which have a significantly more acidic pH than endosomes, about pH 4.5. In these embodiments, release of the therapeutic agent may occur in lysosomes. In some embodiments, the release of the therapeutic agent may occur in the cytosol, which is about pH 7.2. As described herein, VLPs can be modified to optimize the release of therapeutic agents into the target cell under various pHs encountered in different structures (e.g., organelles) of the target cell.

In certain embodiments, targeting molecules may be presented on the surface of the VLP either by genetic modifications of the coat protein gene sequence coding for the various loops, creating a (chimeric) fusion protein, or this may be accomplished by chemical means, such as coupling chemistry. In certain embodiments, the CCMV derived VLPs behave similar to those derived from CPMV. For example, the surface exposed loops in CPMV can be changed successfully to display targeting peptides. The RGD-4C peptide, for example, which selectively binds to integrins can be utilized as a cancer cell targeting agent. CPMV particles displaying the ‘FMDV loop’, which contains an RGD motif, in the βB-βC loop of the small protein, is successfully targeted to integrins, the chimeric particles are able to bind to integrin αvβ6 (the natural FMDV receptor), which can be measured, e.g., in an ELISA-based assay. The interaction is highly specific as neither wild-type CPMV nor a mutant in which the RGD motif is mutated to RGE is able to bind (see, for example, N.P. Montague's Ph.D. thesis, “Development of CPMV-based particle technology” University of East Anglia, 2007).

In some embodiments, VLPs are provided comprising fusions of a targeting peptides containing an integrin binding motif. Integrin-binding motifs are characterized in that they comprise the av-containing integrin binding motif, arginine-glycine-aspartic acid (RGD). For example CDCRGDCFC (SEQ ID NO: 507) (Ruoshlati et al. 2003; Aoki et al., “Potential tumor-targeting peptide vector of histidylated oligolysine conjugated to a tumor-homing RGD motif,” Cancer Gene. Ther. 8:783-787 (2001); Pasqualini et al., “αv Integrins as receptors for tumor targeting by circulating ligands,” Nat. Biotechnol., 15(6):542-546 (1997); E. Koivunen et al., “Phage Libraries Displaying Cyclic Peptides with Different Ring Sizes: Ligand Specificities of the RGD-Directed Integrins,” Biotechnology, 13(3):265-170 (1995); Healy et al., “Peptide Ligands for Integrin αvβ3 Selected from Random Phage Display Libraries,” Biochem., 34:3948-3955 (1995)). In certain embodiments, the RGD targeting peptide is NAVPNLRGDLQVLAQKVART (SEQ ID NO: 505).

In some embodiments, VLPs are provided comprising fusions of one or more targeting peptides containing an RGD motif. The RGD-4C peptide, specific to cell surface exposed αvβ3 and αvβ5 integrins, may be expressed as part of the chimeric coat protein described herein, to specifically target melanoma cells that are known to express these integrins. In certain embodiments, increased cellular uptake may also be achieved by these modifications.

In other embodiments, VLPs are provided comprising fusions of one or more targeting peptides containing an RGD motif directed against the αvβ6 integrin receptor and the coat protein, wherein the RGD motif is accessible in the surface-exposed outer shell loops of the VLP. The integrin receptor αvβ6 usually is absent from most healthy adult tissues but is over-expressed in a range of tumours including more than 90% of oral squamous cell carcinomas (OSCC) and approximately 40% of lung and breast carcinomas. As such, αvβ6 is a tumour-specific target. Furthermore, αvβ6 promotes tumorigenesis in vivo through effects on invasion, migration, cell survival and activation of TGFβ. αvβ6 over-expression is associated with poor prognosis in OSCC, colon and breast cancer patients.

Generally, cancers that may be targeted by the VLP comprising one or more targeting peptides provided herein include, but are not limited to melanoma, squamous cell carcinoma, gastric, colon, non small cell lung cancer, or breast cancer.

In other embodiments, the targeting moiety is all or a portion (e.g., a binding portion) of a ligand for a cell surface receptor. Suitable ligands include, but are not limited to, all or a functional portion of the ligands that bind to a cell surface receptor selected from the group consisting of insulin receptor (insulin), insulin-like growth factor receptor (including both IGF-1 and IGF-2), growth hormone receptor, glucose transporters (particularly GLUT 4 receptor), transferrin receptor (transferrin), epidermal growth factor receptor (EGF), low density lipoprotein receptor, high density lipoprotein receptor, leptin receptor, estrogen receptor (estrogen); interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor (VEGF), PDGF receptor (PDGF), transforming growth factor receptor (including TGF- and TGF-), EPO receptor (EPO), TPO receptor (TPO), ciliary neurotrophic factor receptor, prolactin receptor, and T-cell receptors.

In some embodiments, wherein the targeting peptide is basic, acidic amino acids (Asp or Glu) may be further added to the N-terminal and/or C-terminal portion of the targeting peptide to prevent potential problems of particle aggregation that may occur when expressing very basic targeting peptides. Alternatively, the acidic residues may be expressed in an adjacent loop(s) to provide the necessary charge-neutralization.

In some embodiments, the external loops and/or additional parts of the coat protein may be deleted, partially or fully, singly or in groups, and replaced or substituted, partially or fully, with polypeptides encoding e.g., antibodies or antigen-specific fragments, single chain antibodies, nanobodies and/or camel bodies directed against specific receptor molecules such as e.g., the integrin receptor family, the VEGF receptor family, the FGF receptor Family, the IGF receptor family, the EGF receptor family and the hepatocyte receptor. In other embodiments, antibodies or antigen-specific fragments expressed in the external loops of the VLP are directed against one or more tumor-associated antigens (TAAs) or tumor-specific antigens (TSAs), such as viral tumor antigens, cellular oncogene proteins, and/or tumor-associated differentiation antigens, including, but not limited to, CEA, TAG-72 (Yokata et al., 1992, Cancer Res. 52:3402-3408), CO17-1A (Ragnhammar et al., 1993, Int. J. Cancer 53:751-758); GICA 19-9 (Herlyn et al., 1982, J. Clin. Immunol. 2:135), CTA-1 and LEA, Burkitt's lymphoma antigen-38.13, CD19 (Ghetie et al., 1994, Blood 83:1329-1336), human B-lymphoma antigen-CD₂O (Reffet al., 1994, Blood 83:435-445), CD33 (Sgouros et al., 1993, J. Nucl. Med. 34:422-430), melanoma specific antigens such as ganglioside GD2 (Saleh et al., 1993, J. Immunol., 151, 3390-3398), ganglioside GD3 (shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250), tumor-specific transplantation type of cell-surface antigen (TSTA) such as virally-induced tumor antigens including T-antigen DNA tumor viruses and Envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chatterjee et al., 1988, J. of Immunospecifically. 141:1398-1403), neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57) such as I antigen found in fetal erythrocytes, primary endoderm, I antigen found in adult erythrocytes, preimplantation embryos, I(Ma) found in gastric adenocarcinomas, M18, M39 found in breast epithelium, SSEA-1 found in myeloid cells, VEP8, VEP9, Myl, VIM-D5, D₁₅₆₋₂₂ found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T_{5A7} found in myeloid cells, R₂₄ found in melanoma, 4.2, G_{D3}, D1.1, OFA-1, G_{M2}, OFA-2, G_{D2}, and M1:22:25:8 found in embryonal carcinoma cells, and SSEA-3 and SSEA-4 found in 4 to 8-cell stage embryos T cell receptor derived peptides from Cutaneous T cell Lymphoma (Edelson, 1998, The Cancer Journal 4:62), IL-1, IL-2, IL-3, IL-4, IL-5, IL-6, IL-7, IL-8, IL-9, IL-10, IL-11, IL-12, IFN-α, IFN-β, IFN-β 17 mutants, IFN-65, CD2, CD3, CD4, CD5, CD8, CD11a, CD11b, CD11c, CD16, CD18, CD21, CD28, CD32, CD34, CD35, CD40, CD44, CD45, CD54, CD56, OX40L, 4-1BBL, K₂, K1, Pβ, Oα, Mα, Mβ2, Mβ1, Hepsin, Pim-1, LMP1, TAP2, LMP7, TAP1, TRP, Oβ, IAβ, IAα, IEβ, IEβ2, IEα, CYP21, C4B, CYP21P, C4A, Bf, C2, HSP, G7a/b, TNF-α, TNF-β, D, L, Qa, T1a, COL11A2, DPβ2, DPα2, DPβ1, DPα1, DNα, DMα, DMβ, LMP2, TAPi1, LMP7, DOβ, DQβ2, DQα2, DQβ3, DQβ1, DQα1, DRβ, DRα, G250, HSP-70, HLA-B, HLA-C, HLA-X, HLA-E, HLA-J, HLA-A, HLA-H, HLA-G, HLA-F, nerve growth factor, somatotropin, somatomedins, parathormone, FSH, LH, EGF, TSH, THS-releasing factor, HGH, GRHR, PDGF, IGF-I, IGF-II, TGF-β, GM-CSF, M-CSF, G-CSF1, erythropoietin, β-HCG, 4-N-acetylgalactosaminyltransferase, GM2, GD2, GD3, JADE, MART, BAGE, GAGE, MAGE-1, MAGE-2, MAGE-3, XAGE, MUC-1, MUC-2, MUC-3, MUC-4, MUC-18, ICAM-1, C-CAM, V-CAM, ELAM, NM23, EGFR, E-cadherin, N-CAM, LFA-3 (CD58), EpCAM, B7.1, CEA, DCC, PSA, Her2-neu, UTAA, melanoma antigen p 75, K19, HKer 8, pMel 17, TP10, tyrosinase related proteins 1 and 2, p97, p53, RB, APC, DCC, NF-1, NF-2, WT-1, MEN-I, MEN-II, BRCA1, VHL, FCC and MCC, ras, myc, neu, raf, erb, src, fms, jun, trk, ret, gsp, hst, bcl and abl, Clq, Clr, Cls, C4, C2, Factor D, Factor B, properdin, C3, C5, C6, C7, C8, C9, C1Inh, Factor H, C4b-binding protein, DAF, membrane cofactor protein, anaphylatoxin inactivator S protein, HRF, MIRL, CR1, CR2, CR3, CR4, C3a/C4a receptor, C5a receptor, Epstein-Barr Virus antigens (EBNA), BZLF-1, BXLF-1, and Nuclear Matrix Proteins, modified TAAs or TSAs, splice variants of TAAs or TSAs, functional epitopes, epitope agonists, and degenerate variations thereof.

In some embodiments, the external loops and/or additional parts of the coat protein may be deleted, partially or fully, singly or in groups, and replaced or substituted, partially or fully, with polypeptides encoding e.g., targeting peptides derived from virus that have been identified to be responsible for viral-mediated cell entry are provided. For example peptides derived from the Hepatitis B surface antigen which identifies hepatocytes may be used for the treatment of liver disease and hepatocellular carcinoma. Peptides derived from the Human Papilloma Virus which identifies cervical epithelial cells may be used for the treatment of cervical cancer and cervical dysplasia. Peptides derived from the Epstein Barr Virus which identifies lymphocytes may be used for the treatment of lymphoma.

In certain embodiments, the human complement receptor 2 (CR2) binding domain of glycoprotein gp350/220 of the Epstein-Barr virus is a virus-derived targeting peptide that may be used for targeting as described herein.

In certain embodiments, the carboxyl terminus of the HPV L1 protein is a virus-derived targeting peptide that may be used for targeting as described herein.

In certain embodiments, the pre-S2 region of HBV is a virus-derived targeting peptide that may be used for targeting as described herein.

In certain embodiments, the HCV envelope glycoproteins (HCVpp) E1 and E2, specifically amino acids 412-447 within E2 are used for targeting as described herein.

In some embodiments, peptides are used to enhance oral delivery. In certain embodiments, the target tissue is follicle associated epithelium (FAE) overlying Peyer's patches which contains M-cells that have an increased capacity for uptake of particulate antigens. In certain embodiments, an integrin-adherent peptide motif, RGD, can be utilized to achieve selective and improved transport of VLPs into human Peyer's patches to improve oral delivery.

In certain embodiments, mosaic VLP are provided that comprise two or more different wild-type or genetically modified CCMV coat proteins. For example, mosaic VLP may be produced comprising wild-type CCMV coat protein and a modified CCMV coat protein comprising one or more targeting peptides in the one or more surface-exposed loops of the coat protein, e.g., to produce delivery vehicle specific for certain tissues or cells in vivo. In another example, mosaic VLP may be produced comprising wild-type CCMV coat protein, an N-terminal deletion mutant (e.g., amino acids 8-26) of the CCMV coat protein and a modified CCMV coat protein comprising one or more targeting peptides in the one or more surface-exposed loops of the coat protein. In another example, mosaic VLP may be produced comprising wild-type CCMV coat protein, an N-terminal deletion mutant (e.g., amino acids 8-26) of the CCMV coat protein further comprising one or more targeting peptides in the one or more surface-exposed loops of the coat protein.

The ratio between the two or more different types of subunits may be varied at will. In certain embodiments, mosaic VLP may be produced (reassembled) that contain just one subunit comprising a targeting peptide, e.g., to allow specific targeting of the VLP in vivo while all other subunits are wild-type. In certain embodiments, mosaic VLP may be produced that contain just one subunit comprising a targeting peptide while all other subunits are N-terminal deletion mutants (e.g., amino acids 8-26). In certain embodiments, mosaic VLP may be produced that contain just one subunit comprising a targeting peptide while all other subunits comprise N-terminal amino acid substitutions that alter the charge of the interior of the VLP. It will be appreciated that other ratios (e.g., of subunits comprising targeting peptides, N-terminal deletions, N-terminal substitutions, etc. and/or wild-type subunits) are also possible and the invention is not limited in this regard. In some embodiments, the ratio may be 50%:50%, in other embodiments, the ratio may be, for example, 1%:99%, 5%:95%, 10%:90%, 20%:80%, 30%:70%, or 40%:60%. It should further be appreciated that two or more, three or more different subunits may be assembled into VLPs, e.g., subunits that are wild-type, subunits that are N-terminal deletions and subunits that comprise one or more targeting peptides in one or more of the surface exposed loops (or one or more subunits that comprise different targeting peptides, e.g., peptides that target the VLP to specific tissues or cells and peptides that aid cellular uptake). The ratio between the three types of subunits may be varied at will. For example, for three different subunits, the ratios can range from for T=3, 1:1:178, 1:2:177, 1:3:176, 2:2:176, 1:4:175, 2:3:175 subunits etc., until an equal ratio is achieved, e.g., 60:60:60, and all ratios in between. For T=2, the ratio may be any ratio between 1:1:118 and 40:40:40. For T=1, the ratio may be any ratio between 1:1:58 and 20:20:20. In certain embodiments, mosaic VLP are provided comprising CCMV coat proteins comprising a targeting peptide comprising a RGD motif. In certain embodiments, mosaic VLP are provided comprising CCMV coat proteins comprising a targeting peptide comprising the RGD-4C peptide. In certain embodiments, mosaic VLP are provided comprising CCMV coat proteins comprising a targeting peptide comprising a RGD motif specific to cell surface exposed αvβ3, αvβ5, and/or αvβ6 integrins. In certain embodiments, the RGD targeting peptide is NAVPNLRGDLQVLAQKVART (SEQ ID NO: 505).

It should be appreciated that the different ratios of wild type and/or variant VLP described herein in the context of mosaic VLP may refer to either the ratios of VLP coat proteins in an assembled VLP preparation and/or the ratios of VLP coat proteins that are mixed together in a reassembly reaction. Accordingly, when a ratio is described, that ratio may be used as an input ratio for the assembly reaction or that ratio may be the output ratio obtained from an assembly reaction that uses either the same input ratio or a different input ratio that is required to obtain the desired output ratio due to the different properties of the different coat protein variants during the reassembly reaction. Accordingly, in some embodiments, the ratio that is present in a VLP preparation may be the same as that used in the reassembly reaction. However, in some embodiments, the ratios may be different, because one or more variants VLP coat proteins may reassemble less efficiently and/or precipitate and/or otherwise not end up in the final VLP preparation obtained from the reassembly reaction.

In certain embodiments, mosaic VLP are provided comprising (i) CCMV coat proteins comprising the wild-type N-terminal sequence for amino acids 1-26 (SEQ ID NO:1) and optionally comprising one or more targeting moieties and (ii) CCMV coat proteins that comprise N-terminal deletions (e.g., 1-25, 1-26, or 8-26) or modifications (e.g., one or more R to E, R to D, K to E or K to D substitutions within amino acids 8-26) and optionally comprising one or more targeting moieties. In certain embodiments, such mosaic VLP comprise just one subunit that is a CCMV coat protein comprising the wild-type N-terminal sequence for amino acids 1-26 (SEQ ID NO:1), optionally further comprising one or more targeting moieties, while all other subunits of the VLP are CCMV coat proteins that comprise N-terminal deletions or modifications and optionally further comprise one or more targeting moieties. In other embodiments, the mosaic VLP may comprise 2, 3, 4, 5, 10, 15, 20, 25, 30, 35, 40, 45, 50 or more subunits of comprising the wild-type N-terminal sequence for amino acids 1-26 (SEQ ID NO:1), optionally further comprising one or more targeting moieties. In certain embodiments, the ratio of these subunits in such mosaic VLPs is optimized to avoid the encapsidation of heterologous nucleic acid (e.g., RNA of the expression host) during the manufacturing process, while allowing an optimal interaction and stability of the heterologous siRNA that is loaded into the VLP, e.g., for the purpose of in vivo delivery. In certain embodiments, subunit ratios are provided that allow assembly of mosaic VLP comprising targeting peptides (such as integrin-binding peptides) that would not assemble otherwise, or would be very difficult to assemble in reactions with different ratios and/or subunits because of e.g., solubility problems, as described. In certain embodiments, subunit ratios are provided that allow assembly of mosaic VLP comprising targeting peptides (such as integrin-binding peptides) where a majority of subunits (e.g., 60%, 70%, 80%, 90%, 95%, 98%, 99%, 99.9%) is sourced from a preparation of CCMV coat proteins that is made using a nucleic acid encoding for a CCMV coat protein that expresses well in an expression host (e.g., E. coli or P. Pastoris) and the minority (<50%) of subunits is sourced from a preparation of CCMV coat proteins that is made using a nucleic acid encoding for a CCMV coat protein that does not express well in an expression host. It should be appreciated that the expression of the wild-type CCMV coat protein can be easily optimized, whereas expression of certain modified CCMV coat proteins (e.g., modified coat proteins comprising targeting peptides comprising an integrin-binding motif) may be far less optimal. For example expression of such modified coat proteins in a host expression system may be 1×, 2×, 3×, 4×, 5×, 6×, 7×, 8×, 9×, 10×, 20×, 30×, 40×, 50×, 75×, 100×, 500×, 1000×, or less efficient (as measured, e.g., by protein yield) than the expression (production yield) of wild-type CCMV coat protein. As one non-limiting example, precipitation of the modified subunit during coat protein purification from the expression host system may occur and may limit protein yield.

In certain embodiments, one or more surface-exposed amino acids residing in the external loops and/or in other parts of the coat protein may be replaced, substituted or modified, partially or fully, to allow the chemical conjugation of targeting moieties without changing the ability of the protein to self-assemble into VLPs. In certain embodiments, targeting moieties that may be chemically conjugated are antibodies, nanobodies, camel bodies, nucleic acids and aptamers.

In certain embodiments, the total amount of surface-exposed cysteine, and/or lysine, and/or aspartic acid and/or glutamic acid may be increased to provide an increased number of sites allowing chemical conjugation of targeting moieties such as antibodies, nanobodies, camel bodies, nucleic acids and/or aptamers. Surface-exposed thiol (cysteine), amine (lysine) and carboxyl groups (aspartic and glutamic acid) on the VLP may be chemically modified using standard conjugation chemistry, which is well known in the art. As an example, thiols may be conjugated using maleimide derivatives, lysines may be conjugated with N-hydroxysuccinimide (NHS) esters, and carboxylates may be conjugated with N-ethyl-N′-(3-dimethylaminopropyl) carbodiimide (EDC) and/or NHS. The conjugated targeting moieties may be directed against e.g., specific receptor molecules or tumor-associated antigens (TAAs) or tumor-specific antigens (TSAs), as described herein.

In other embodiments, cell surface receptor ligands and hormones, lipids, sugars and dextrans, alcohols, bile acids, fatty acids, amino acids, peptides and nucleic acids may all be attached as described herein to localize or target the VLP to a particular site.

In other embodiments, the targeting moiety is a peptide. Peptides may be attached via chemical linkages to reactive groups on the surface exposed amino acids of the coat protein (Flenniken, M. L., et al. 2005. Chemical Communications: 447-449), (Flenniken, M. L., et al. 2003. Nano Letters 3:1573-1576), (Gillitzer, E., et al. 2002. Chemical Communications: 2390-2391), (Hermanson, G. T. 1996. Academic Press, San Diego), (Wang, Q., et al. 2002. Chemistry & Biology 9:805-811; Wang, Q., et al. 2002. Chemistry & Biology 9:813-819; Wang, Q., et al. 2002. Angewandte Chemie-International Edition 41:459-462)). In some embodiments, peptides are attached to endogenous or engineered reactive functional groups on the surface exposed amino acids of the coat protein.

In some embodiments, one or more surface-exposed amino acids residing in the external loops and/or in other parts of the coat protein may be replaced, substituted or modified, partially or fully, to allow the chemical conjugation of poly(ethylene glycol) PEG or other molecule as described herein.

In certain embodiments, the total amount of surface-exposed lysine, cysteine, histidine, arginine, aspartic acid, glutamic acid, serine, threonine, and/or tyrosine residues may be increased to provide an increased number of sites allowing chemical conjugation PEG or other molecule. PEGylation is routinely achieved by incubation of a reactive derivative of PEG with the target macromolecule, such as a VLP. The covalent attachment of PEG or other molecule to the VLP can “mask” the VLP from the host's immune system and reduces immunogenicity and antigenicity and also improves bioavailability, by influencing the binding affinity of the VLP to the target cell receptors. PEGylation may also alter the absorption and distribution patterns of the VLP.

In some embodiments, mosaic VLPs are provided. In these embodiments, wild-type or genetically modified VLPs, described herein, may be chemically modified by, for example, by PEGylation and then disassembled in vitro. Additionally, chemically unmodified but genetically modified VLPs expressing the inserted (targeting) peptide (or displaying chemically attached targeting moieties), described herein, may be disassembled in vitro and the two types of subunits may be reassembled together, producing mosaic VLPs comprising chemically modified subunits and chemically unmodified subunits expressing the targeting peptide (or displaying chemically attached targeting moieties). The ratio between the two types may be varied at will, e.g., for T=3, 1:179, 2:178, 3:177 subunits, until an equal ratio is achieved, e.g., 90:90, and all ratios in between. For T=2, the ratio may be any ratio between 1:119 and 60:60. For T=1, the ratio may be any ratio between 1:59 and 30:30.

In some embodiments, a VLP comprises just a single subunit expressing the targeting peptide and one or more of the other subunits (e.g., all other subunits) are coated with PEG, polymers like carboxymethyl dextran, a hexahistidine tag, hyaluronic acid, and/or one or more other masking molecules as the invention is not limited in this respect. In these embodiments, the single targeting peptide may be sufficient to allow cell attachment and resorption of the VLP, and PEG, or similar masking molecules, effectively masks the VLP from the immune system of the host even if they are not attached to every subunit. However, it should be appreciated that in some embodiments more than one targeting peptide may be attached to each VLP (e.g., more than one coat protein molecule may have a targeting peptide) as the invention is not limited in this respect.

In some embodiments, to achieve VLPs with just a single subunit expressing the targeting peptide, coat protein subunits are mixed at ratios lower than 1:179, 1:119, or 1:59 (subunit expressing the targeting peptide: PEGylated subunit not expressing the targeting peptide), for example ratios of 0.9, 0.8, 0.7, 0.6, 0.5, 0.4, 0.3, 0.2, 0.1, 0.05, 0.01 subunits expressing the targeting peptide to 180, 120, or 60 PEGylated subunits not expressing the targeting peptide, respectively. In these embodiments, VLPs may form that contain either 1 or 0 subunits expressing the targeting peptide. In these embodiments, the subunit expressing the targeting peptide may also express a tag-sequence or other distinguishable sequence, as described herein, in the same loop or a different loop, or at a different solvent-exposed, exterior site of the coat protein subunit. This tag-sequence may then be used to isolate the VLPs containing 1 subunit expressing the targeting peptide from the VLPs containing no subunits expressing the targeting peptide, using conventional biochemical methods, such as e.g., affinity chromatography.

In certain embodiments, one or more surface-exposed amino acids residing in the external loops and/or in other parts of the coat protein may be replaced, substituted or modified, partially or fully, to allow coating with hyaluronic acid (HA).

HA, a non-sulfated glycosaminoglycan, is a polymer of disaccharides, themselves composed of D-glucuronic acid and D-N-acetylglucosamine, linked together via alternating β-1,4 and β-1,3 glycosidic bonds. The carboxylic acid group of the D-gluconic acid sub-unit can be readily activated to facilitate coupling to amine (lysine) groups on the surface of CCMV or (e.g., using chemical reactions described in Q. Wang, E. Kaltgrad, T. Lin, J. E. Johnson and M. G. Finn. Natural Supramolecular Building Blocks: Wild-Type Cowpea Mosaic Virus. Chemistry & Biology (2002), 9, 805-811; and A. Chatterji, W. F. Ochoa, M. Paine, B. R. Ratna, J. E. Johnson and T. Lin, New Addresses on an Addressable Virus Nanoblock Uniquely Reactive Lys Residues on Cowpea Mosaic Virus. Chemistry & Biology (2004), 11, 855-863), alternatively, modified so as to introduce acetylene or azide functionalities that can then be conjugated to complementary chemically modified sites on the capsid surface via “click”-chemistry (e.g., using chemical reactions described in Q. Wang, T. R. Chan, R. Hilgraf, V. V. Fokin, K. B. Sharpless and M. G. Finn, Bioconjugation by Copper(I)-Catalyzed Azide-Alkyne [3+2] Cycloaddition. J. Amer. Chem. Soc., (2003) 125, 3192-3193; S. S. Gupta, K. S. Raja, E. Kaltgrad, E. Strable and M. G. Finn, Virus-Glycopolymer Conjugates by Copper(I) Catalysis of Atom Transfer Radical Polymerization and Azide-Alkyne Cycloaddition. Chem. Commun., (2005) 4315-4317).

In certain embodiments, the total amount of surface-exposed positively charged residues, such as lysine, and arginine residues may be increased to provide an increased number of positively charged sites allowing coating with hyaluronic acid, a linear negatively charged macromolecule containing a disaccharide repeat unit of N-actylglucosamine and glucuronic acid, present in mammalian extracellular matrix.

In some embodiments, the HA coating solution may be functionalized with targeting moieties, for example, peptides, aptamers, monoclonal antibodies, nanobodies and such, described herein. In other embodiments, the HA coating solution may be functionalized with targeting moieties that are non-protein molecules, such as certain cell receptor ligands, hormones, and lipids, sugars and dextrans, alcohols, bile acids, fatty acids, and nucleic acids.

It should be appreciated that mosaic particles also may be assembled to include one or more proteins that have been modified with hyaluronic acid.

In some embodiments, folic acid may be used as a targeting agent for tumour-specific drug delivery. Folic acid is a well studied targeting agent that binds to a receptor found in abundance on many types of cancer cells. The folic acid receptor is up-regulated in malignancies of the ovary, brain, kidney, breast and lung. In certain embodiments, solvent-exposed amines on the surface of the VLP may be utilized as anchor groups for the selective attachment of folic acid. In certain embodiments, the folic acid may be activated with N-ethyl-N′-(3-dimethylaminopropyl)carbodiimide hydrochloride (EDC) and N-hydroxysuccinimide (NHS) and the activated ester may then be coupled to the amine groups exposed on the VLP surface. In certain embodiments, the integrity of the VLP after folic acid attachment may be monitored by dynamic light scattering, transmission electron microscopy or FT-IR.

It should be appreciated that mosaic particles also may be assembled to include one or more proteins that have been modified with folic acid.

In some embodiments, antibodies may be conjugated using standard coupling methods. For example, antibodies or fragments or derivatives thereof may be conjugated to surface-exposed carboxylates on a VLP. In certain embodiments, the integrity of the antibody-conjugated VLPs may be monitored by HPLC or ELISA.

It should be appreciated that mosaic particles also may be assembled to include one or more proteins that have been modified to include a peptide targeting moiety as described herein.

In certain embodiments, VLPs are provided comprising coat protein comprising amino acid substitutions that allow for control of the opening and closing of the viral perticle. For example, amino acid residues may be substituted for existing amino acid residues to alter the pH sensitivity.

The CCMV capsid undergoes a pH and metal ion dependent reversible structural transition where 60 separate pores in the capsid open or close, exposing the interior of the VLP to the bulk medium. In certain embodiments, VLPs may act as delivery vehicles because of their ability to undergo reversible structural changes allowing for the formation of open pores through which material can pass and can be entrapped. These reversible changes can be controlled by factors such as pH and ionic strength. For example, pH can be used to control the expansion and contraction of the VLP. When the VLP is expanded, e.g., opened, pores are formed allowing for the free exchange of soluble material between the inside and outside of the VLP. When the VLP is contracted, e.g., closed, the pores are closed and any material inside the VLP is trapped within. As this process is freely reversible, the material may be released by placing the VLP under conditions that allow for the expansion of the VLP and the formation of open pores.

In some embodiments, VLPs are provided having a reversible gating mechanism. Such a mechanism may provide controlled encapsulation and release of therapeutic agent.

An electrostatic model for understanding the reversible gating in CCMV has been developed, which allows the design of mutants that can alter the pH dependence of this gating structural transition. (See Speir, J. A., et al. supra 1995; Zlotnick, A., R. et al., 2000 Virology 277:450-456). The wild type cage remains closed below pH 6.5 due to protonation of acidic residues at the pseudo-3-fold axes of the cage. At higher pH, deprotonation results in an electrostatic repulsion at these sites resulting in a swelling transition. Replacement of the acidic residues with neutral or basic residues may have an effect on pH-dependent gating of the protein cage architecture.

In some embodiments, VLPs are modified to provide improved or new chemical switching or gating mechanisms, e.g., chemical switches, that control the reversible swelling of the cages. At pH values lower than about pH 6.5 the virion exists in its compact or closed form. Increasing the pH above, for example, pH 6.5, in the absence of Ca²⁺, results in an 10% expansion (swelling) in the overall dimensions of the virion. Swelling results in the creation of sixty 20A holes which provide access between the interior and exterior of the virion. By lowering the pH to, for example, about pH 5.0 the structural transition of the CCMV virion from the swollen form to the non-swollen form occurs, thus trapping material within the viral cage. In these embodiments, pH acts as a chemical switch for controlling access to and from the central cavity of the CCMV virion.

In some embodiments, loading of the therapeutic agents into the VLP occurs at about pH 6.0, pH 6.5, pH 7.0, pH 7.5, pH 8.0, pH 8.5, pH 9.0, pH 9.5, pH 10.0, or pH 10.5.

In some embodiments, trapping of the therapeutic agents in the VLP occurs at about pH 6.5, pH 6.0, pH 5.5, pH 5.0, pH 4.5, pH 4.0, pH 3.5, pH 3.0, pH 2.5, or pH 2.0.

In some embodiments, loading and/or trapping may be performed in the absence of Ca²⁺. In some embodiments, loading and/or trapping may be performed in the presence of a chelating agent (e.g., EDTA or other chelating agent).

In some embodiments, VLP are modified to provide improved or new chemical switches for the introduction and delivery of therapeutic agents. By “chemical switch” herein is meant a factor present in the microenvironment of the VLP in vitro (e.g., during drug loading) or in vivo (e.g., during drug delivery) that can be used to control the access to and from the VLP's interior. Such a switch can be activated to open and close the pores of the VLP to allow passage of material in and out of the VLP. Examples of chemical switches include pH, ionic strength of the medium, and the like.

In some embodiments, VLPs are genetically modified to be more stable. Native CCMV virions are stable over a broad pH range (pH 2-8) and temperature (−80° C. to 72° C.) (Zhao, X., et al., 1995, Virology, 207:486-494). Empty CCMV virions are stable over this range when assembled from mutants of the coat protein. The salt stable coat protein mutation (K42R) (Fox, J. M., et al., 1996, Virology 222:115-122) and the cysteinyl mutation (R26C) (Fox, J., et al., 1997, Virology 227:229-233.32) both result in empty virions that are stable over this broad pH and temperature range.

Deletions, additions and substitutions of amino acids 52-176 comprising the surface-exposed loops can be generated using standard molecular cloning methods that are well known in the art.

In some embodiments, mosaic VLPs are provided comprising two or more different CCMV coat proteins as described herein. However, it should be appreciated that the invention is not limited to any combinations of two or more different subunits described herein. For example, mosaic VLPs are provided comprising two or more different CCMV coat protein subunits independently selected from the following: i) wild-type; ii) N-terminal deletion mutants (e.g., deletion of amino acids 1-5,1-10, 1-15, 1-20, 1-25, 1-26, 1-30, 1-34, 5-10, 10-15, 15-20, 20-25, 5-25, 10-25, 15-25, 2-25, 2-26, 2-34, 3-26, 4-26, 5-26, 8-26 and any amino acid deletions in between); iii) N-terminal substitution mutants (e.g., substitutions that alter charged amino acids of the wild-type sequence, e.g., one or more of the 9 (e.g., 1 or more, 2 or more, 3, 4, 5, 6, 7, 8, or 9) basic residues (Arg, Lys), e.g., to net negative (Glu or Asp) residues, or any other substitutions that alter the charge based on SEQ ID NO:1); iv) N-terminal substitution mutants e.g., comprising portions of the MS2 coat protein; v) chimeric fusion proteins comprising one or more targeting peptides in one or more of the surface exposed loops (e.g., in amino acids 52-176 of the coat protein comprising the five exterior surface-exposed loops, βB-βC (CAAAEAK (SEQ ID NO: 18), aa59-65), βC-αCD1 (ISLP (SEQ ID NO: 19), aa72-75), βD-βE (LPSVSGT (SEQ ID NO: 20), aa98-104), βF-βG (NSKDVVA (SEQ ID NO: 21), aa129-135), βH-βI (SAALTEGD (SEQ ID NO: 22), aa161-168); vi) wild-type or modified subunits comprising chemically attached targeting moieties (e.g., antibodies or antibody fragments, signaling or targeting peptides, or receptor ligand molecules); and/or vii) wild-type or modified subunits comprising chemically conjugated moieties that e.g., reduce in vivo immunogenicity of the VLP (e.g., PEG) or aid cellular uptake or themselves provide attachment points for further moieties (e.g., HA). It should be appreciated that the two or more different CCMV coat proteins may be different variants within any one of categories ii)-vii). In some embodiments, all of the different CCMV coat proteins in a VLP preparation may be different variants within any one of categories ii)-vii). In some embodiments, a mosaic VLP preparation may include 2, 3, 4, 5, 6, 7, 8, 9, 10, or more different VLP coat proteins.

It should be appreciated that some of the feature described here for individual subunits may also be combined in one subunit, while others are mutually exclusive. One obvious example of a mutually exclusive combination is a subunit that comprises a N-terminal deletion of amino acids 1-26 and N-terminal amino acid substitutions in amino acids 8, 11, 14, and 15. Other examples of a mutually exclusive combination will be apparent to one of ordinary skill.

Combinations that could be combined in one subunit are, for example, features described in i) and vi); i) and vii); ii) and v); ii) and vi); ii) and vii); iii) and v); iii) and vii); iii) and vii); iv) and v); iv) and vi); iv) and vii). Other such combinations of features described herein will be apparent to one of ordinary skill. It should be appreciated that one or more different subunits with combined features may also be used in the assembly of mosaic VLP. The ratio between the two, three, four, five or more different types of subunits may be varied at will, as described herein. In certain embodiments, ratios for two different subunits that might be desirable are, e.g., for T=3, 1:179, 2:178, 3:177 subunits, until an equal ratio is achieved, e.g., 90:90, and all ratios in between. For T=2, the ratio may be any ratio between 1:119 and 60:60. For T=1, the ratio may be any ratio between 1:59 and 30:30. For three different subunits, the ratios can range from for T=3, 1:1:178, 1:2:177, 1:3:176, 2:2:176, 1:4:175, 2:3:175 subunits etc., until an equal ratio is achieved, e.g., 60:60:60, and all ratios in between. For T=2, the ratio may be any ratio between 1:1:118 and 40:40:40. For T=1, the ratio may be any ratio between 1:1:58 and 20:20:20.

It should be appreciated that the resulting ratio after VLP assembly may not necessarily be the ratio in which the different subunits may be added to the reassembly reaction (reassembly mix). It should be appreciated that for various reasons input ratios may not equal output ratios. Input ratio is the ratio of subunits that are added to a reassembly reaction (reassembly mix). Output ratio is the ratio of subunits in the assembled VLP. For example, a subunit having one or more of the feature ii) to vii), may have to be added in 2×, 3×, 4×, 5×, 6×, 7×, 8×, 9×, 10×, 15×, 20×, 30×, 40×, 50×, 100×, 1000× excess (e.g., as compared to wild-type (i)) to contribute equally (e.g., to about 50% for two subunits of about 33% for three subunits) to the resulting VLP. In certain embodiments, VLPs are provided that are useful as delivery agents, such as for the in vivo delivery of one or more therapeutic agents, e.g., one or more anti-cancer agents, to a subject. Anti-cancer agents that may be delivered by the VLPs described herein include, but are not limited to, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; capsitabine; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleulin II (including recombinant interleukin II, or rIL2), interferon alfa-2a; interferon alfa-2b; interferon alfa-nl; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; improsulfan; benzodepa; carboquone; triethylenemelamrine; triethylenephosphoramide; triethylenethiophosphoramide; trimethylolomelainine; chlomaphazine; novembichin; phenesterine; trofosfamide; estermustine; chlorozotocin; gemzar; nimustine; ranimustine; dacarbazine; mannomustine; mitobronitol; aclacinomycins; actinomycin F(1); azaserine; bleomycin; carubicin; carzinophilin; chromomycin; daunorubicin; daunomycin; 6-diazo-5-oxo-1-norleucine; doxorubicin; olivomycin; plicamyciri; porfiromycin; puromycin; tubercidin; zorubicin; denopterin; pteropterin; 6-mercaptopurine; ancitabine; 6-azauridine; carmofur; cytarabine; dideoxyuridine; enocitabine; pulmozyme; aceglatone; aldophosphamide glycoside; bestrabucil; defofamide; demecolcine; elformithine; elliptinium acetate; etoglucid; flutamide; hydroxyurea; lentinan; phenamet; podophyllinic acid; 2-ethylhydrazide; razoxane; spirogermanium; tamoxifen; taxotere; tenuazonic acid; triaziquone; 2,2′,2″-trichlorotriethylamine; urethan; vinblastine; vincristine; vindesine and related agents; 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen; prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol; 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopraminde; MIF inhibitor; mifepristone; miltefosine; nirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody; human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors; microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rheniuim Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division ibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system; erythrocyte gene therapy; velaresol; veramine; verdins; verteporfin; vinorelbine; vinxaltine; vitaxin; vorozole; zanoterone; zeniplatin; zilascorb; or zinostatin stimalamer. In some embodiments, anti-cancer drugs such as 5-fluorouracil, leucovorin, capsitabine, cyclosphosphamide, and gemcitabine may be used alone or in combination with other drugs as described herein (e.g., delived in a VLP, and optionally also as a pharmaceutical preparation in addition to in a VLP).

Gemcitabine, difluorodeoxycytidine, GEMZAR (1-(2-Oxo-4-amino-1,2-dihydropyrimidin-1-yl)-2-deoxy-2,2-difluororibose, or 2′-Deoxy-2′,2′-difluorocytidine, or 2′Deoxy-2′,2′-Difluorocytidine) is a nucleoside analog used as chemotherapy. Gemcitabine is a prodrug that is initially phosphorylated by deoxycytidine kinase to gemcitabine monophosphate, and subsequent phosphorylation steps yield gemcitabine diphosphate and gemcitabine triphosphate (dFdCTP) (Heinemann V et al. Cancer Res 48:4024-4031, 1988). Deamination of dFdCTP to 2′-2′-difluorodeoxyuridine monophosphate (dFdUMP) by the action of dCP-deaminase and subsequently to dFdU represents an important inactivation pathway of gemcitabine.

Gemcitabine is a pyrimidine analog (such as fluorouracil (5-FU)) in which the hydrogens on the 2′ carbons of deoxycytidine are replaced by fluorines. Gemcitabine is converted intracellularly to the active metabolites difluorodeoxycytidine di- and triphosphate (dFdCDP, dFdCTP). dFdCDP disrupts the progression of DNA replication by inhibiting ribonucleotide reductase thereby decreasing the deoxynucleotide pool available for DNA synthesis. Additionally, dFdCTP is incorporated into DNA, resulting in DNA strand termination and apoptosis. Gemcitabine is used to treat various carcinomas: non-small cell lung cancer, pancreatic cancer, bladder cancer and breast cancer. It is being investigated for use in oesophageal cancer, and is used experimentally in lymphomas and various other tumor types. Gemcitabine is the standard treatment in pancreatic cancer care.

Gemcitabine may be given as a drip (infusion) through a fine tube (cannula) inserted into a vein, over a short period of time through a central line, which is inserted under the skin into a vein near the collarbone, or a PICC line inserted into a vein in the crook of your arm. (Martindale: The Complete Drug Reference (35th edition). Eds. Sweetman et al. Pharmaceutical Press, 2007. British National Formulary (54th edition). British Medical Association and Royal Pharmaceutical Society of Great Britain, September 2007).

Examples of other therapeutic agents or imaging agents that may also be delivered in vivo to a subject by the VLPs provided herein, are additional cellular components, genetically engineered or native, recombinant, soluble or any other type of proteins, peptides, cytokines or other signaling molecules, which can have pro- or anti-inflammatory effects, or pro- or anti-apoptotic effects, polysaccharides, glycoproteins, heterogeneous mixtures of macromolecules (e.g., a natural product extract) and hybrid macromolecules (e.g., protein/nucleic acid hybrids, albumin conjugated proteins, drugs, inorganic molecules, organic molecules, or combinations thereof), or other bioactive molecules, such as growth factors, for example members of the transforming growth factor—β (TGF-β) super family, bone morphogenetic proteins (BMPs), fibroblast growth factors, growth hormone, and insulin-like growth factors (IGFs), antibodies, other nucleic acids (e.g., RNA, DNA, PNA, multiplexes of them (e.g., triplex)), preferably siRNA and antisense RNA, and/or cytotoxic drugs. The diagnostic, prophylactic or therapeutic substances used may be sterile. In some embodiments, the substances and/or agents are sterilized prior to loading into a VLP. In some embodiments, the loaded VLP may sterilized. Sterilization may be achieved using any suitable technique including chemical, radiation, and/or filtering provided that the technique does not inactivate or remove the agent or VLP of interest. Accordingly, a filter may be used if it has a cut-off that is larger than the size of the loaded VLP. Radiation may be used provided that the VLP does not contain an active nucleic acid, unless the level of radiation is sufficient to sterilize the preparation without inactivating the nucleic acid to such an extent that the preparation is rendenred ineffective.

A therapeutic substance may also be any of the following agents: adrenergic agent; adrenocortical steroid; adrenocortical suppressant; agents for treating cognition, antiplatelets, aldosterone antagonist; amino acid; anabolic; analeptic; analgesic; anesthetic; anorectic; anti-acne agent; anti-adrenergic; anti-allergic; anti-Alzheimer's, anti-amebic; anti-anemic; anti-anginal; anti-arthritic; anti-asthmatic; anti-atherosclerotic; antibacterial; anticholinergic; anticoagulant; anticonvulsant; antidepressant; antidiabetic; antidiarrheal; antidiuretic; anti-emetic; anti-epileptic; antifibrinolytic; antifungal; antihemorrhagic; antihistamine; antihyperlipidemia; antihypertensive; antihypotensive; anti-infective; anti-inflammatory; antimicrobial; antimigraine; antimitotic; antimycotic, antinauseant, antineoplastic, antineutropenic, antiparasitic; antiproliferative; antipsychotic; antirheumatic; antiseborrheic; antisecretory; antispasmodic; antithrombotic; anti-ulcerative; antiviral; anxiolytics, appetite suppressant; blood glucose regulator; bone resorption inhibitor; bronchodilator; cardiovascular agent; cholinergic; COX1 inhibitors, COX2 inhibitors, direct thrombin inhibitors, depressant; diagnostic aid; diuretic; dopaminergic agent; estrogen receptor agonist; fibrinolytic; fluorescent agent; free oxygen radical scavenger; gastrointestinal motility effector; glucocorticoid; GPIIbIIIa antagonists, hair growth stimulant; hemostatic; histamine H2 receptor antagonists; hormone; human growth hormone, hypocholesterolemic; hypoglycemic; hypolipidemic; hypnotics, hypotensive; imaging agent; immunological agents such as immunizing agents, immunomodulators, immunoregulators, immunostimulants, and immunosuppressants; keratolytic; LHRH agonist; mood regulator; mucolytic; mydriatic; nasal decongestant; neuromuscular blocking agent; neuroprotective; NMDA antagonist; non-hormonal sterol derivative; plasminogen activator; platelet activating factor antagonist; platelet aggregation inhibitor; proton pump inhibitors, psychotropic; radioactive agent; scabicide; sclerosing agent; sedative; sedative-hypnotic; selective adenosine A1 antagonist; serotonin antagonist; serotonin inhibitor; serotonin receptor antagonist; statins, steroid; thyroid hormone; thyroid inhibitor; thyromimetic; tranquilizer; amyotrophic lateral sclerosis agent; cerebral ischemia agent; Paget's disease agent; unstable angina agent; vasoconstrictor; vasodilator; wound healing agent; or xanthine oxidase inhibitor, but it is not so limited.

In certain embodiments, the therapeutic molecule is an anti-cancer drug, an antibiotic, an anti-viral agent, an anti-microbial agent, an anti-inflammatory agent, or an immunostimulatory agent, but is not so limited.

A “diagnostic substance” is any substance that has diagnostic capabilities, for example imaging agents, such as detectable markers, for example heavy metals, Gadolinium, Quantum dots, magnetic particle, radioactive particles, labeled antibodies, luciferase and other chemoluminescent agents. These agents may be substances inside the hollow nanoparticle, and/or on the surface (e.g., outer-surface) of the particle (e.g., membrane). These agents may be used to detect an adverse condition by any medical detection device or method, such as for example Magnetic Resonance Imaging (MRI), Positron Emission Tomography (PET), Computerized Axial Tomography (CAT), X-rays, or other imaging modalities. These applications may provide for immediate monitoring and/or diagnosis of early metastasis. It should be appreciated that these imaging embodiments may be combined with delivery embodiments, for example, to allow for simultaneous treatment and monitoring (e.g., to confirm that treatment is appropriately localized to a target site such as a tumor or other diseased tissue).

In certain embodiments, the therapeutic molecule may be a gene. In certain embodiments, VLP can be used for gene delivery, for example comprising a heterologous nucleic acid molecule that is an expression vector for a gene. Expression vectors useful for gene delivery are known in the art. In some embodiments, the gene to be delivered in vivo by the VLP encodes a cytokine or other signaling molecule. In certain embodiments, the cytokine or other signaling molecule is pro- or anti-inflammatory. In other embodiments, the cytokine or other signaling molecule is pro- or anti-apoptotic. Suitable cytokines or signaling molecules are known in the art.

In some embodiments, CCMV particles may be expressed in plants using the CPMV-HT system for the expression of foreign proteins in plants as described in Sainsbury, F. and Lomonossoff, G. P. Extremely High-Level and Rapid Transient Protein Production in Plants without the Use of Viral Replication. Plant Physiol. 2008 Sep. 5. In these embodiments, the sequence of the CCMV coat protein and its derivatives, as described herein, may be inserted into the CPMV-HT expression cassette in a binary plasmid and the constructs may be agro-infiltrated into N. benthamiana and assembled particles extracted from the leaves.

In some embodiments, a yeast-based heterologous protein expression system (e.g., Pichia pastoris) for the large scale production of wild type and modified CCMV proteins (e.g., protein cages). In some embodiments, an E. coli-based CCMV coat protein expression system can be used (Zhao, X., et al., 1995. Virology 207:486-494). However, other prokaryotic (e.g., bacterial), eukaryotic (e.g., yeast, insect, mammalian, including human) cells may be used.

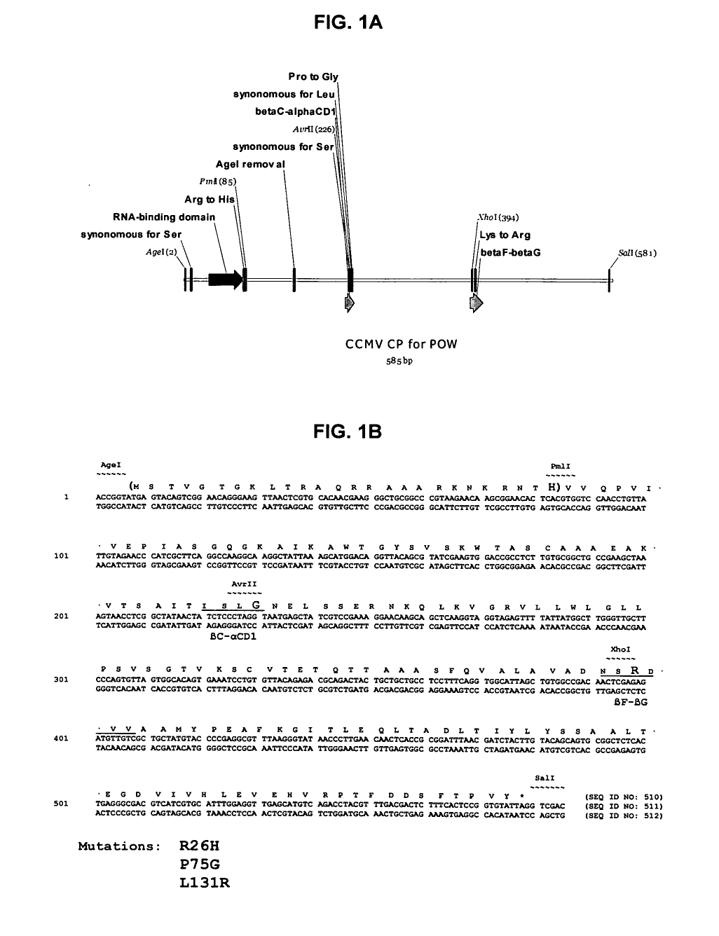

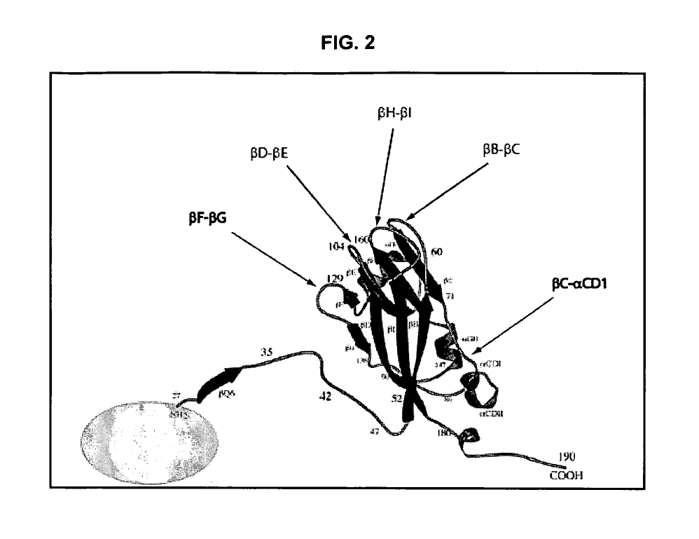

In certain embodiments, synthetic DNAs and polynucleotides encoding the modified coat protein, targeting peptides or portions thereof described herein are provided (see, e.g., FIGS. 1, 3, 6 and 8). In certain embodiments, the coding sequence is codon optimized for expression in a particular host, e.g., yeast, human cells or bacteria.

In certain embodiments, host cells are provided comprising the synthetic DNA or polynucleotides described herein. In certain embodiments, the host cell is a mammalian cell, a bacteria (e.g., E. coli), or a yeast. (e.g., P. pastoris).

The individual coat protein subunits can be produced in heterologous systems such as Escherichia coli or Pichia pastoris, and can be purified by conventional methods.

In some embodiments, CCMV coat protein particles assemble in vitro into empty, RNA-free VLPs, that is they can assemble without the presence of CCMV viral RNA, or any other RNA. In certain embodiments, the therapeutic agent to be loaded is added to the coat proteins prior or during VLP assembly. In certain embodiments, the therapeutic agent to be loaded is added to the coat proteins after they have formed a VLP. The ability to produce coat protein subunits in heterologous systems makes it feasible to scale-up the low-cost production of the agent-loaded VLPs to gram or kilogram quantities, so that any pharmaceutical or therapeutic applications will be economically viable.

In vitro assembly of a VLP can be performed by reassembling disassembled coat proteins that are obtained from any suitable source. In some embodiments, coat proteins (e.g., unassembled coat proteins) may be isolated by disassembling a VLP (e.g., an assembled or partially assembled VLP isolated from a cell culture). A VLP may be disassembled using any suitable method including varying the pH or salt concentration, adding a denaturant, a detergent, a chaotropic agent, a chelating agent, or any combination thereof. In some embodiments, a VLP is disassembled without denaturing the coat proteins. However, in some embodiments, coat proteins may be denatured or partially denatured. In some embodiments, unassembled coat proteins are isolated directly from an expression system that does not promote assembly of the VLP (e.g., an in vitro expression system, or a cell that does not support significant assembly of the VLP). In some embodiments, a modified VLP described herein may not assemble efficiently (e.g., even in a system that promotes or supports assembly of a wild-type CCMV VLP). Accordingly, the modified VLP may be isolated directly in a form that is suitable for reassembly (e.g., without requiring disassembly).

Regardless of the procedure used to obtain one or more coat proteins suitable for reassembly, the coat proteins may be reassembled using any suitable technique. For example, a coat protein in a solution or buffer that promotes disassembly (or that stabilizes unassembled coat proteins) may be reassembled by changing the solution or buffer to one that promotes assembly (e.g., by dilution, dialysis, changing the pH, adding a salt, using a column, or any combination thereof). Accordingly, a mosaic may be made by mixing unassembled proteins of different types. It should be appreciated that in some embodiments an unassembled protein preparation as used herein may nonetheless contain a small percentage of assembled VLPs, but the protein preparation should be sufficiently disassembled and/or the assembled VLPs should be sufficiently unstable to allow reassembly, e.g., to form mosaic VLPs. Techniques for the disassembly and reassembly of CCMV coat proteins are known in the art, see, for example, Lavelle et al., J. Virol. Methods, 2007 December, 146(1-2): 311-6, the disassembly, reassembly and stability techniques of which are incorporated herein by reference.

In some embodiments, synthetic coat protein genes are expressed in E. coli or P. pastoris or other suitable prokaryotic or eukaryotic host cells. In some embodiments, a host cell may include two or more different coat protein genes that express different coat proteins (e.g., wild-type and one or more variants, or two or more variants with no wild-type). Coat protein preparations from such cells may be used directly to form mosaic VLP. It should be appreciated that the relative expression levels of the different coat proteins may be selected and determined using different promoters and/or regulatory sequences.

Synthetic coat protein genes may be produced synthetically by one of the companies specializing in this technology (e.g., GeneArt, Sloning). In these embodiments, gene synthesis is carried out to optimize codon usage for the host system that produced the coat protein and/or to eliminate unwanted restriction sites. In some embodiments, altered forms of the coat protein, such as the NA34 mutant are generated, that give rise to VLPs that have diameters of about 18, 24 and 28 nm. In some embodiments, modifications may be made to produce VLPs having other diameters. VLPs of a particular size may be isolated from other sized VLPs using methods known in the art.

In some embodiments, the synthetic genes may be inserted into pET-based expression plasmids and expressed in E. coli or P. pastoris using procedures which are routinely used in the art. In certain embodiments, the expressed CCMV coat protein may be extracted, purified and assembled into VLPs using routine procedures.

In some embodiments, the synthetic CCMV coat protein genes may be inserted into the pPICZ shuttle vector (InVitrogen, Inc.) and integrated into the P. pastoris genome. Expression of the coat protein may be under the control of a strong methanol inducible promoter (e.g., the AOX1 promoter). In certain embodiments, methanol induction results in the high level expression of the coat protein that self-assembles into empty virus particles within P. pastoris.

In certain embodiments, methods of preparing a mosaic VLP preparation are provided, the method comprising combining at least two different CCMV coat proteins, wherein at least one coat protein is modified as described herein so that a mosaic VLP is generated. In certain embodiments, the methods further comprise combining a therapeutic molecule, a diagnostic molecule, or a heterologous nucleic acid with the at least two different CCMV coat proteins. Combining or loading of the therapeutic molecule, diagnostic molecule, or heterologous nucleic acid may be done e.g., during VLP assembly, as described herein. In certain embodiments, the therapeutic molecule is an anti-cancer drug, an antibiotic, an anti-viral agent, an anti-microbial agent, an anti-inflammatory agent, or an immunostimulatory agent; the diagnostic molecule is an imaging agent; and the heterologous nucleic acid is a heterologous RNA molecule, a microRNA (miRNA), a short interfering RNA (siRNA), a chemically modified short interfering RNA, a double-stranded RNA (dsRNA), a short hairpin RNA (shRNA), RNAu, a circular siRNA, a hybrid DNA-siRNA, a crook siRNA, an antisense RNA molecule, or an expression vector comprising a gene, but is not so limited. In certain embodiments, the anti-cancer drug can be Docetaxel, Paclitaxel, Capecitabine, Doxorubicin, or Rapamycin, but is not so limited.

In some embodiments, VLPs are provided to deliver a therapeutic agent (e.g., Gemcitabine) in vivo to a target cell or tissue in a subject. It should be appreciated that VLPs may protect a therapeutic agent (e.g., Gemcitabine) from modification (e.g., deamination) in the plasma of a subject, thereby enhancing the amount of active drug in the targeted cells.

In some embodiments, VLPs may be delivered orally. In certain embodiments, VLPs can be used for oral delivery of cytotoxic drugs.

In some embodiments, VLPs may be delivered subcutaneously, intravenously, intra-peritoneally, or via any other suitable route.

In certain embodiments, methods of treating a subject having an adverse condition are provided, the methods comprising administering to the subject a VLP preparation described herein or a pharmaceutical composition comprising a VLP preparation and optionally a non-VLP pharmaceutical composition in an amount effective to treat the condition. In certain embodiments, the adverse condition is a tumor, asthma, liver disease, heart disease, and Alzheimer's disease, but is not so limited. In certain embodiments, where the adverse condition is a tumor, the tumor can be a melanoma, squamous cell carcinoma, gastric, colon, non small cell lung cancer, or breast cancer, but is not so limited.

Provided herein, in certain embodiments, are uses of a VLP preparation for preventing or treating an adverse condition. In certain embodiments, use of a VLP preparation for the manufacture of a medicament for preventing or treating an adverse condition are provided. In certain embodiments, the adverse condition is selected from the group consisting of a tumor, asthma, liver disease, heart disease, and Alzheimer's disease, but is not so limited. In certain embodiments, where the adverse condition is a tumor, the tumor can be a melanoma, squamous cell carcinoma, gastric, colon, non small cell lung cancer, or breast cancer, but is not so limited.

The term “effective amount” of a composition refers to the amount necessary or sufficient for a composition alone, or together with further doses, to realize a desired biologic effect. A compound or composition, such as a VLP preparation when “administered in a sufficient amount,” alone or together with further doses or, where indicated, together with additional compound or compositions, realizes a desired biologic effect. The desired response or effect, of course, will depend on the particular condition being treated. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or adverse condition being treated, the size of the subject, or the severity of the disease or adverse condition. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons. One of ordinary skill in the art can empirically determine the effective amount without necessitating undue experimentation.

For any compound described herein the therapeutically effective amount can be initially determined from animal models. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

As used herein, the terms “treat,” “treated,” or “treating” when used with respect to an adverse condition, such as a disorder or disease (e.g., cancer, infection, neurodegenerative disorder, or any other disease or disorder) may refer to prophylaxis, amelioration, prevention and/or cure of the condition. Treatment after a condition (e.g., disease or disorder) has started aims to reduce, ameliorate or altogether eliminate the condition, and/or its associated symptoms, or prevent it from becoming worse. Treatment of subjects before a condition has started (e.g., prophylactic treatment) aims to reduce the risk of developing the condition and/or lessen its severity if the condition does develop. As used herein, the term “prevent” refers to the prophylactic treatment of a subject who is at risk of developing a condition resulting in a decrease in the probability that the subject will develop the disorder, and/or to the inhibition of further development of an already established disorder. Desired outcomes may include a stabilization of the condition, a slowdown in progression of the disease or a full disease-free recovery of the subject.

The VLPs described herein may be administered per se (neat) or in the form of a pharmaceutically acceptable formulation. If the VLPs are administered in pharmaceutically acceptable solutions, they may routinely contain pharmaceutically acceptable concentrations of salt, buffering agents, preservatives, compatible carriers, adjuvants, and optionally other therapeutic ingredients. The solutions used preferably are sterile. The pharmaceutical compositions contain an effective amount of VLPs optionally included in a pharmaceutically-acceptable carrier, and may be sterilized as described herein.

Provided herein are pharmaceutical compositions comprising the VLP preparation described herein optionally further comprising a non-VLP pharmaceutical compound.

Modes of administering the VLPs and therapeutic agents described herein will vary depending upon the specific agents used and the disease being treated, as would either be known to those skilled in the art or can be established by routine experimentation using methods commonly employed in the art. Dependent upon these factors, the agents may be administered orally or parenterally. In some embodiments, oral formulations may include immediate release particle coatings. In some embodiments, oral formlulations may include controlled release particle coatings. In some embodiments, oral formulations may include extended release particle coatings. Parenteral modes of administration include intravenous, intramuscular, subcutaneous, intradermal, intraperitoneal, intralesional, intrapleural, intrathecal, intra-arterial, and into lymphatic vessels or nodes and to bone or bone marrow. The VLPs and therapeutic agents of the invention may also be administered topically or transdermally, buccally or sublingually, or by a nasal, pulmonary, vaginal, or anal route. Accordingly, in some embodiments, compositions of the invention may be provided in the form of tablets, capsules, softgels, liquids, powders, or other forms for oral administration. In some embodiments, compositions of the invention may be provided in the form of liquid or lyophilized preparations for injection. In some embodiments, compositions of the invention may be provided in the form of a metered dose, a dry powder, a nebulized, or a nasal preparation for respiratory delivery. In some embodiments, these or other formulations may be used for ophthalmic, otic, topical, and/or other forms of delivery.

For oral administration, the pharmaceutical compositions can be formulated readily by combining the active compound(s), e.g., the VLPs described herein and optionally additional therapeutic agents, with pharmaceutically acceptable carriers well known in the art. Such carriers enable the VLPs and optionally additional therapeutic agents to be formulated as tablets, pills, dragees, capsules, liquids, gels, hydrogels, pellets, granules, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

The compositions may be administered by inhalation to pulmonary tract, especially the bronchi and more particularly into the alveoli of the deep lung, using standard inhalation devices. The compositions may be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. An inhalation apparatus may be used to deliver the compositions to a subject. An inhalation apparatus, as used herein, is any device for administering an aerosol, such as dry powdered form of the compositions. This type of equipment is well known in the art and has been described in detail, such as that description found in Remington: The Science and Practice of Pharmacy, 19^{th} Edition, 1995, Mac Publishing Company, Easton, Pa., pages 1676-1692. Many U.S. patents also describe inhalation devices, such as U.S. Pat. No. 6,116,237.

“Powder” as used herein refers to a composition that consists of finely dispersed solid particles. Preferably the compositions are relatively free flowing and capable of being dispersed in an inhalation device and subsequently inhaled by a subject so that the compositions reach the lungs to permit penetration into the alveoli. A “dry powder” refers to a powder composition that has a moisture content such that the particles are readily dispersible in an inhalation device to form an aerosol. The moisture content is generally below about 10% by weight (% w) water, and in some embodiments is below about 5% w and preferably less than about 3% w. The powder may be formulated with polymers or optionally may be formulated with other materials such as liposomes, albumin and/or other carriers.

Aerosol dosage and delivery systems may be selected for a particular therapeutic application by one of skill in the art, such as described, for example in Gonda, I. “Aerosols for delivery of therapeutic and diagnostic agents to the respiratory tract,” in Critical Reviews in Therapeutic Drug Carrier Systems, 6:273-313 (1990), and in Moren, “Aerosol dosage forms and formulations,” in Aerosols in Medicine. Principles, Diagnosis and Therapy, Moren, et al., Eds., Esevier, Amsterdam, 1985.

The compositions, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compositions in water-soluble form. Additionally, suspensions of the active compositions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compositions to allow for the preparation of highly concentrated solutions.

Alternatively, the active compositions may be in powder form for constitution with a suitable vehicle, e.g., sterile pyrogen-free water, before use.

The compositions may also be formulated in rectal or vaginal compositions such as suppositories or retention enemas, e.g., containing conventional suppository bases such as cocoa butter or other glycerides.

In addition to the formulations described previously, the compositions may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also may include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

In some embodiments, Gemcitabine packaged inside the VLPs described herein, may be protected from deamination in the plasma during in vivo delivery. In certain embodiments, delivery of Gemcitabine through VLPs in vivo to a subject reduces drug induced side-effects.

In some embodiments, VLP preparations of the invention may be administered to a subject in combination (e.g., simultaneously or separately but as part of the same therapeutic regimen) with a compound or other pharmaceutical preparation for treating one or more conditions or diseases described herein. Accordingly, aspects of the invention relate to combination preparations or kits that contain one or more VLPs of the invention along with one or more separate compounds (e.g., drugs) that are not packaged in a VLP.

### EXAMPLES

### Example 1

In a non-limiting example, a VLP is produced such that it is modified to be optimized for loading siRNA. The VLP contains either wild-type coat proteins that are already positively charged, or coat proteins engineered to carry additional positive charges, e.g., in the N-terminal region of amino acids 1-26. Some VLPs are optimized for targeted delivery and a targeting peptide is fused in frame to be expressed in one or more of the surface exposed loops of the VLP. In one example, the targeting peptide is RGD. Coat proteins are produced in E. coli and P. pastoris. Modified coat proteins are purified. Some coat proteins are chemically linked to hyaluronic acid to mask immunogenic sites on the VLP. Self-assembly of the modified coat proteins is measured. Immune reactivity of the resulting VLP is measured in a mouse model system. VLPs are loaded with siRNA molecules and these are delivered to a mouse model in vivo. A reporter gene mouse model (GFP, luciferase, beta-galactosidase) is used and siRNA that are delivered in vivo are siRNAs directed against reporter genes. Loss of expression of the reporter gene is measured. Specific cellular targeting is followed in vivo via immunostaining and in vivo imaging techniques.

### Example 2

In some non-limiting examples, anti-growth activity is first measured in vitro in cell culture systems of transformed breast cancer cell lines. Successful delivery of siRNA molecules targeting oncogenes by the drug-loaded VLPs to the cells is measured by colony forming assays, cell cycle analysis, and/or measurement of apoptosis by FACS, immunofluorescence, immunoblotting, and/or colorimetric assays. In further non-limiting examples, the anti-cancer activity of target siRNA molecules directed against oncogenes is tested in cancer mouse models. Survival curves are prepared and tumor spreading and tumor mass is monitored by sacrificing and dissecting mice at regular intervals.

Some VLPs, genetically optimized to interact with Gemcitabine are loaded with Gemcitabine by swelling the VLP at pH 6.5 and trapping the solubilized Gemcitabine in the VLP by lowering the pH to pH 5.0. VLPs are administered to a pancreatic xenograph mouse model via oral and injection routes. Cytotoxicity is tested in vivo. Cancer progression is carefully monitored.

Some VLPs are modified to express targeting molecules specific for the integrin receptor and are loaded with Gemcitabine. Some coat proteins that express the integrin receptor-specific molecule are additionally chemically modified by PEGylation to reduce immunogenicity. These coat proteins are self-assembled and loaded with Gemcitabine. Cytotoxicity tests and anti-cell growth effects are monitored in vitro using pancreatic tumor cell lines. Cytotoxicity and anti-tumor effects, as well as specific targeting efficiency are analyzed in vivo using a pancreatic xenograph mouse model.

Some VLPs are modified to express MUC-1 antibody as a fusion protein and loaded with Gemcitabine. Cytotoxicity tests and anti-cell growth effects are monitored in vitro using breast cancer cell lines. Cytotoxicity and anti-tumor effects, as well as specific targeting efficiency are analyzed in vivo using a breast cancer xenograph mouse model.

In some examples, a VLP is loaded with cisplatinum and/or similar molecules.

### Example 3

In a non-limiting example, empty particles of Cowpea chlorotic mottle virus (CCMV) which can be used to encapsidate drug molecules and which can be targeted to defined cells were produced. Expression of the empty particles was undertaken in the yeast, Pischia pastoris, since this system has been previously used successfully for the production of empty CCMV particles. The anti-cancer drug Gemcitabine is encapsidated in the CCMV particles. The RGD-4C peptide, which binds integrins, was integrated into surface exposed loops of the viral coat protein and was expressed on the surface of the CCMV capsids. CCMV capsids expressing the RGD-4C peptide will be used to target cells which express integrins.

### Production of a Synthetic CCMV Coat Protein Gene

To produce a version of the CCMV coat protein (CP) which can be easily modified on both its outer (to incorporate cell targeting sequences) and inner (to optimise drug binding) surfaces, a synthetic gene was made by Geneart (Regensburg, Germany) (FIG. 1). The gene was so designed as to allow the removal of the N-terminal section, which controls RNA encapsidation and is on the inner surface of the assembled particles, and the insertion of targeting sequences in either the βC-αCD1 or βF-βG loops on the outer surface of the virus-like particles (VLPs). Accordingly, a wild-type CCMV coat protein sequence is similar to that shown in FIG. 1B, but it includes R, P, and K at positions 26, 75, and 131 respectively, instead of the substituions to H, G, and R shown for those positions in FIG. 1B.

The positions of the inserts are shown in FIG. 2.

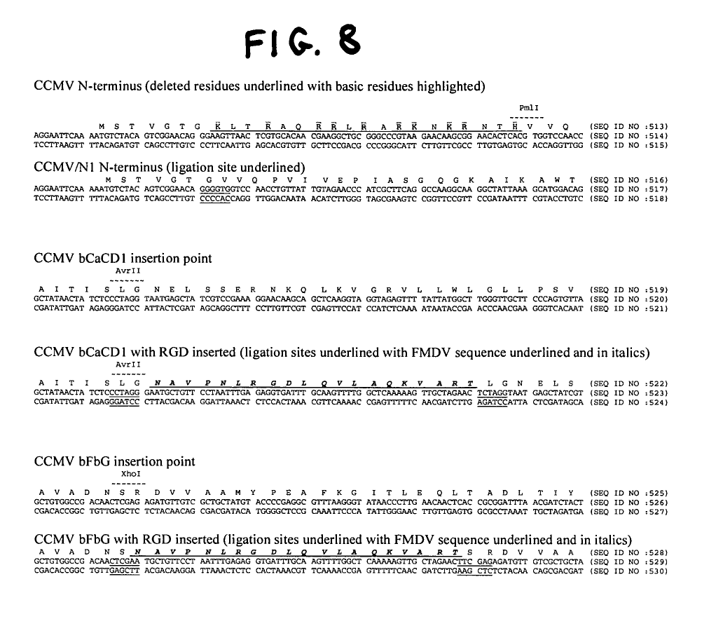

For the deletion, the first 7 amino acids (MSTVGTG, SEQ ID NO: 506) were added back after deleting the first 26 amino acids, effectively deleting residues 8-26 (the region which contains all 8 of the basic residues). The deletion and the insertions into the bCaCDI and bFbG loops (CCMV CP insertions) are shown in FIG. 8. The RGD peptide used has the following nucleic acid and amino acid sequence:

FMDV-O-20mer (RGD peptide):

| | |
|---|---|
| N A V P N L R G D L Q V L A Q K V A R T | (SEQ ID NO: 505) |
| AAT GCT GTT CCT AAT TTG AGA GGT GAT TTG CAA GTT TTG GCT CAA AAA GTT GCT AGA ACT | (SEQ ID NO: 508) |
| TTA CGA CAA GGA TTA AAC TCT CCA CTA AAC GTT CAA AAC CGA GTT TTT CAA CGA TCT TGA | (SEQ ID NO: 509) |

Expression of Synthetic Untargeted wt and N1 CP Genes in Pischia pastoris

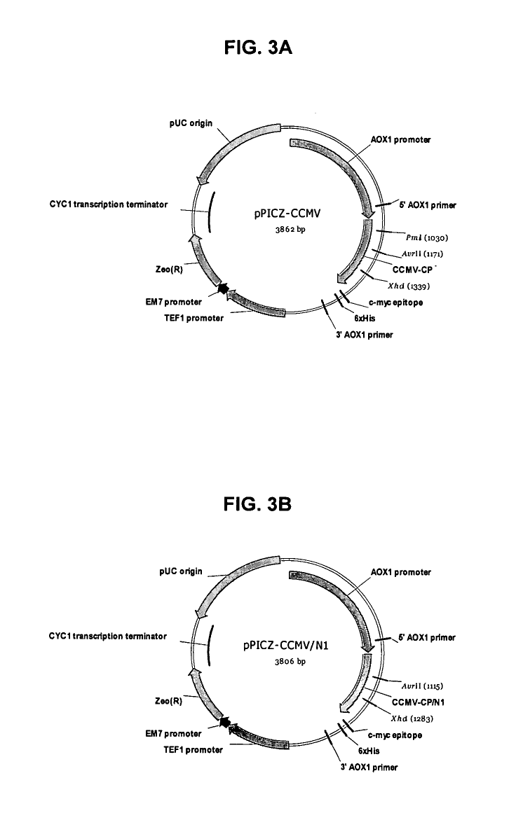

For expression of the CCMV coat proteins in Pischia pastoris, the EasySelect™ Pischia expression kit (Invitrogen, Carlsbad, Calif.) was used. The sequences encoding the full-length and CCMV/N1 mutant CPs were inserted into the multiple cloning site of plasmid pPICZ-A to give plasmids pPICZ-CCMV and pPICZ-CCMV/N1, respectively (FIG. 3). P. pastoris strain X-33 was used for expression. All liquid growth was at 30° C. and 250 rpm in baffled flasks. Cultures were started in 8 ml in MGY and grown overnight (up to 24 hours) in falcon tubes, leading to OD₆₀₀ of about 8.0. Cells were collected by centrifugation at 2000 g for 5 minutes and re-suspended in 32 ml of MM with 0.5% methanol in 250 ml flasks. 16 to 24 hours later the cells were harvested by centrifugation at 10 minutes at 3500 g.

Extraction/Purification of wt and N1 CCMV Particles from Pischia pastoris

The following method was used:

Re-suspend pellet in 3-9 ml of 0.2M NaOAc (pH4.8); combine smaller volumes for extraction in the cell disruptor (takes 10 ml); 3 passes at 30 KPSI on cell disruptor; clear cell debris at 12 000 g for 10 minutes; collect particles by centrifugation at 118 000 g for 2 hours 15 minutes; re-suspend in 50 mM NaOAc (pH4.8); for three 32 ml cultures resuspended to a collective 9 ml about 30 μg of wt and N1 particles were obtained.
Characterisation of wt and N1 Particles Produced in Pischia pastoris

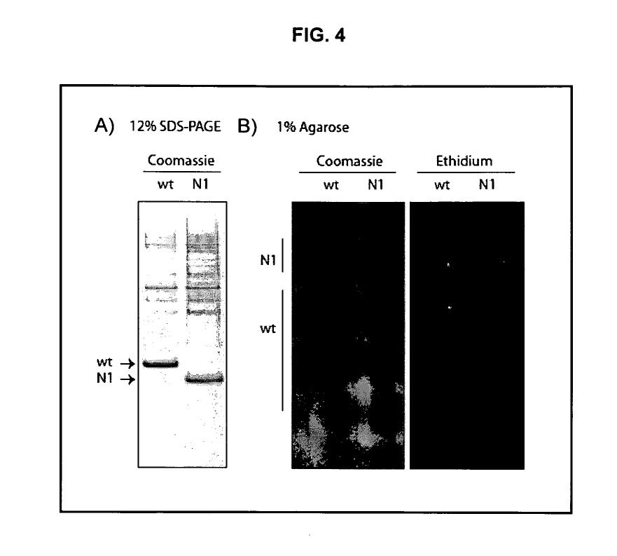

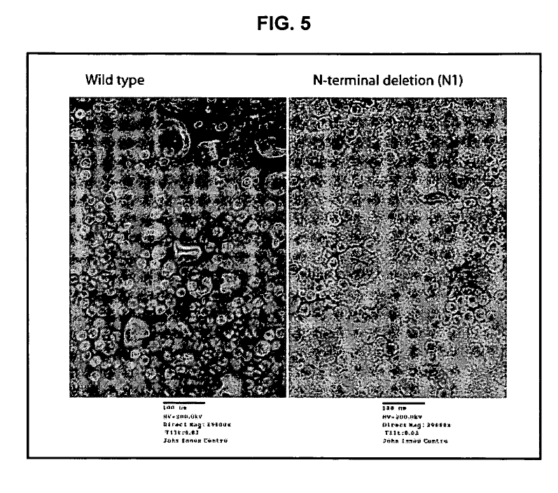

Approximately 1 μg samples of wt and N1 particles were denatured and examined by SDS/PAGE on a 12% polyacrylamide gel. Staining with Coomassie blue revealed prominent bands in the correct positions for the wt and N1 coat proteins (FIG. 4). Some contaminating proteins were also seen suggesting that further purification might be required in some embodiments. Analysis by non-denaturing agarose gel electrophoresis was used to examine 5 μg samples intact particles. To detect protein, the gels were stained with Coomassie blue. As is usual with this technique, the samples ran as a broad band in each case. However, the N1 sample ran more slowly than the wt, consistent with the particles having a different charge. When the gel was stained with ethidium bromide to reveal any encapsidated nucleic acid, only the wt sample gave a signal. wt CCMV CP is known to encapsidate heterogeneous RNAs. When the N-terminal sequence is deleted, as in the case of N1, heterogeneous RNAs is no longer encapsidated leading to particles that are likely entirely free of nucleic acid contaminants and therefore better suited as delivery vehicles, e.g., for RNAi molecules. FIG. 4 shows that no nucleic acid is detected in the N1 variant based on eithidium staining (whereas nucleic acid is present in the wt particle). TEM confirmed the presence of CCMV-like particles in both the wt and N1 samples (FIG. 5). Accordingly, N1 and similar variants do not contain detectable viral RNA or nucleic acid from the host cell. These particles are empty of viral or host RNA or DNA and can be used directly to load one or more agents of interest.

### Insertion of Targeting Peptides

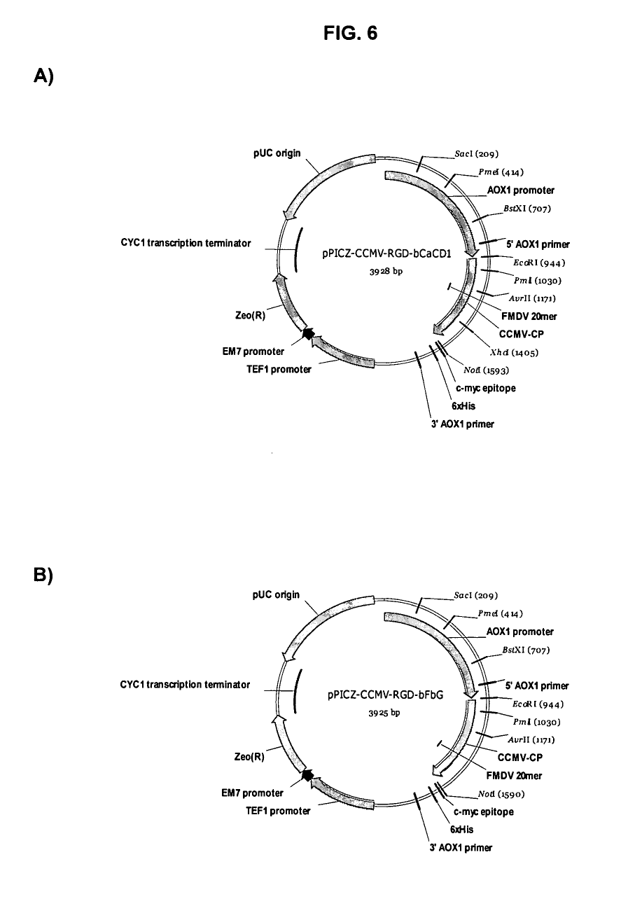

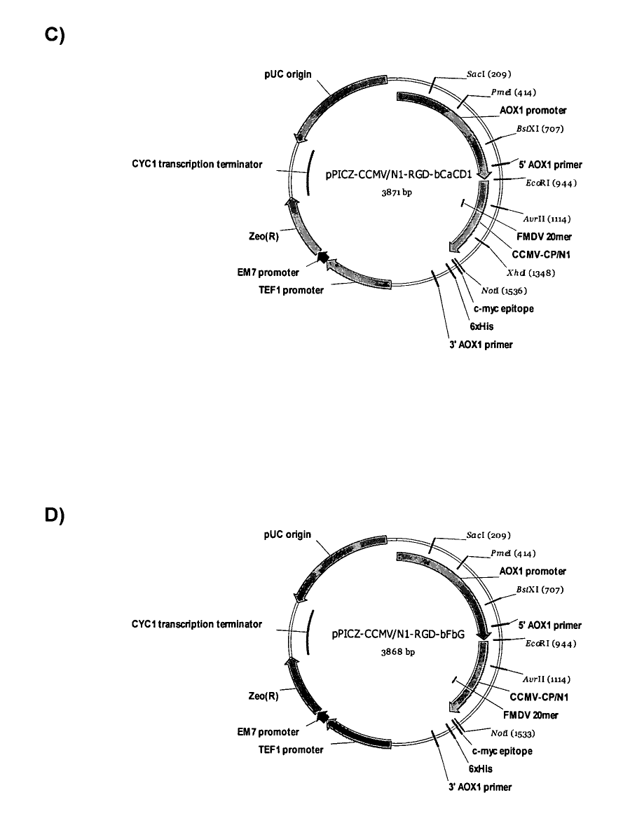

Oligonucleotides encoding the RGD-4C peptide were inserted into the βC-αCD1 or βF-βG loop of full-length or the N1 mutant of the CCMV CP to give the four plasmids shown in FIG. 6. These plasmids were used to transform P. pastoris. Only those colonies containing plasmid pPICZ-CCMV-RGD-bCaCD1, containing the RGD-4C peptide inserted into the βC-αCD1 site of the wt CP grew at an appreciable rate and produced detectable CCMV CP. This construct was used for further analysis.

Attempts to purify particles produced by pPICZ-CCMV-RGD-bCaCD1 using the CCMV purification protocol (applied successfully to wt and N1 CP) were unsuccessful as the particles were lost in the first clearing spin. This was attributed to a change in charge on the particle surface (caused by the basic nature of the RGD-containing peptide) causing particle aggregation. Attempts were made to prevent aggregation by carrying out the extractions at a higher pH and/or creating “mosaic” in which only a proportion of the 180 CP molecules in the particle bear the RGD-sequence. To investigate these possibilities, a revised extraction protocol was applied to Psichia expressing the N1 CP and the pPICZ-CCMV-RGD-bCaCD1 CP. The extracts were then mixed in various proportions: 6:0, 5:1, 4:2, 3:3, 2:4, 1:5, 0:6.

Solubilisation of particles at higher pH and creations of Mosaics:

The following method was used:

Disruption as described herein but in 50 mM Tris (pH 7.5) with 0.5 M CaCl₂ and 1 mM DTT plus 200 μM PMSF; incubate 60 minutes on ice with occasional mixing; clear cell debris at 12 000 g for 10 minutes; mix supernatants at various ratios for mosaic particles and incubate a further 30 minutes on ice; dialyse overnight against 0.1M NaOAc (pH 4.8) with 0.1M NaCl and 200 μM PMSF; dialyse a further 6 hours against 50 mM NaOAc (pH4.8); precipitate forms; clarify with 15 minutes at 20 000 g; collect particles from supernatant with 2 hours and 15 minutes at 118 000 g.

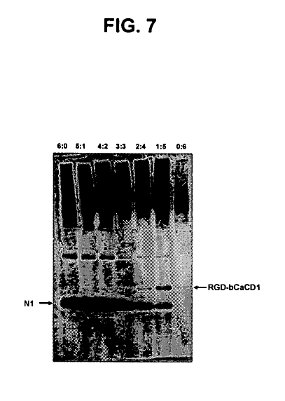

The particles were then examined by SDS/PAGE and western blotting using the anti-CCMV rabbit antibody PVAS-299. The N1 mutant and the pPICZ-CCMV-RGD-bCaCD1 CPs can readily be distinguished by their sizes (FIG. 7).

The results showed that both types of coat protein can be extracted with higher pH buffer but expression/extraction appears to be more efficient in the case of N1 (Sample 6:0) than pPICZ-CCMV-RGD-bCaCD1 (Sample 0:6). When the two types of CPs were reassembled in ratios shown above each lane, both types of coat protein could be seen in the sample. When N1 and pPICZ-CCMV-RGD-bCaCD1 extracts were mixed in a ratio of 1:5 prior to assembly, approximately equal amounts of the two CPs can be found in the samples. This is consistent with the expression/extraction of N1 being more efficient than pPICZ-CCMV-RGD-bCaCD1. The resulting preparation may be analyzed to determine the extent to which mosaic VLP and/or mixtures of two types of particles, those containing exclusively N1 CP and those containing exclusively pPICZ-CCMV-RGD-bCaCD1, or all three different particles are present in the preparation. This analysis may be performed, for example, by differentially labeling the different coat proteins and determining the extent to which the different coat proteins (e.g., as determined by measuring the different labels) are present in the same particles. It should be appreciated that the preparation may be homogeneous in some embodiments, and only contain mosaic VLP having the same ratio of different coat proteins. In some embodiments, a preparation may be heterogeneous and contain a mixture of different types of mosaic VLP, each type having a different ratio of different coat proteins. It should be appreciated that since the preparations containing pPICZ-CCMV-RGD-bCACD1 alone rapidly precipitate out of solution, at a minimum, the presence of the N1 CP is providing a solubilising effect, whether present as separate particles or in mosaic particles comprising both CP types.

All publications, patents and sequence database entries mentioned herein are hereby incorporated by reference in their entirety as if each individual publication or patent was specifically and individually indicated to be incorporated by reference. In case of conflict, the present application, including any definitions herein, will control.

What is claimed is:

1-91. (canceled)

92. A virus-like particle (VLP) preparation comprising:
(a) a mosaic VLP of two or more different CCMV coat proteins, wherein at least one of the coat proteins is modified to include a targeting peptide that comprises an integrin-binding motif, and wherein
(i) at least one of the coat proteins has a N-terminal deletion within the first 26 amino acids and wherein the deletion is of 1 to 26 amino acids in length,
(ii) at least one of the coat proteins comprises an amino acid sequence of a bacteriophage coat protein or functional portion thereof,
(iii) at least one of the coat proteins comprises one or more amino acid substitutions within the first 26 N-terminal amino acids and/or the coat protein comprises one or more amino acid substitutions and/or amino acid deletions within amino acids 52-176 of the coat protein,
(iv) at least one of the coat proteins comprises a moiety selected from the group consisting of polyethylene glycol (PEG), hyaluronic acid, a natural or synthetic polymer, a histidine tag, folic acid, a second targeting peptide not comprising an integrin-binding sequence, an antibody or functional fragment thereof, and a receptor ligand molecule,
(v) at least one of the coat proteins comprises an amino acid sequence that interacts selectively with a nucleic acid motif that is present on a heterologous RNA molecule;
(b) a heterologous RNA molecule, wherein the heterologous RNA molecule is a microRNA (miRNA), a short interfering RNA (siRNA), a double-stranded RNA (dsRNA), a short hairpin RNA (shRNA), RNAu, or an antisense RNA molecule; and
(c) a therapeutic molecule, wherein the therapeutic molecule is a therapeutic agent, a diagnostic agent or an imaging agent present in the interior of the assembled mosaic VLP.

93. The VLP preparation of claim 92, wherein the bacteriophage coat protein is selected from the group consisting of: bacteriophage MS2 coat protein and bacteriophage Qbeta coat protein.

94. The VLP preparation of claim 93, further comprising a heterologous RNA molecule, wherein the heterologous RNA molecule comprises a sequence selected from the group consisting of: MS2 hairpin/translational operator (TR) and Qbeta hairpin/translational operator (TR).

95. The VLP preparation of claim 92, wherein the integrin-binding motif comprises a RGD amino acid sequence.

96. The VLP preparation of claim 92, wherein the targeting peptide is fused to the modified coat protein as part of a chimeric protein.

97. The VLP preparation of claim 92, wherein the targeting peptide is chemically attached, directly or indirectly, to the modified coat protein.

98. The VLP preparation of claim 92, wherein the moiety is chemically attached, directly or indirectly, to the modified coat protein.

99. The VLP preparation of claim 92, wherein the moiety is a targeting peptide that is fused to the modified coat protein as part of a chimeric protein.

100. The VLP preparation of claim 92, wherein the moiety reduces immunogenicity of the VLP.

101. The VLP preparation of claim 92, wherein the therapeutic molecule is selected from the group consisting of: an anti-cancer drug, an antibiotic, an anti-viral agent, an anti-microbial agent, an anti-inflammatory agent, and an immunostimulatory agent.

102. The VLP preparation of claim 92, wherein the integrin targeting peptide directs the VLP to a tumor.

103. The VLP preparation of claim 92, further comprising a heterologous nucleic acid molecule.

104. The VLP preparation of claim 103, wherein the heterologous nucleic acid molecule is an expression vector for a gene.

105. The VLP preparation of claim 92, wherein the modified coat protein comprises a modification that promotes its interaction with a heterologous therapeutic or diagnostic molecule.

106. The VLP preparation of claim 92, wherein the integrin expressed by the cells or tissues is an av integrin.

107. The VLP preparation of claim 92, wherein the N-terminal deletion within the first 26 amino acids is a deletion of amino acids 8-26.

108. The VLP preparation of claim 92, wherein the modified coat protein is encoded by a synthetic DNA wherein the coding sequence is optimized for expression in a host cell.

109. The host cell of claim 108, wherein the host cell is a mammalian cell, a bacteria, or a yeast.

110. The VLP preparation of claim 109, wherein the coat protein comprises one or more amino acid substitutions within amino acids 52-176 of the coat protein, and wherein the substitution promotes direct or indirect attachment of a moiety selected from the group consisting of: polyethylene glycol (PEG), hyaluronic acid, a natural or synthetic polymer, a histidine tag, folic acid, a second targeting peptide not comprising an integrin-binding sequence, an antibody or functional fragment thereof, and a receptor ligand molecule.

111. The VLP preparation of claim 92, comprising at least two different CCMV coat proteins that are present in a relative ratio of 1:1, 2:1, 3:1, 4:1, 5:1, 6:1, 7:1, 8:1, 9:1, or 10:1, or at a higher ratio.

112. The VLP preparation of claim 111, wherein the coat protein that is present at a higher level in the VLP preparation forms a more stable VLP alone, and/or self-assembles alone more efficiently to form a VLP.

113. A pharmaceutical composition comprising:
(a) a mosaic VLP of two or more different CCMV coat proteins, wherein at least one of the coat proteins is modified to include a targeting peptide that comprises an integrin-binding motif, and wherein
(i) at least one of the coat proteins has a N-terminal deletion within the first 26 amino acids and the deletion is of 1 to 26 amino acids in length,
(ii) at least one of the coat proteins comprises an amino acid sequence of a bacteriophage coat protein or functional portion thereof,
(iii) at least one of the coat proteins comprises one or more amino acid substitutions within the first 26 N-terminal amino acids and/or the coat protein comprises one or more amino acid substitutions and/or amino acid deletions within amino acids 52-176 of the coat protein,
(iv) at least one of the coat proteins comprises a moiety selected from the group consisting of polyethylene glycol (PEG), hyaluronic acid, a natural or synthetic polymer, a histidine tag, folic acid, a second targeting peptide not comprising an integrin-binding sequence, an antibody or functional fragment thereof, and a receptor ligand molecule,
(v) at least one of the coat proteins comprises an amino acid sequence that interacts selectively with a nucleic acid motif that is present on a heterologous RNA molecule;
(b) a heterologous RNA molecule, wherein the heterologous RNA molecule is a microRNA (miRNA), a short interfering RNA (siRNA), a double-stranded RNA (dsRNA), a short hairpin RNA (shRNA), RNAu, or an antisense RNA molecule; and
(c) a therapeutic molecule, wherein the therapeutic molecule is a therapeutic agent, a diagnostic agent or an imaging agent present in the interior of the assembled mosaic VLP.

114. The pharmaceutical composition of claim 113, wherein the one or more additional modifications are selected from the group consisting of: (a) a N-terminal deletion within the first 26 amino acids and wherein the deletion is of 1 to 26 amino acids in length; (b) an N-terminal substitution, wherein the substitution comprises an amino acid sequence of a bacteriophage coat protein or functional portion thereof; (c) an amino acid substitution within the first 26 N-terminal amino acids; (d) an amino acid substitutions and/or amino acid deletion within amino acids 52-176 of the coat protein; and (e) an addition of a moiety selected from the group consisting of polyethylene glycol (PEG), hyaluronic acid, a natural or synthetic polymer, a histidine tag, folic acid, a second targeting peptide not comprising an integrin-binding sequence, an antibody or functional fragment thereof, and a receptor ligand molecule.


SEQUENCE LISTING (530 sequences)

SEQ ID NO: 1 - protein, 26 aa, artificial sequence
  synthetic polypeptide
MSTVGTGKLTRAQRRAAARKNKRNTR

SEQ ID NO: 2 - protein, 26 aa, artificial sequence
  synthetic polypeptide
MSTVGTGXLTXAQXXAAAXXNXXNTX

SEQ ID NO: 3 - protein, 26 aa, artificial sequence
  synthetic polypeptide
MSTVGTGKLTRAQRRAAARKNKRNTR

SEQ ID NO: 4 - protein, 26 aa, artificial sequence
  synthetic polypeptide
MSTVGTGKLTKAQKKAAAKKNKKNTK

SEQ ID NO: 5 - protein, 26 aa, artificial sequence
  synthetic polypeptide
MSTVGTGRLTRAQRRAAARRNRRNTR

SEQ ID NO: 6 - protein, 26 aa, artificial sequence
  synthetic polypeptide
MSTVGTGKLTAAQAGAAAAGNAANTG

SEQ ID NO: 7 - protein, 26 aa, artificial sequence
  synthetic polypeptide
MSTVGTGALTRAQRGAAAVKNKLNTI

SEQ ID NO: 8 - protein, 26 aa, artificial sequence
  synthetic polypeptide
MSTVGTGDLTDAQDDAAADDNDDNTD

SEQ ID NO: 9 - protein, 26 aa, artificial sequence
  synthetic polypeptide
MSTVGTGELTEAQEEAAAEENEENTE

SEQ ID NO: 10 - protein, 26 aa, artificial sequence
  synthetic polypeptide
MSTVGTGDLTEAQAGAAALINVANTG

SEQ ID NO: 11 - protein, 26 aa, artificial sequence
  synthetic polypeptide
MSTVGTGKLTRAQEDAAARKNDENTA

SEQ ID NO: 12 - protein, 26 aa, artificial sequence
  synthetic polypeptide
MSTVGTGYLTWAQFFAAAYYNIINTW

SEQ ID NO: 13 - protein, 26 aa, artificial sequence
  synthetic polypeptide
MSTVGTGWLTRAQYRAAAFKNKWNTR

SEQ ID NO: 14 - protein, 26 aa, artificial sequence
  synthetic polypeptide
MSTVGTGKLTRAQEDAAAWKNFYNTR

SEQ ID NO: 15 - protein, 26 aa, artificial sequence
  synthetic polypeptide
MSTVGTGHLTCAQRRAAACKNKRNTH

SEQ ID NO: 16 - protein, 26 aa, artificial sequence
  synthetic polypeptide
MSTVGTGELTDAQHEAAADCNHHNTD

SEQ ID NO: 17 - protein, 26 aa, artificial sequence
  synthetic polypeptide
MSTVGTGCLTHAQAVAAARKNIGNTW

SEQ ID NO: 18 - protein, 7 aa, artificial sequence
  synthetic polypeptide
CAAAEAK

SEQ ID NO: 19 - protein, 4 aa, artificial sequence
  synthetic polypeptide
ISLP

SEQ ID NO: 20 - protein, 7 aa, artificial sequence
  synthetic polypeptide
LPSVSGT

SEQ ID NO: 21 - protein, 7 aa, artificial sequence
  synthetic polypeptide
NSKDVVA

SEQ ID NO: 22 - protein, 8 aa, artificial sequence
  synthetic polypeptide
SAALTEGD

SEQ ID NO: 23 - RNA, 19 nt, artificial sequence
  synthetic polynucleotide
uaaaaucuuccugcccacc

SEQ ID NO: 24 - RNA, 19 nt, artificial sequence
  synthetic polynucleotide
ggaagcuguuggcugaaaa

SEQ ID NO: 25 - RNA, 19 nt, artificial sequence
  synthetic polynucleotide
cacauccagcccaucuguc

SEQ ID NO: 26 - RNA, 19 nt, artificial sequence
  synthetic polynucleotide
ccgugcuccuggggcuggg

SEQ ID NO: 27 - RNA, 19 nt, artificial sequence
  synthetic polynucleotide
ggaguuggaucucucagaa

SEQ ID NO: 28 - DNA, 19 bp, artificial sequence
  synthetic polynucleotide
aagtcccgctcattacaaa

SEQ ID NO: 29 - RNA, 21 nt, artificial sequence
  synthetic polynucleotide
aagaccagccucuuugcccag

SEQ ID NO: 30 - RNA, 19 nt, artificial sequence
  synthetic polynucleotide
ggaccaggcagaaaacgag

SEQ ID NO: 31 - RNA, 17 nt, artificial sequence
  synthetic polynucleotide
cuaucaggaugacgcgg

SEQ ID NO: 32 - RNA, 21 nt, artificial sequence
  synthetic polynucleotide
aaccaccugggccaguauuau

SEQ ID NO: 33 - RNA, 21 nt, artificial sequence
  synthetic polynucleotide
ugacacaggcaggcuugacuu

SEQ ID NO: 34 - RNA, 21 nt, artificial sequence
  synthetic polynucleotide
aagcucaauucggacaacaag

SEQ ID NO: 35 - RNA, 21 nt, artificial sequence
  synthetic polynucleotide
aaguccuguaugagugggaac

SEQ ID NO: 36 - DNA, 59 bp, artificial sequence
  synthetic polynucleotide
tcgacgaacatctacaacgcctgcttcaagagagcaggcgttgtagatgttcttttttt

SEQ ID NO: 37 - DNA, 59 bp, artificial sequence
  synthetic polynucleotide
tcgaccaatgacaagagccgtgacttcaagagagtcacggctcttgtcattgttttttt

SEQ ID NO: 38 - DNA, 19 bp, artificial sequence
  synthetic polynucleotide
ggtgaagaagggcgtccaa

SEQ ID NO: 39 - DNA, 62 bp, artificial sequence
  synthetic polynucleotide
gatccgttggagctgttggcgtagttcaagagactacgccaacagctccaactttttggaaa

SEQ ID NO: 40 - DNA, 20 bp, artificial sequence
  synthetic polynucleotide
aggtggtgttaacagcagag

SEQ ID NO: 41 - DNA, 20 bp, artificial sequence
  synthetic polynucleotide
aatgttccaatgccccactc

SEQ ID NO: 42 - RNA, 21 nt, artificial sequence
  synthetic polynucleotide
aacugccaguggccagggccu

SEQ ID NO: 43 - RNA, 21 nt, artificial sequence
  synthetic polynucleotide
aaccugcggucuggagucaac

SEQ ID NO: 44 - RNA, 21 nt, artificial sequence
  synthetic polynucleotide
uaauccaauucgaagaccaau

SEQ ID NO: 45 - DNA, 21 bp, artificial sequence
  synthetic polynucleotide
aggggcagcaactgatgaaaa

SEQ ID NO: 46 - DNA, 21 bp, artificial sequence
  synthetic polynucleotide
aaggcatcgcagaaggtttat

SEQ ID NO: 47 - RNA, 19 nt, artificial sequence
  synthetic polynucleotide
ucgaaguacucagcguaag

SEQ ID NO: 48 - RNA, 19 nt, artificial sequence
  synthetic polynucleotide
gaaccugaggaccaagaac

SEQ ID NO: 49 - DNA, 29 bp, artificial sequence
  synthetic polynucleotide
aatgttctcttccaggtcagccctgtctc

SEQ ID NO: 50 - RNA, 21 nt, artificial sequence
  synthetic polynucleotide
aagaaguacgagaagcuggag

SEQ ID NO: 51 - RNA, 21 nt, artificial sequence
  synthetic polynucleotide
aagcaagcuguagaacacgua

SEQ ID NO: 52 - RNA, 21 nt, artificial sequence
  synthetic polynucleotide
aagccgugcaaagaacucuuu

SEQ ID NO: 53 - RNA, 21 nt, artificial sequence
  synthetic polynucleotide
aacaguagguuuguagugagc

SEQ ID NO: 54 - RNA, 21 nt, artificial sequence
  synthetic polynucleotide
aaaggggcauccacagacauc

SEQ ID NO: 55 - DNA, 20 bp, artificial sequence
  synthetic polynucleotide
ttattcttctctttggagga

SEQ ID NO: 56 - DNA, 19 bp, artificial sequence
  synthetic polynucleotide
actggacttccagaagaac

SEQ ID NO: 57 - DNA, 21 bp, artificial sequence
  synthetic polynucleotide
aattgcacgtaatcacccaaa

SEQ ID NO: 58 - DNA, 21 bp, artificial sequence
  synthetic polynucleotide
aagaaatacaaggaagatgag

SEQ ID NO: 59 - RNA, 19 nt, artificial sequence
  synthetic polynucleotide
gacaugcuugcucauaguc

SEQ ID NO: 60 - RNA, 19 nt, artificial sequence
  synthetic polynucleotide
gaccugccuccucaucguc

SEQ ID NO: 61 - DNA, 21 bp, artificial sequence
  synthetic polynucleotide
aaggtggagcaagcggtggag

SEQ ID NO: 62 - DNA, 21 bp, artificial sequence
  synthetic polynucleotide
aaggagttgaaggcctacaaa

SEQ ID NO: 63 - RNA, 19 nt, artificial sequence
  synthetic polynucleotide
ugccuacgaacucuucacc

SEQ ID NO: 64 - RNA, 19 nt, artificial sequence
  synthetic polynucleotide
uauggagcugcagaggaug

SEQ ID NO: 65 - DNA, 19 bp, artificial sequence
  synthetic polynucleotide
ttggccaagccacacacag

SEQ ID NO: 66 - DNA, 19 bp, artificial sequence
  synthetic polynucleotide
gttctcagccattgctagc

SEQ ID NO: 67 - DNA, 19 bp, artificial sequence
  synthetic polynucleotide
ggcagcatgtattgctgag

SEQ ID NO: 68 - RNA, 21 nt, artificial sequence
  synthetic polynucleotide
aauccaggacucauuccagau

SEQ ID NO: 69 - RNA, 21 nt, artificial sequence
  synthetic polynucleotide
aagugaagaauacgaucaagu

SEQ ID NO: 70 - RNA, 21 nt, artificial sequence
  synthetic polynucleotide
aacaacgaguaccucaacccu

SEQ ID NO: 71 - RNA, 21 nt, artificial sequence
  synthetic polynucleotide
aacgcuugagcuggaaguaag

SEQ ID NO: 72 - RNA, 19 nt, artificial sequence
  synthetic polynucleotide
ccuucaagcuucugaucuc

SEQ ID NO: 73 - RNA, 21 nt, artificial sequence
  synthetic polynucleotide
aacuucgacuuugucaccgag

SEQ ID NO: 74 - RNA, 22 nt, artificial sequence
  synthetic polynucleotide
aagcacugcagagacauggaag

SEQ ID NO: 75 - DNA, 21 bp, artificial sequence
  synthetic polynucleotide
aacagccatggatacacttga

SEQ ID NO: 76 - DNA, 20 bp, artificial sequence
  synthetic polynucleotide
aatgacaaagaggcagcagg

SEQ ID NO: 77 - DNA, 20 bp, artificial sequence
  synthetic polynucleotide
aacctgccacactcaagatc

SEQ ID NO: 78 - DNA, 21 bp, artificial sequence
  synthetic polynucleotide
agctgaacttcaggagctgcc

SEQ ID NO: 79 - DNA, 21 bp, artificial sequence
  synthetic polynucleotide
aagcctttcgcaagttcctga

SEQ ID NO: 80 - DNA, 20 bp, artificial sequence
  synthetic polynucleotide
acggcataggcgatgaggag

SEQ ID NO: 81 - DNA, 20 bp, artificial sequence
  synthetic polynucleotide
aggaaggccgggtgattgtg

SEQ ID NO: 82 - DNA, 19 bp, artificial sequence
  synthetic polynucleotide
gtctggtacgactggagta

SEQ ID NO: 83 - DNA, 20 bp, artificial sequence
  synthetic polynucleotide
gacagctttaggcacctcta

SEQ ID NO: 84 - RNA, 19 nt, artificial sequence
  synthetic polynucleotide
ggauucgaacugcacuucu

SEQ ID NO: 85 - RNA, 19 nt, artificial sequence
  synthetic polynucleotide
acuguggcacccagcuugu

SEQ ID NO: 86 - RNA, 19 nt, artificial sequence
  synthetic polynucleotide
gcccaaagugaaucccuuc

SEQ ID NO: 87 - DNA, 47 bp, artificial sequence
  synthetic polynucleotide
tttgaatatctgtgctgagaacacagttctcagcacagatattcttt

SEQ ID NO: 88 - DNA, 49 bp, artificial sequence
  synthetic polynucleotide
tttgtcaattagctggaacatcacagatgttccagctaattgacttttt

SEQ ID NO: 89 - DNA, 29 bp, artificial sequence
  synthetic polynucleotide
aatgagaaaagcaaaaggtgccctgtctc

SEQ ID NO: 90 - RNA, 22 nt, artificial sequence
  synthetic polynucleotide
caucgaugugugugagaacugc

SEQ ID NO: 91 - RNA, 22 nt, artificial sequence
  synthetic polynucleotide
cuguucucagcuggagaagcuu

SEQ ID NO: 92 - RNA, 22 nt, artificial sequence
  synthetic polynucleotide
ggagccuucaugguaagggauu

SEQ ID NO: 93 - DNA, 15 bp, artificial sequence
  synthetic polynucleotide
ggcaccatgaaggcg

SEQ ID NO: 94 - RNA, 19 nt, artificial sequence
  synthetic polynucleotide
cugggcuguacuuuguaua

SEQ ID NO: 95 - RNA, 21 nt, artificial sequence
  synthetic polynucleotide
auguagccuggagauccauuu

SEQ ID NO: 96 - RNA, 21 nt, artificial sequence
  synthetic polynucleotide
ccucagggcagagaaccaucu

SEQ ID NO: 97 - RNA, 21 nt, artificial sequence
  synthetic polynucleotide
cuggacuuccagaagaacauc

SEQ ID NO: 98 - RNA, 21 nt, artificial sequence
  synthetic polynucleotide
aagauccgccagagccccucg

SEQ ID NO: 99 - DNA, 21 bp, artificial sequence
  synthetic polynucleotide
aacagggactcacgtgaagct

SEQ ID NO: 100 - DNA, 21 bp, artificial sequence
  synthetic polynucleotide
aagacctgtttgatctgatcc

SEQ ID NO: 101 - RNA, 19 nt, artificial sequence
  synthetic polynucleotide
uagacugacccagcuggaa

SEQ ID NO: 102 - RNA, 19 nt, artificial sequence
  synthetic polynucleotide
gaugcaauuacacaacaga

SEQ ID NO: 103 - RNA, 19 nt, artificial sequence
  synthetic polynucleotide
cucaggagaggagccauuu

SEQ ID NO: 104 - RNA, 19 nt, artificial sequence
  synthetic polynucleotide
gauugaagacacaggaggc

SEQ ID NO: 105 - RNA, 19 nt, artificial sequence
  synthetic polynucleotide
gcaacucuggaugggauug

SEQ ID NO: 106 - RNA, 19 nt, artificial sequence
  synthetic polynucleotide
ggaguucaugagugcuaua

SEQ ID NO: 107 - RNA, 19 nt, artificial sequence
  synthetic polynucleotide
cagaggaaccugcuggcga

SEQ ID NO: 108 - DNA, 21 bp, artificial sequence
  synthetic polynucleotide
aactggcaacctccagagaat

SEQ ID NO: 109 - DNA, 21 bp, artificial sequence
  synthetic polynucleotide
aagagacctcgtggagaaact

SEQ ID NO: 110 - DNA, 21 bp, artificial sequence
  synthetic polynucleotide
aacagtagaggagccgtcaaa

SEQ ID NO: 111 - RNA, 19 nt, artificial sequence
  synthetic polynucleotide
gggugagaccaucuucauc

SEQ ID NO: 112 - RNA, 19 nt, artificial sequence
  synthetic polynucleotide
ggccaaagucuaugaagau

SEQ ID NO: 113 - RNA, 21 nt, artificial sequence
  synthetic polynucleotide
aaucaucaucaagaaagggca

SEQ ID NO: 114 - RNA, 19 nt, artificial sequence
  synthetic polynucleotide
aauugcuggagcuggccuu

SEQ ID NO: 115 - DNA, 19 bp, artificial sequence
  synthetic polynucleotide
gaatgaagatcgatagtaa

SEQ ID NO: 116 - DNA, 19 bp, artificial sequence
  synthetic polynucleotide
gcacaggtgtagcaagtaa

SEQ ID NO: 117 - DNA, 19 bp, artificial sequence
  synthetic polynucleotide
ggagaattatgctttgaaa

SEQ ID NO: 118 - DNA, 19 bp, artificial sequence
  synthetic polynucleotide
gcagtgctttgcagtatga

SEQ ID NO: 119 - DNA, 19 bp, artificial sequence
  synthetic polynucleotide
cagaaagccttgcgagttt

SEQ ID NO: 120 - DNA, 19 bp, artificial sequence
  synthetic polynucleotide
gaatttggattgccacaga

SEQ ID NO: 121 - DNA, 19 bp, artificial sequence
  synthetic polynucleotide
gaaggtgttcccactgata

SEQ ID NO: 122 - DNA, 19 bp, artificial sequence
  synthetic polynucleotide
gagagggtcctgcaaagaa

SEQ ID NO: 123 - RNA, 19 nt, artificial sequence
  synthetic polynucleotide
ugaugaaaaugagcaccag

SEQ ID NO: 124 - DNA, 21 bp, artificial sequence
  synthetic polynucleotide
aaaatccctgccagaaccacc

SEQ ID NO: 125 - DNA, 21 bp, artificial sequence
  synthetic polynucleotide
aacaagaccttcgactcttcc

SEQ ID NO: 126 - DNA, 19 bp, artificial sequence
  synthetic polynucleotide
aaacgaaagcgagtacact

SEQ ID NO: 127 - DNA, 21 bp, artificial sequence
  synthetic polynucleotide
agatggacttctctgtacagg

SEQ ID NO: 128 - DNA, 21 bp, artificial sequence
  synthetic polynucleotide
gacattgtctgtctctgagga

SEQ ID NO: 129 - RNA, 19 nt, artificial sequence
  synthetic polynucleotide
ggacuuauccuggcuagag

SEQ ID NO: 130 - RNA, 19 nt, artificial sequence
  synthetic polynucleotide
ccggaucuacucgacuccc

SEQ ID NO: 131 - RNA, 19 nt, artificial sequence
  synthetic polynucleotide
ccuaaucacacacucugua

SEQ ID NO: 132 - RNA, 19 nt, artificial sequence
  synthetic polynucleotide
acugcagacaaaacaccuu

SEQ ID NO: 133 - RNA, 19 nt, artificial sequence
  synthetic polynucleotide
uccaaccucuggguccguu

SEQ ID NO: 134 - RNA, 19 nt, artificial sequence
  synthetic polynucleotide
acugugaccguuucugugu

SEQ ID NO: 135 - RNA, 19 nt, artificial sequence
  synthetic polynucleotide
cucagacucuacagauugc

SEQ ID NO: 136 - RNA, 19 nt, artificial sequence
  synthetic polynucleotide
gggcaaggccuugcagcuc

SEQ ID NO: 137 - RNA, 19 nt, artificial sequence
  synthetic polynucleotide
augacugucaggauguugc

SEQ ID NO: 138 - RNA, 19 nt, artificial sequence
  synthetic polynucleotide
gcaacauccugacagucau

SEQ ID NO: 139 - RNA, 19 nt, artificial sequence
  synthetic polynucleotide
ugaacggugcugucaugua

SEQ ID NO: 140 - RNA, 19 nt, artificial sequence
  synthetic polynucleotide
gcagagguucggcaugaau

SEQ ID NO: 141 - DNA, 21 bp, artificial sequence
  synthetic polynucleotide
aagatgttcgtggacctgaac

SEQ ID NO: 142 - DNA, 38 bp, artificial sequence
  synthetic polynucleotide
aagatgattgttcgtccctgctatagtgagtcgtatta

SEQ ID NO: 143 - RNA, 21 nt, artificial sequence
  synthetic polynucleotide
gaacgaauccugaagacaucu

SEQ ID NO: 144 - RNA, 19 nt, artificial sequence
  synthetic polynucleotide
gaaggaagcuuugcuagcu

SEQ ID NO: 145 - DNA, 29 bp, artificial sequence
  synthetic polynucleotide
aagcctggctacagcaatatgcctgtctc

SEQ ID NO: 146 - RNA, 19 nt, artificial sequence
  synthetic polynucleotide
ggcucauuugcacucaauu

SEQ ID NO: 147 - RNA, 19 nt, artificial sequence
  synthetic polynucleotide
gccacaaaucugauaguau

SEQ ID NO: 148 - RNA, 19 nt, artificial sequence
  synthetic polynucleotide
cuggacuuccagaagaaca

SEQ ID NO: 149 - RNA, 19 nt, artificial sequence
  synthetic polynucleotide
ugaccaucaccgaguuuau

SEQ ID NO: 150 - DNA, 17 bp, artificial sequence
  synthetic polynucleotide
gattataggaaatgttg

SEQ ID NO: 151 - DNA, 21 bp, artificial sequence
  synthetic polynucleotide
aagaaggcagatgaggggtta

SEQ ID NO: 152 - DNA, 21 bp, artificial sequence
  synthetic polynucleotide
aacaaagatggaccaacacag

SEQ ID NO: 153 - RNA, 19 nt, artificial sequence
  synthetic polynucleotide
ggacgaggucugcgugaau

SEQ ID NO: 154 - RNA, 21 nt, artificial sequence
  synthetic polynucleotide
aagaaacgcgguaaucggacu

SEQ ID NO: 155 - DNA, 45 bp, artificial sequence
  synthetic polynucleotide
gcacaaagagcttgctcccttcaagagagagcaagctctttgtgc

SEQ ID NO: 156 - DNA, 47 bp, artificial sequence
  synthetic polynucleotide
gtccgggaagctgaaagtcttcaagagagactttcagcttcccggac

SEQ ID NO: 157 - DNA, 47 bp, artificial sequence
  synthetic polynucleotide
gagatctggacagaatcgcttcaagagagcgattctgtccagatctc

SEQ ID NO: 158 - DNA, 19 bp, artificial sequence
  synthetic polynucleotide
ggagaacagcattaaactg

SEQ ID NO: 159 - DNA, 19 bp, artificial sequence
  synthetic polynucleotide
ggtcaacattctgatgtct

SEQ ID NO: 160 - DNA, 19 bp, artificial sequence
  synthetic polynucleotide
gggcaaggccttgcagctc

SEQ ID NO: 161 - DNA, 21 bp, artificial sequence
  synthetic polynucleotide
gtacaatgatgacatccgtaa

SEQ ID NO: 162 - DNA, 21 bp, artificial sequence
  synthetic polynucleotide
gtacgtccgcgggttgctgca

SEQ ID NO: 163 - RNA, 21 nt, artificial sequence
  synthetic polynucleotide
aagcauguggccugcuaugga

SEQ ID NO: 164 - DNA, 65 bp, artificial sequence
  synthetic polynucleotide
gatcccgcctcaacaacaacaacaacttcaagagagttgttgttgttgttgaggttttttggaaa

SEQ ID NO: 165 - RNA, 21 nt, artificial sequence
  synthetic polynucleotide
aagaagagcuucgagacuuuc

SEQ ID NO: 166 - DNA, 21 bp, artificial sequence
  synthetic polynucleotide
aactgggagagtacggtttcc

SEQ ID NO: 167 - DNA, 21 bp, artificial sequence
  synthetic polynucleotide
aagtcggacgcaacagagaaa

SEQ ID NO: 168 - RNA, 21 nt, artificial sequence
  synthetic polynucleotide
ggccaaccagaugcggcuguu

SEQ ID NO: 169 - RNA, 21 nt, artificial sequence
  synthetic polynucleotide
gauguuugacauccucuucuu

SEQ ID NO: 170 - RNA, 21 nt, artificial sequence
  synthetic polynucleotide
ggcugcaagcaguauuuacuu

SEQ ID NO: 171 - RNA, 21 nt, artificial sequence
  synthetic polynucleotide
ggacagagucagauuacaguu

SEQ ID NO: 172 - DNA, 23 bp, artificial sequence
  synthetic polynucleotide
agccactgtggaagaagtatatt

SEQ ID NO: 173 - RNA, 19 nt, artificial sequence
  synthetic polynucleotide
cccucugguuggugauuca

SEQ ID NO: 174 - RNA, 19 nt, artificial sequence
  synthetic polynucleotide
ggaugauggcaccuuccua

SEQ ID NO: 175 - DNA, 21 bp, artificial sequence
  synthetic polynucleotide
aattggagatgaagatgtagg

SEQ ID NO: 176 - RNA, 19 nt, artificial sequence
  synthetic polynucleotide
caguuccgccacuugccaa

SEQ ID NO: 177 - RNA, 19 nt, artificial sequence
  synthetic polynucleotide
agccugguggacauuuauu

SEQ ID NO: 178 - RNA, 19 nt, artificial sequence
  synthetic polynucleotide
gaugugugaaacucugaac

SEQ ID NO: 179 - RNA, 19 nt, artificial sequence
  synthetic polynucleotide
cagauggguaaggauggca

SEQ ID NO: 180 - RNA, 21 nt, artificial sequence
  synthetic polynucleotide
aagcacuaucauugcgaaucc

SEQ ID NO: 181 - DNA, 21 bp, artificial sequence
  synthetic polynucleotide
aactgggcgagtattacatga

SEQ ID NO: 182 - DNA, 19 bp, artificial sequence
  synthetic polynucleotide
tgaacaaagtgagagacat

SEQ ID NO: 183 - DNA, 21 bp, artificial sequence
  synthetic polynucleotide
aatgtgtgaatgacaactact

SEQ ID NO: 184 - DNA, 21 bp, artificial sequence
  synthetic polynucleotide
aatacggactcaccttgcttg

SEQ ID NO: 185 - RNA, 20 nt, artificial sequence
  synthetic polynucleotide
ccgccgcuuccauuuuuccu

SEQ ID NO: 186 - RNA, 21 nt, artificial sequence
  synthetic polynucleotide
aggaaaaauggaagcggcggg

SEQ ID NO: 187 - RNA, 19 nt, artificial sequence
  synthetic polynucleotide
ccuggaacucaccuaccug

SEQ ID NO: 188 - RNA, 19 nt, artificial sequence
  synthetic polynucleotide
cuaccuuucuacggacgug

SEQ ID NO: 189 - RNA, 19 nt, artificial sequence
  synthetic polynucleotide
gauccggaaguacacgaug

SEQ ID NO: 190 - RNA, 19 nt, artificial sequence
  synthetic polynucleotide
gaaggaauccuccaaguca

SEQ ID NO: 191 - DNA, 21 bp, artificial sequence
  synthetic polynucleotide
aagctccatgtcacagtacga

SEQ ID NO: 192 - DNA, 21 bp, artificial sequence
  synthetic polynucleotide
aagcgctgcgtcatgaatgtt

SEQ ID NO: 193 - DNA, 21 bp, artificial sequence
  synthetic polynucleotide
ctgcctaaggcggatttgaat

SEQ ID NO: 194 - DNA, 21 bp, artificial sequence
  synthetic polynucleotide
ggcaggcgacgagtttgaact

SEQ ID NO: 195 - DNA, 21 bp, artificial sequence
  synthetic polynucleotide
gtgcgtggaaagcgtagacaa

SEQ ID NO: 196 - DNA, 21 bp, artificial sequence
  synthetic polynucleotide
ggcggagttcacagctctata

SEQ ID NO: 197 - DNA, 21 bp, artificial sequence
  synthetic polynucleotide
gtgggcataagtgctgatcta

SEQ ID NO: 198 - DNA, 21 bp, artificial sequence
  synthetic polynucleotide
ctcggtcctgcgattattaat

SEQ ID NO: 199 - DNA, 53 bp, artificial sequence
  synthetic polynucleotide
aaggccgagatcagcaaagttcaagagactttgctgatctcggcctttttttt

SEQ ID NO: 200 - RNA, 19 nt, artificial sequence
  synthetic polynucleotide
ggccagccaucacaaucaa

SEQ ID NO: 201 - RNA, 19 nt, artificial sequence
  synthetic polynucleotide
ggaacuucuaaaugaacca

SEQ ID NO: 202 - RNA, 19 nt, artificial sequence
  synthetic polynucleotide
gguccaguuaagguuuugu

SEQ ID NO: 203 - RNA, 19 nt, artificial sequence
  synthetic polynucleotide
gguuaccuuccaccuacaa

SEQ ID NO: 204 - RNA, 21 nt, artificial sequence
  synthetic polynucleotide
aaguggaugccuuucggguca

SEQ ID NO: 205 - RNA, 21 nt, artificial sequence
  synthetic polynucleotide
aaggagaacaguucuugcggc

SEQ ID NO: 206 - RNA, 21 nt, artificial sequence
  synthetic polynucleotide
aagguccagucauuccaaaug

SEQ ID NO: 207 - RNA, 20 nt, artificial sequence
  synthetic polynucleotide
aggaaggaccuguugaccuu

SEQ ID NO: 208 - RNA, 19 nt, artificial sequence
  synthetic polynucleotide
uggugagcuuaaugaauga

SEQ ID NO: 209 - RNA, 19 nt, artificial sequence
  synthetic polynucleotide
agaggacauugagguguau

SEQ ID NO: 210 - DNA, 23 bp, artificial sequence
  synthetic polynucleotide
aactgctcaacaccggaattttt

SEQ ID NO: 211 - RNA, 22 nt, artificial sequence
  synthetic polynucleotide
ccauccuguacaacuucaugug

SEQ ID NO: 212 - RNA, 19 nt, artificial sequence
  synthetic polynucleotide
caauaaggaagaagcccuu

SEQ ID NO: 213 - RNA, 21 nt, artificial sequence
  synthetic polynucleotide
nnauuccuucuucgggaaguc

SEQ ID NO: 214 - RNA, 19 nt, artificial sequence
  synthetic polynucleotide
ggcuggcuucauccacugc

SEQ ID NO: 215 - DNA, 19 bp, artificial sequence
  synthetic polynucleotide
cgaagatgttgacctggtc

SEQ ID NO: 216 - DNA, 19 bp, artificial sequence
  synthetic polynucleotide
agagttgtcctgtagttcg

SEQ ID NO: 217 - RNA, 19 nt, artificial sequence
  synthetic polynucleotide
ggaaauaugggaaagaucc

SEQ ID NO: 218 - RNA, 19 nt, artificial sequence
  synthetic polynucleotide
ggagacuuucaaauacauc

SEQ ID NO: 219 - DNA, 21 bp, artificial sequence
  synthetic polynucleotide
aaccttctggaacccgcccac

SEQ ID NO: 220 - DNA, 21 bp, artificial sequence
  synthetic polynucleotide
aatgttgcagaggggaaggag

SEQ ID NO: 221 - DNA, 21 bp, artificial sequence
  synthetic polynucleotide
aagagaatggagcacatgaaa

SEQ ID NO: 222 - DNA, 21 bp, artificial sequence
  synthetic polynucleotide
aagatgagcataaccagtgac

SEQ ID NO: 223 - DNA, 21 bp, artificial sequence
  synthetic polynucleotide
aaggccctatatttgcattaa

SEQ ID NO: 224 - DNA, 23 bp, artificial sequence
  synthetic polynucleotide
gttggctgagtgctatgggctga

SEQ ID NO: 225 - DNA, 22 bp, artificial sequence
  synthetic polynucleotide
gggcttatggaggaccctatga

SEQ ID NO: 226 - DNA, 52 bp, artificial sequence
  synthetic polynucleotide
ggaacagcagagaagctcattcaagagatgagcttctctgctgttccttttt

SEQ ID NO: 227 - DNA, 51 bp, artificial sequence
  synthetic polynucleotide
caccgaagcagcacgacttcttcttcaagagagaagaagtcgtgctgcttc

SEQ ID NO: 228 - RNA, 20 nt, artificial sequence
  synthetic polynucleotide
agguucagcagcuccacgga

SEQ ID NO: 229 - DNA, 63 bp, artificial sequence
  synthetic polynucleotide
gatcccccctcggggatactgtctgattcaagagacagacagtatccccgaggtttttggaaa

SEQ ID NO: 230 - DNA, 19 bp, artificial sequence
  synthetic polynucleotide
gagcatcttcgagcaagaa

SEQ ID NO: 231 - DNA, 19 bp, artificial sequence
  synthetic polynucleotide
catgtggcaccgtttgcct

SEQ ID NO: 232 - RNA, 19 nt, artificial sequence
  synthetic polynucleotide
uggcuuaucuuacacugga

SEQ ID NO: 233 - RNA, 25 nt, artificial sequence
  synthetic polynucleotide
ggcucaaguucaggagugagaacaa

SEQ ID NO: 234 - RNA, 25 nt, artificial sequence
  synthetic polynucleotide
ggaacgacuucaaagagaacuugag

SEQ ID NO: 235 - RNA, 25 nt, artificial sequence
  synthetic polynucleotide
gcauuacaaccagacaguugauauu

SEQ ID NO: 236 - RNA, 18 nt, artificial sequence
  synthetic polynucleotide
guggagcagcggcaaaau

SEQ ID NO: 237 - RNA, 19 nt, artificial sequence
  synthetic polynucleotide
uccucauuugaggaauaaa

SEQ ID NO: 238 - RNA, 19 nt, artificial sequence
  synthetic polynucleotide
ggaauaaaccuugugcagu

SEQ ID NO: 239 - RNA, 19 nt, artificial sequence
  synthetic polynucleotide
uaaaccuugugcaguugua

SEQ ID NO: 240 - RNA, 19 nt, artificial sequence
  synthetic polynucleotide
ccuugugcaguuguacagu

SEQ ID NO: 241 - RNA, 21 nt, artificial sequence
  synthetic polynucleotide
ccggcaaccucgcggccaauu

SEQ ID NO: 242 - RNA, 21 nt, artificial sequence
  synthetic polynucleotide
cgacuaccaucugcacuuauu

SEQ ID NO: 243 - DNA, 21 bp, artificial sequence
  synthetic polynucleotide
aaccggctctccattggcatt

SEQ ID NO: 244 - DNA, 21 bp, artificial sequence
  synthetic polynucleotide
gtggttggaaatggcaccttt

SEQ ID NO: 245 - RNA, 21 nt, artificial sequence
  synthetic polynucleotide
aagcgaccaggauuauauucu

SEQ ID NO: 246 - RNA, 21 nt, artificial sequence
  synthetic polynucleotide
aagugagguggaaaggcguuu

SEQ ID NO: 247 - DNA, 55 bp, artificial sequence
  synthetic polynucleotide
tcccatgtccacttcgactatgatgtcaagagcatcatagtcgaagtggacattt

SEQ ID NO: 248 - DNA, 51 bp, artificial sequence
  synthetic polynucleotide
tcccagatctacttcttccgaggtcaagagcctcggaagaagtagatcttt

SEQ ID NO: 249 - DNA, 55 bp, artificial sequence
  synthetic polynucleotide
tcccaggatgctgatggctatgccttcaagagaggcatagccatcagcatccttt

SEQ ID NO: 250 - DNA, 21 bp, artificial sequence
  synthetic polynucleotide
aatgcccatctctataggttt

SEQ ID NO: 251 - RNA, 19 nt, artificial sequence
  synthetic polynucleotide
ggauaugcgaagaaagauc

SEQ ID NO: 252 - DNA, 21 bp, artificial sequence
  synthetic polynucleotide
aaggaggaagctgatgagaac

SEQ ID NO: 253 - DNA, 21 bp, artificial sequence
  synthetic polynucleotide
aacgtccggaacaaactgaag

SEQ ID NO: 254 - DNA, 21 bp, artificial sequence
  synthetic polynucleotide
aagtggtacgcagattgcacg

SEQ ID NO: 255 - DNA, 21 bp, artificial sequence
  synthetic polynucleotide
aaggagaagaaaaagaaggac

SEQ ID NO: 256 - DNA, 19 bp, artificial sequence
  synthetic polynucleotide
gcaggtagagttggctttg

SEQ ID NO: 257 - DNA, 19 bp, artificial sequence
  synthetic polynucleotide
gactatgatcgactgcggc

SEQ ID NO: 258 - DNA, 19 bp, artificial sequence
  synthetic polynucleotide
gtgagaacgatgagaaata

SEQ ID NO: 259 - DNA, 19 bp, artificial sequence
  synthetic polynucleotide
gttggtgcctaatcactta

SEQ ID NO: 260 - DNA, 19 bp, artificial sequence
  synthetic polynucleotide
gacgtgtcaggaccttcgt

SEQ ID NO: 261 - DNA, 19 bp, artificial sequence
  synthetic polynucleotide
cttaactggtgtacattaa

SEQ ID NO: 262 - RNA, 23 nt, artificial sequence
  synthetic polynucleotide
caauaugaucauccaguccauua

SEQ ID NO: 263 - RNA, 23 nt, artificial sequence
  synthetic polynucleotide
caagcaugcagcuucuaccguuc

SEQ ID NO: 264 - RNA, 23 nt, artificial sequence
  synthetic polynucleotide
cagucgaguucucccaccgcucu

SEQ ID NO: 265 - RNA, 23 nt, artificial sequence
  synthetic polynucleotide
gagaccaucuacgaccugggcac

SEQ ID NO: 266 - RNA, 23 nt, artificial sequence
  synthetic polynucleotide
gagagugacauggcgccuguccu

SEQ ID NO: 267 - RNA, 23 nt, artificial sequence
  synthetic polynucleotide
aaggaaccaaacaguugaaacug

SEQ ID NO: 268 - RNA, 23 nt, artificial sequence
  synthetic polynucleotide
gagucuucuaucgcucccaucgu

SEQ ID NO: 269 - RNA, 23 nt, artificial sequence
  synthetic polynucleotide
aaauugagguggauucacgaguu

SEQ ID NO: 270 - RNA, 23 nt, artificial sequence
  synthetic polynucleotide
uaguaucagcgaggaacaaauua

SEQ ID NO: 271 - RNA, 23 nt, artificial sequence
  synthetic polynucleotide
aagaguauauagcacaggauuua

SEQ ID NO: 272 - RNA, 23 nt, artificial sequence
  synthetic polynucleotide
aagauugcccuugcucgcaauaa

SEQ ID NO: 273 - RNA, 23 nt, artificial sequence
  synthetic polynucleotide
uaucaccguaugaaauggaaacu

SEQ ID NO: 274 - RNA, 23 nt, artificial sequence
  synthetic polynucleotide
aaguggaauggguaacucuucuu

SEQ ID NO: 275 - RNA, 23 nt, artificial sequence
  synthetic polynucleotide
aacggccgucccaaagcuggcug

SEQ ID NO: 276 - RNA, 23 nt, artificial sequence
  synthetic polynucleotide
aagaaacagauucugcgcuugau

SEQ ID NO: 277 - RNA, 23 nt, artificial sequence
  synthetic polynucleotide
gaauuagguccacuucaaugucc

SEQ ID NO: 278 - RNA, 23 nt, artificial sequence
  synthetic polynucleotide
aagauucuuccauuaaauugccu

SEQ ID NO: 279 - DNA, 21 bp, artificial sequence
  synthetic polynucleotide
aactaccagaaaggtatacct

SEQ ID NO: 280 - DNA, 21 bp, artificial sequence
  synthetic polynucleotide
aagacccttgtgctcgttgtc

SEQ ID NO: 281 - DNA, 21 bp, artificial sequence
  synthetic polynucleotide
aagcagagtgacctggtagat

SEQ ID NO: 282 - RNA, 19 nt, artificial sequence
  synthetic polynucleotide
ucacaguguccuuuaugua

SEQ ID NO: 283 - RNA, 19 nt, artificial sequence
  synthetic polynucleotide
auggcuucgacgaguucaa

SEQ ID NO: 284 - RNA, 19 nt, artificial sequence
  synthetic polynucleotide
gcaugaaccggaggcccau

SEQ ID NO: 285 - DNA, 21 bp, artificial sequence
  synthetic polynucleotide
agaccagagcaggaacaagtt

SEQ ID NO: 286 - DNA, 19 bp, artificial sequence
  synthetic polynucleotide
gaagatttgcgcagtggac

SEQ ID NO: 287 - RNA, 21 nt, artificial sequence
  synthetic polynucleotide
ugguucacugaagacccaguu

SEQ ID NO: 288 - RNA, 19 nt, artificial sequence
  synthetic polynucleotide
auuccugcucaauggauuu

SEQ ID NO: 289 - DNA, 23 bp, artificial sequence
  synthetic polynucleotide
tagagcctggctgggttgttttg

SEQ ID NO: 290 - DNA, 21 bp, artificial sequence
  synthetic polynucleotide
aaccacaacacaaagtcacag

SEQ ID NO: 291 - RNA, 22 nt, artificial sequence
  synthetic polynucleotide
aaaccggcaggaguuuacccag

SEQ ID NO: 292 - RNA, 21 nt, artificial sequence
  synthetic polynucleotide
aagguggccauuaaggugauu

SEQ ID NO: 293 - RNA, 19 nt, artificial sequence
  synthetic polynucleotide
ucagaagaccacaaucuac

SEQ ID NO: 294 - RNA, 19 nt, artificial sequence
  synthetic polynucleotide
gugaagucaacaugccugc

SEQ ID NO: 295 - RNA, 19 nt, artificial sequence
  synthetic polynucleotide
cacccuacacgaagccaga

SEQ ID NO: 296 - RNA, 19 nt, artificial sequence
  synthetic polynucleotide
ggacagaugaagcugcuuu

SEQ ID NO: 297 - DNA, 20 bp, artificial sequence
  synthetic polynucleotide
ggtggacaagaatgctagac

SEQ ID NO: 298 - DNA, 20 bp, artificial sequence
  synthetic polynucleotide
gaaggattgaccagttaacc

SEQ ID NO: 299 - DNA, 20 bp, artificial sequence
  synthetic polynucleotide
gcttgaacatgagcaagagc

SEQ ID NO: 300 - DNA, 21 bp, artificial sequence
  synthetic polynucleotide
aacttccggcagaaacttctg

SEQ ID NO: 301 - RNA, 19 nt, artificial sequence
  synthetic polynucleotide
gccacagcauacauccugu

SEQ ID NO: 302 - RNA, 19 nt, artificial sequence
  synthetic polynucleotide
cgcacgcuaaugcuggcau

SEQ ID NO: 303 - RNA, 18 nt, artificial sequence
  synthetic polynucleotide
aagcgguguuucucagaa

SEQ ID NO: 304 - RNA, 19 nt, artificial sequence
  synthetic polynucleotide
gucggagggaagacagugc

SEQ ID NO: 305 - RNA, 19 nt, artificial sequence
  synthetic polynucleotide
gcaugugauugcugacgcc

SEQ ID NO: 306 - DNA, 21 bp, artificial sequence
  synthetic polynucleotide
aaggcttcccaactgatgcaa

SEQ ID NO: 307 - DNA, 21 bp, artificial sequence
  synthetic polynucleotide
aagccaaggaagcattgggta

SEQ ID NO: 308 - DNA, 21 bp, artificial sequence
  synthetic polynucleotide
aagtactagaaggagaggtgg

SEQ ID NO: 309 - DNA, 21 bp, artificial sequence
  synthetic polynucleotide
gttcaaaagctggatgatctt

SEQ ID NO: 310 - DNA, 21 bp, artificial sequence
  synthetic polynucleotide
aagatacggtccagaagctta

SEQ ID NO: 311 - DNA, 21 bp, artificial sequence
  synthetic polynucleotide
ccagcggctcaaggaggtttt

SEQ ID NO: 312 - DNA, 21 bp, artificial sequence
  synthetic polynucleotide
gagtcgggaccacagtttatt

SEQ ID NO: 313 - DNA, 21 bp, artificial sequence
  synthetic polynucleotide
taggctaattgtactgctctt

SEQ ID NO: 314 - DNA, 21 bp, artificial sequence
  synthetic polynucleotide
ggagatcctctacaaagggtt

SEQ ID NO: 315 - DNA, 21 bp, artificial sequence
  synthetic polynucleotide
aagagatgctaatagcagttt

SEQ ID NO: 316 - DNA, 21 bp, artificial sequence
  synthetic polynucleotide
actcttactgctctccagttt

SEQ ID NO: 317 - DNA, 21 bp, artificial sequence
  synthetic polynucleotide
cgagacctatcgccgcatcgt

SEQ ID NO: 318 - DNA, 21 bp, artificial sequence
  synthetic polynucleotide
gggcggctttgccaagtgctt

SEQ ID NO: 319 - RNA, 19 nt, artificial sequence
  synthetic polynucleotide
cccugagaucccagcgcug

SEQ ID NO: 320 - DNA, 19 bp, artificial sequence
  synthetic polynucleotide
gatcaatggctacacagga

SEQ ID NO: 321 - RNA, 19 nt, artificial sequence
  synthetic polynucleotide
accugucgaggacuucauc

SEQ ID NO: 322 - RNA, 19 nt, artificial sequence
  synthetic polynucleotide
guacaagguggagcgcaac

SEQ ID NO: 323 - DNA, 19 bp, artificial sequence
  synthetic polynucleotide
cccggaaatttcccgtccc

SEQ ID NO: 324 - DNA, 19 bp, artificial sequence
  synthetic polynucleotide
gagatgacctgcattgccc

SEQ ID NO: 325 - RNA, 19 nt, artificial sequence
  synthetic polynucleotide
acucuugguucagaaagga

SEQ ID NO: 326 - DNA, 19 bp, artificial sequence
  synthetic polynucleotide
ggttcactactagtaaact

SEQ ID NO: 327 - DNA, 19 bp, artificial sequence
  synthetic polynucleotide
ggaatctactcgtttgtat

SEQ ID NO: 328 - RNA, 19 nt, artificial sequence
  synthetic polynucleotide
ccaagaaccagagaaaaga

SEQ ID NO: 329 - RNA, 19 nt, artificial sequence
  synthetic polynucleotide
cucuuuagaaacugggcaa

SEQ ID NO: 330 - DNA, 21 bp, artificial sequence
  synthetic polynucleotide
aagactccagtggtaatctac

SEQ ID NO: 331 - DNA, 19 bp, artificial sequence
  synthetic polynucleotide
tagagctacagaacgaaag

SEQ ID NO: 332 - DNA, 19 bp, artificial sequence
  synthetic polynucleotide
gaatgtgaacaccaccaaa

SEQ ID NO: 333 - DNA, 19 bp, artificial sequence
  synthetic polynucleotide
ctacacaaatattgaggat

SEQ ID NO: 334 - DNA, 19 bp, artificial sequence
  synthetic polynucleotide
ctgtacttccatacttgat

SEQ ID NO: 335 - DNA, 21 bp, artificial sequence
  synthetic polynucleotide
aagccaagacaaattctgtgt

SEQ ID NO: 336 - DNA, 21 bp, artificial sequence
  synthetic polynucleotide
aacctccgtgatgttgcttga

SEQ ID NO: 337 - DNA, 19 bp, artificial sequence
  synthetic polynucleotide
caaagccagaaacaagttg

SEQ ID NO: 338 - DNA, 19 bp, artificial sequence
  synthetic polynucleotide
aaataaactctacctggtt

SEQ ID NO: 339 - DNA, 19 bp, artificial sequence
  synthetic polynucleotide
aaacctcagaatctgctta

SEQ ID NO: 340 - DNA, 19 bp, artificial sequence
  synthetic polynucleotide
gttacttctatgcctgatt

SEQ ID NO: 341 - RNA, 21 nt, artificial sequence
  synthetic polynucleotide
gaccgagaagguagacaauug

SEQ ID NO: 342 - DNA, 21 bp, artificial sequence
  synthetic polynucleotide
aagaggcacaaggtccacatc

SEQ ID NO: 343 - RNA, 19 nt, artificial sequence
  synthetic polynucleotide
augcagcuggagauggcac

SEQ ID NO: 344 - RNA, 19 nt, artificial sequence
  synthetic polynucleotide
aggaaguuggaaggaucca

SEQ ID NO: 345 - RNA, 21 nt, artificial sequence
  synthetic polynucleotide
gaacacuuaucaagccuaauu

SEQ ID NO: 346 - RNA, 21 nt, artificial sequence
  synthetic polynucleotide
ggagagaacaagcauauuuuu

SEQ ID NO: 347 - RNA, 21 nt, artificial sequence
  synthetic polynucleotide
ugauguaccuauucucuuauu

SEQ ID NO: 348 - RNA, 21 nt, artificial sequence
  synthetic polynucleotide
gauaagagauuucagaagauu

SEQ ID NO: 349 - RNA, 19 nt, artificial sequence
  synthetic polynucleotide
gucaccacagcgcaangga

SEQ ID NO: 350 - RNA, 19 nt, artificial sequence
  synthetic polynucleotide
acucuagaugcucagacuu

SEQ ID NO: 351 - DNA, 21 bp, artificial sequence
  synthetic polynucleotide
aggatccatcttcctggttac

SEQ ID NO: 352 - RNA, 19 nt, artificial sequence
  synthetic polynucleotide
uaacaccagnacggacggg

SEQ ID NO: 353 - RNA, 19 nt, artificial sequence
  synthetic polynucleotide
ugcuccucucaugugggau

SEQ ID NO: 354 - RNA, 19 nt, artificial sequence
  synthetic polynucleotide
ucauuggucaguugucauc

SEQ ID NO: 355 - RNA, 19 nt, artificial sequence
  synthetic polynucleotide
gagacuugggcggucaaau

SEQ ID NO: 356 - RNA, 19 nt, artificial sequence
  synthetic polynucleotide
cggugacacucaguauguc

SEQ ID NO: 357 - RNA, 21 nt, artificial sequence
  synthetic polynucleotide
aagcaugaccagccugcuuac

SEQ ID NO: 358 - RNA, 19 nt, artificial sequence
  synthetic polynucleotide
gguuguccuugaguaauaa

SEQ ID NO: 359 - RNA, 21 nt, artificial sequence
  synthetic polynucleotide
gucugguacgacuggaguacc

SEQ ID NO: 360 - DNA, 21 bp, artificial sequence
  synthetic polynucleotide
aacattcactggtgcaactgc

SEQ ID NO: 361 - DNA, 21 bp, artificial sequence
  synthetic polynucleotide
aacaacatcttcctacatgag

SEQ ID NO: 362 - DNA, 21 bp, artificial sequence
  synthetic polynucleotide
aaagaattggatctggatcat

SEQ ID NO: 363 - RNA, 21 nt, artificial sequence
  synthetic polynucleotide
aauaguucagcaguuuggcua

SEQ ID NO: 364 - DNA, 21 bp, artificial sequence
  synthetic polynucleotide
gaatttaaaaccagaattatc

SEQ ID NO: 365 - DNA, 21 bp, artificial sequence
  synthetic polynucleotide
aagcgaagcagtggttcaggt

SEQ ID NO: 366 - RNA, 21 nt, artificial sequence
  synthetic polynucleotide
agacgagcugagcgagaagca

SEQ ID NO: 367 - RNA, 19 nt, artificial sequence
  synthetic polynucleotide
caucuacaagcccaacaac

SEQ ID NO: 368 - RNA, 19 nt, artificial sequence
  synthetic polynucleotide
cuucgacuuugucaccgag

SEQ ID NO: 369 - DNA, 21 bp, artificial sequence
  synthetic polynucleotide
aacctgtctccacaaagtgtg

SEQ ID NO: 370 - RNA, 19 nt, artificial sequence
  synthetic polynucleotide
caacaagaagacgcgaauc

SEQ ID NO: 371 - RNA, 21 nt, artificial sequence
  synthetic polynucleotide
aaucgccuaggaagacugauc

SEQ ID NO: 372 - RNA, 21 nt, artificial sequence
  synthetic polynucleotide
ccccuguagcggccaaacauu

SEQ ID NO: 373 - RNA, 21 nt, artificial sequence
  synthetic polynucleotide
caaaguacaaaggaauuuauu

SEQ ID NO: 374 - RNA, 21 nt, artificial sequence
  synthetic polynucleotide
cgggagggacagacuuucuuu

SEQ ID NO: 375 - DNA, 21 bp, artificial sequence
  synthetic polynucleotide
gtgaaccacaactccgtattc

SEQ ID NO: 376 - RNA, 21 nt, artificial sequence
  synthetic polynucleotide
aacugguugacgaaaguggug

SEQ ID NO: 377 - RNA, 21 nt, artificial sequence
  synthetic polynucleotide
aacccgcguuggcgugguuga

SEQ ID NO: 378 - RNA, 19 nt, artificial sequence
  synthetic polynucleotide
accaauccagcacccaucc

SEQ ID NO: 379 - RNA, 19 nt, artificial sequence
  synthetic polynucleotide
cugaugaccagcaacuuga

SEQ ID NO: 380 - RNA, 19 nt, artificial sequence
  synthetic polynucleotide
gagcugcaaggccuugccc

SEQ ID NO: 381 - DNA, 26 bp, artificial sequence
  synthetic polynucleotide
ctcctccattgtggaacccaaggagc

SEQ ID NO: 382 - RNA, 21 nt, artificial sequence
  synthetic polynucleotide
guauggacacugacucagauu

SEQ ID NO: 383 - RNA, 21 nt, artificial sequence
  synthetic polynucleotide
guacuuccagcuagauguuuu

SEQ ID NO: 384 - RNA, 19 nt, artificial sequence
  synthetic polynucleotide
gagcgaaaccugcucucag

SEQ ID NO: 385 - RNA, 19 nt, artificial sequence
  synthetic polynucleotide
gggugacuacuaccgcuac

SEQ ID NO: 386 - RNA, 19 nt, artificial sequence
  synthetic polynucleotide
agacagcacccucaucaug

SEQ ID NO: 387 - RNA, 19 nt, artificial sequence
  synthetic polynucleotide
guccugguggcgaguucga

SEQ ID NO: 388 - RNA, 19 nt, artificial sequence
  synthetic polynucleotide
cgucucuaugaccucaaca

SEQ ID NO: 389 - RNA, 21 nt, artificial sequence
  synthetic polynucleotide
augaagagucuuccaauccuu

SEQ ID NO: 390 - DNA, 21 bp, artificial sequence
  synthetic polynucleotide
aaggtccacttcgtatgctgg

SEQ ID NO: 391 - DNA, 21 bp, artificial sequence
  synthetic polynucleotide
aagggcaagtttcccgtgcag

SEQ ID NO: 392 - DNA, 21 bp, artificial sequence
  synthetic polynucleotide
aagaaggcagatgaggggtta

SEQ ID NO: 393 - RNA, 21 nt, artificial sequence
  synthetic polynucleotide
aaucaaaggcuaugucuggcg

SEQ ID NO: 394 - RNA, 21 nt, artificial sequence
  synthetic polynucleotide
aaggacuacgccgacucuauu

SEQ ID NO: 395 - DNA, 21 bp, artificial sequence
  synthetic polynucleotide
cgcctctttcccagtccatgt

SEQ ID NO: 396 - DNA, 21 bp, artificial sequence
  synthetic polynucleotide
gagctgagtgctcaggctaaa

SEQ ID NO: 397 - RNA, 19 nt, artificial sequence
  synthetic polynucleotide
aguggcccguuuccagcgg

SEQ ID NO: 398 - DNA, 21 bp, artificial sequence
  synthetic polynucleotide
aaggtgattggtagaggtgca

SEQ ID NO: 399 - DNA, 21 bp, artificial sequence
  synthetic polynucleotide
aagcacaaaagcttgtctcca

SEQ ID NO: 400 - RNA, 19 nt, artificial sequence
  synthetic polynucleotide
ggaauucauggccuuuguu

SEQ ID NO: 401 - RNA, 19 nt, artificial sequence
  synthetic polynucleotide
gaucucaaguuucaacacc

SEQ ID NO: 402 - RNA, 19 nt, artificial sequence
  synthetic polynucleotide
gucuguaaagaccaaggga

SEQ ID NO: 403 - RNA, 19 nt, artificial sequence
  synthetic polynucleotide
ggagcaaguaguggggcug

SEQ ID NO: 404 - RNA, 19 nt, artificial sequence
  synthetic polynucleotide
guacauccauuauaagcug

SEQ ID NO: 405 - DNA, 21 bp, artificial sequence
  synthetic polynucleotide
aacttccagctggcatatagg

SEQ ID NO: 406 - RNA, 21 nt, artificial sequence
  synthetic polynucleotide
aagggucaugcucuaucagau

SEQ ID NO: 407 - RNA, 21 nt, artificial sequence
  synthetic polynucleotide
aagaagacaucauccggaaua

SEQ ID NO: 408 - RNA, 21 nt, artificial sequence
  synthetic polynucleotide
aaggagauaccgugcggugcu

SEQ ID NO: 409 - DNA, 19 bp, artificial sequence
  synthetic polynucleotide
ccggacagttccatgtata

SEQ ID NO: 410 - RNA, 19 nt, artificial sequence
  synthetic polynucleotide
ggagaaaagccuuacagau

SEQ ID NO: 411 - RNA, 19 nt, artificial sequence
  synthetic polynucleotide
guauuuggccgccgacgca

SEQ ID NO: 412 - DNA, 21 bp, artificial sequence
  synthetic polynucleotide
aagactggagaaagtggcatg

SEQ ID NO: 413 - RNA, 21 nt, artificial sequence
  synthetic polynucleotide
aaucacuguggagacauuugc

SEQ ID NO: 414 - RNA, 21 nt, artificial sequence
  synthetic polynucleotide
aaugaagagggacacuucccu

SEQ ID NO: 415 - RNA, 21 nt, artificial sequence
  synthetic polynucleotide
aaccucgcugcaaagaaugug

SEQ ID NO: 416 - RNA, 21 nt, artificial sequence
  synthetic polynucleotide
aacuccaucuguucuccugac

SEQ ID NO: 417 - RNA, 21 nt, artificial sequence
  synthetic polynucleotide
aaaguuugcuuggcacaccuu

SEQ ID NO: 418 - DNA, 20 bp, artificial sequence
  synthetic polynucleotide
aaggcctaatgccgaacaca

SEQ ID NO: 419 - DNA, 21 bp, artificial sequence
  synthetic polynucleotide
aactttggctgccatcatcca

SEQ ID NO: 420 - RNA, 19 nt, artificial sequence
  synthetic polynucleotide
uaagcuccaagagaaaggc

SEQ ID NO: 421 - RNA, 19 nt, artificial sequence
  synthetic polynucleotide
gcccuaucccuuuacguca

SEQ ID NO: 422 - DNA, 39 bp, artificial sequence
  synthetic polynucleotide
gtggaccatgcactgcatgcctatagtgagtcgtattac

SEQ ID NO: 423 - DNA, 19 bp, artificial sequence
  synthetic polynucleotide
agatcctggctaactgttc

SEQ ID NO: 424 - DNA, 19 bp, artificial sequence
  synthetic polynucleotide
tacggactcaccttgcttg

SEQ ID NO: 425 - RNA, 19 nt, artificial sequence
  synthetic polynucleotide
cuggacacaguguguuuga

SEQ ID NO: 426 - RNA, 19 nt, artificial sequence
  synthetic polynucleotide
cugaugaccagcaacuuga

SEQ ID NO: 427 - RNA, 19 nt, artificial sequence
  synthetic polynucleotide
gcucuucgccauggacaca

SEQ ID NO: 428 - RNA, 19 nt, artificial sequence
  synthetic polynucleotide
gcgacagcuggaguaugaa

SEQ ID NO: 429 - RNA, 18 nt, artificial sequence
  synthetic polynucleotide
ccaugagcaccguucucc

SEQ ID NO: 430 - RNA, 19 nt, artificial sequence
  synthetic polynucleotide
cuugaccaaggagcucaac

SEQ ID NO: 431 - RNA, 19 nt, artificial sequence
  synthetic polynucleotide
ggagcucaacuucaccacc

SEQ ID NO: 432 - RNA, 19 nt, artificial sequence
  synthetic polynucleotide
cggccacucgcuuccgggc

SEQ ID NO: 433 - RNA, 19 nt, artificial sequence
  synthetic polynucleotide
gcuccgucgacugcgcgcc

SEQ ID NO: 434 - DNA, 21 bp, artificial sequence
  synthetic polynucleotide
aaaccaccgtggagctctact

SEQ ID NO: 435 - DNA, 21 bp, artificial sequence
  synthetic polynucleotide
aagagcccgaccttctgtgaa

SEQ ID NO: 436 - RNA, 19 nt, artificial sequence
  synthetic polynucleotide
guucuuccgccagauugug

SEQ ID NO: 437 - RNA, 21 nt, artificial sequence
  synthetic polynucleotide
cggacuacccuuagcacaauu

SEQ ID NO: 438 - RNA, 21 nt, artificial sequence
  synthetic polynucleotide
gaauagucaccauauucacuu

SEQ ID NO: 439 - DNA, 19 bp, artificial sequence
  synthetic polynucleotide
ggttccatcgaatcctgca

SEQ ID NO: 440 - DNA, 21 bp, artificial sequence
  synthetic polynucleotide
aacaagatcaccttctccgag

SEQ ID NO: 441 - DNA, 21 bp, artificial sequence
  synthetic polynucleotide
aactttgagaacatgagcaac

SEQ ID NO: 442 - DNA, 19 bp, artificial sequence
  synthetic polynucleotide
cgtggatttatggtctgtg

SEQ ID NO: 443 - RNA, 19 nt, artificial sequence
  synthetic polynucleotide
uggaugucuaucagcgcag

SEQ ID NO: 444 - RNA, 19 nt, artificial sequence
  synthetic polynucleotide
gcuacugccauccaaucga

SEQ ID NO: 445 - RNA, 19 nt, artificial sequence
  synthetic polynucleotide
ggaguacccugaugagauc

SEQ ID NO: 446 - RNA, 19 nt, artificial sequence
  synthetic polynucleotide
cugaggaguccaacaucac

SEQ ID NO: 447 - RNA, 19 nt, artificial sequence
  synthetic polynucleotide
ccaaggccagcacauagga

SEQ ID NO: 448 - DNA, 18 bp, artificial sequence
  synthetic polynucleotide
gtcgtgacttgcgacaag

SEQ ID NO: 449 - RNA, 19 nt, artificial sequence
  synthetic polynucleotide
gcuggcaguucauaggaau

SEQ ID NO: 450 - RNA, 19 nt, artificial sequence
  synthetic polynucleotide
cgugcuccaaagucuguua

SEQ ID NO: 451 - RNA, 19 nt, artificial sequence
  synthetic polynucleotide
ucaguauguugugcaagag

SEQ ID NO: 452 - RNA, 19 nt, artificial sequence
  synthetic polynucleotide
gaagauacucuggaauucc

SEQ ID NO: 453 - RNA, 19 nt, artificial sequence
  synthetic polynucleotide
cuggauuucacagagacug

SEQ ID NO: 454 - RNA, 19 nt, artificial sequence
  synthetic polynucleotide
gcagagaucauagagacug

SEQ ID NO: 455 - RNA, 19 nt, artificial sequence
  synthetic polynucleotide
gacuuuccucagaaugacg

SEQ ID NO: 456 - RNA, 19 nt, artificial sequence
  synthetic polynucleotide
cauuacuccaccuguauca

SEQ ID NO: 457 - RNA, 19 nt, artificial sequence
  synthetic polynucleotide
ggauuucaggaugaacacg

SEQ ID NO: 458 - RNA, 19 nt, artificial sequence
  synthetic polynucleotide
gcugcaggacuuccaccag

SEQ ID NO: 459 - RNA, 21 nt, artificial sequence
  synthetic polynucleotide
aauuggaacagcugcagcaga

SEQ ID NO: 460 - RNA, 21 nt, artificial sequence
  synthetic polynucleotide
aagcucugauuuaucaaaaga

SEQ ID NO: 461 - RNA, 19 nt, artificial sequence
  synthetic polynucleotide
cccacaccauuccauucua

SEQ ID NO: 462 - RNA, 23 nt, artificial sequence
  synthetic polynucleotide
aagaugaggaagaaaucgauguu

SEQ ID NO: 463 - RNA, 23 nt, artificial sequence
  synthetic polynucleotide
aaaaggucagagucuggaucacc

SEQ ID NO: 464 - RNA, 23 nt, artificial sequence
  synthetic polynucleotide
cacgucuccacacaucagcacaa

SEQ ID NO: 465 - RNA, 23 nt, artificial sequence
  synthetic polynucleotide
aaaugagauaaagguggcuaauu

SEQ ID NO: 466 - RNA, 19 nt, artificial sequence
  synthetic polynucleotide
ugguugcauuguccauggc

SEQ ID NO: 467 - DNA, 19 bp, artificial sequence
  synthetic polynucleotide
gatcgctgtgtgtctgtaa

SEQ ID NO: 468 - DNA, 19 bp, artificial sequence
  synthetic polynucleotide
gtccgtatgtaaatcagat

SEQ ID NO: 469 - DNA, 19 bp, artificial sequence
  synthetic polynucleotide
cataccatctctaccgacg

SEQ ID NO: 470 - DNA, 19 bp, artificial sequence
  synthetic polynucleotide
actgaagtctcggccagct

SEQ ID NO: 471 - DNA, 19 bp, artificial sequence
  synthetic polynucleotide
gaaactcgtcgcatcttcc

SEQ ID NO: 472 - RNA, 19 nt, artificial sequence
  synthetic polynucleotide
aucugcagaggccuccgca

SEQ ID NO: 473 - DNA, 19 bp, artificial sequence
  synthetic polynucleotide
gagcagaaccttcagaata

SEQ ID NO: 474 - DNA, 19 bp, artificial sequence
  synthetic polynucleotide
gagcagggcttcaccattg

SEQ ID NO: 475 - DNA, 19 bp, artificial sequence
  synthetic polynucleotide
ggaaggagaagaattcgta

SEQ ID NO: 476 - DNA, 19 bp, artificial sequence
  synthetic polynucleotide
gatatcatctttctctgaa

SEQ ID NO: 477 - DNA, 19 bp, artificial sequence
  synthetic polynucleotide
caactacggctttgccaat

SEQ ID NO: 478 - DNA, 19 bp, artificial sequence
  synthetic polynucleotide
gcaactcacgctccggaaa

SEQ ID NO: 479 - DNA, 19 bp, artificial sequence
  synthetic polynucleotide
gccctgagctggactactt

SEQ ID NO: 480 - DNA, 19 bp, artificial sequence
  synthetic polynucleotide
ggtatgcgacgggaaagta

SEQ ID NO: 481 - DNA, 21 bp, artificial sequence
  synthetic polynucleotide
aactcggaatccgaagttgga

SEQ ID NO: 482 - DNA, 21 bp, artificial sequence
  synthetic polynucleotide
aaagacctgagggaccgggag

SEQ ID NO: 483 - DNA, 19 bp, artificial sequence
  synthetic polynucleotide
gagaaaatgagctgtccgc

SEQ ID NO: 484 - DNA, 19 bp, artificial sequence
  synthetic polynucleotide
ctggcagaagtagcagaac

SEQ ID NO: 485 - RNA, 21 nt, artificial sequence
  synthetic polynucleotide
aaggcuuuggaacagaaacca

SEQ ID NO: 486 - RNA, 21 nt, artificial sequence
  synthetic polynucleotide
aagagucgggaccacaguuua

SEQ ID NO: 487 - RNA, 19 nt, artificial sequence
  synthetic polynucleotide
acagacuucggaguaccug

SEQ ID NO: 488 - RNA, 19 nt, artificial sequence
  synthetic polynucleotide
cggugcucaugcuuacaac

SEQ ID NO: 489 - RNA, 19 nt, artificial sequence
  synthetic polynucleotide
cgaguugcuagaccgcuuc

SEQ ID NO: 490 - RNA, 21 nt, artificial sequence
  synthetic polynucleotide
aaugccggugacacaacauga

SEQ ID NO: 491 - protein, 7 aa, artificial sequence
  synthetic polypeptide
RGDLXXX

SEQ ID NO: 492 - protein, 9 aa, artificial sequence
  synthetic polypeptide
CLSSRLDAC

SEQ ID NO: 493 - protein, 21 aa, artificial sequence
  synthetic polypeptide
WRCVLREGPAGGCAWFNRHRL

SEQ ID NO: 494 - protein, 13 aa, artificial sequence
  synthetic polypeptide
CGRECPRLCQSSC

SEQ ID NO: 495 - protein, 13 aa, artificial sequence
  synthetic polypeptide
CNGRCVSGCAGRC

SEQ ID NO: 496 - protein, 7 aa, artificial sequence
  synthetic polypeptide
CLPVASC

SEQ ID NO: 497 - protein, 8 aa, artificial sequence
  synthetic polypeptide
CLSGSLSC

SEQ ID NO: 498 - protein, 7 aa, artificial sequence
  synthetic polypeptide
GNKRTRG

SEQ ID NO: 499 - protein, 7 aa, artificial sequence
  synthetic polypeptide
CGAREMC

SEQ ID NO: 500 - protein, 7 aa, artificial sequence
  synthetic polypeptide
GGGVFWQ

SEQ ID NO: 501 - protein, 7 aa, artificial sequence
  synthetic polypeptide
HGRVRPH

SEQ ID NO: 502 - protein, 7 aa, artificial sequence
  synthetic polypeptide
VVLVTSS

SEQ ID NO: 503 - protein, 8 aa, artificial sequence
  synthetic polypeptide
CLHRGNSC

SEQ ID NO: 504 - protein, 12 aa, artificial sequence
  synthetic polypeptide
CRSWNKADNRSC

SEQ ID NO: 505 - protein, 20 aa, artificial sequence
  synthetic polypeptide
NAVPNLRGDLQVLAQKVART

SEQ ID NO: 506 - protein, 7 aa, artificial sequence
  synthetic polypeptide
MSTVGTG

SEQ ID NO: 507 - protein, 9 aa, artificial sequence
  synthetic polypeptide
CDCRGDCFC

SEQ ID NO: 508 - DNA, 60 bp, artificial sequence
  synthetic polynucleotide
aatgctgttcctaatttgagaggtgatttgcaagttttggctcaaaaagttgctagaact

SEQ ID NO: 509 - DNA, 60 bp, artificial sequence
  synthetic polynucleotide
ttacgacaaggattaaactctccactaaacgttcaaaaccgagtttttcaacgatcttga

SEQ ID NO: 510 - protein, 190 aa, artificial sequence
  synthetic polypeptide
MSTVGTGKLTRAQRRAAARKNKRNTHVVQPVIVEPIASGQGKAIKAWTGYSVSKWTASCAAAEAKVTSAITISLGNELSSERNKQLKVGRVLLWLGLLPSVSGTVKSCVTETQTTAAASFQVALAVADNSRDVVAAMYPEAFKGITLEQLTADLTIYLYSSAALTEGDVIVHLEVEHVRPTFDDSFTPVY

SEQ ID NO: 511 - DNA, 585 bp, artificial sequence
  synthetic polynucleotide
accggtatgagtacagtcggaacagggaagttaactcgtgcacaacgaagggctgcggcccgtaagaacaagcggaacactcacgtggtccaacctgttattgtagaacccatcgcttcaggccaaggcaaggctattaaagcatggacaggttacagcgtatcgaagtggaccgcctcttgtgcggctgccgaagctaaagtaacctcggctataactatctccctaggtaatgagctatcgtccgaaaggaacaagcagctcaaggtaggtagagttttattatggcttgggttgcttcccagtgttagtggcacagtgaaatcctgtgttacagagacgcagactactgctgctgcctcctttcaggtggcattagctgtggccgacaactcgagagatgttgtcgctgctatgtaccccgaggcgtttaagggtataacccttgaacaactcaccgcggatttaacgatctacttgtacagcagtgcggctctcactgagggcgacgtcatcgtgcatttggaggttgagcatgtcagacctacgtttgacgactctttcactccggtgtattaggtcgac

SEQ ID NO: 512 - DNA, 585 bp, artificial sequence
  synthetic polynucleotide
gtcgacctaatacaccggagtgaaagagtcgtcaaacgtaggtctgacatgctcaacctccaaatgcacgatgacgtcgccctcagtgagagccgcactgctgtacaagtagatcgttaaatccgcggtgagttgttcaagggttatacccttaaacgcctcggggtacatagcagcgacaacatctctcgagttgtcggccacagctaatgccacctgaaaggaggcagcagcagtagtctgcgtctctgtaacacaggatttcactgtgccactaacactgggaagcaacccaagccataataaaactctacctaccttgagctgcttgttcctttcggacgatagctcattacctagggagatagttatagccgaggttactttagcttcggcagccgcacaagaggcggtccacttcgatacgctgtaacctgtccatgctttaatagccttgccttggcctgaagcgatgggttctacaataacaggttggaccacgtgagtgttccgcttgttcttacgggccgcagcccttcgttgtgcacgagttaacttccctgttccgactgtactcataccggt

SEQ ID NO: 513 - protein, 29 aa, artificial sequence
  synthetic polypeptide
MSTVGTGKLTRAQRRLRARKNKRNTHVVQ

SEQ ID NO: 514 - DNA, 100 bp, artificial sequence
  synthetic polynucleotide
aggaattcaaaatgtctacagtcggaacagggaagttaactcgtgcacaacgaaggctgcgggcccgtaagaacaagcggaacactcacgtggtccaacc

SEQ ID NO: 515 - DNA, 100 bp, artificial sequence
  synthetic polynucleotide
ggttggaccacgtgagtgttccgcttgttcttacgggcccgcagccttcgttgtgcacgagttaacttccctgttccgactgtagacattttgaattcct

SEQ ID NO: 516 - protein, 29 aa, artificial sequence
  synthetic polypeptide
MSTVGTGVVQPVIVEPIASGQGKAIKAWT

SEQ ID NO: 517 - DNA, 100 bp, artificial sequence
  synthetic polynucleotide
aggaattcaaaaatgtctacagtcggaacaggggtggtccaacctgttattgtagaacccatcgcttcaggccaaggcaaggctattaaagcatggacag

SEQ ID NO: 518 - DNA, 100 bp, artificial sequence
  synthetic polynucleotide
ctgtccatgctttaatagccttgccttggcctgaagcgatgggttctacaataacaggttggaccacccctgttccgactgtagacatttttgaattcct

SEQ ID NO: 519 - protein, 33 aa, artificial sequence
  synthetic polypeptide
AITISLGNELSSERNKQLKVGRVLLWLGLLPSV

SEQ ID NO: 520 - DNA, 100 bp, artificial sequence
  synthetic polynucleotide
gctataactatctccctaggtaatgagctatcgtccgaaaggaacaagcagctcaaggtaggtagagttttattatggcttgggttgcttcccagtgtta

SEQ ID NO: 521 - DNA, 100 bp, artificial sequence
  synthetic polynucleotide
taacactgggaagcaacccaagccataataaaactctacctaccttgagctgcttgttcctttcggacgatagctcattacctagggagatagttatagc

SEQ ID NO: 522 - protein, 33 aa, artificial sequence
  synthetic polypeptide
AITISLGNAVPNLRGDLQVLAQKVARTLGNELS

SEQ ID NO: 523 - DNA, 100 bp, artificial sequence
  synthetic polynucleotide
gctataactatctccctagggaatgctgttcctaatttgagaggtgatttgcaagttttggctcaaaaagttgctagaactctaggtaatgagctatcgt

SEQ ID NO: 524 - DNA, 100 bp, artificial sequence
  synthetic polynucleotide
acgatagctcattacctagagttctagcaactttttgagccaaaacttgcaaatcacctctcaaattaggaacagcattccctagggagatagttatagc

SEQ ID NO: 525 - protein, 33 aa, artificial sequence
  synthetic polypeptide
AVADNSRDVVAAMYPEAFKGITLEQLTADLTIY

SEQ ID NO: 526 - DNA, 100 bp, artificial sequence
  synthetic polynucleotide
gctgtggccgacaactcgagagatgttgtcgctgctatgtaccccgaggcgtttaagggtataacccttgaacaactcaccgcggatttaacgatctact

SEQ ID NO: 527 - DNA, 100 bp, artificial sequence
  synthetic polynucleotide
agtagatcgttaaatccgcggtgagttgttcaagggttatacccttaaacgcctcggggtacatagcagcgacaacatctctcgagttgtcggccacagc

SEQ ID NO: 528 - protein, 33 aa, artificial sequence
  synthetic polypeptide
AVADNSNAVPNLRGDLQVLAQKVARTSRDVVAA

SEQ ID NO: 529 - DNA, 100 bp, artificial sequence
  synthetic polynucleotide
gctgtggccgacaactcgaatgctgttcctaatttgagaggtgatttgcaagttttggctcaaaaagttgctagaacttcgagagatgttgtcgctgcta

SEQ ID NO: 530 - DNA, 100 bp, artificial sequence
  synthetic polynucleotide
tagcagcgacaacatctctcgaagttctagcaactttttgagccaaaacttgcaaatcacctctcaaattaggaacagcattcgagttgtcggccacagc